(19) United States
(12) Patent Application Publication
(10) Pub. No.: US 2025/0289857 A1
(43) Pub. Date: Sep. 18, 2025

(54) ALTERNATIVE SPLICING ENHANCER MOLECULES

(71) Applicant: The Regents of the University of California, Oakland, CA (US)

(72) Inventors: Eugene Yeo, La Jolla, CA (US); Jonathan Schmok, Sunnyvale, CA (US)

(21) Appl. No.: 19/076,526

(22) Filed: Mar. 11, 2025

Related U.S. Application Data
(60) Provisional application No. 63/564,396, filed on Mar. 12, 2024.

(51) Int. Cl. C07K 14/47; C12N 9/22

(52) U.S. Cl. CPC C07K 14/47; C12N 9/22; C07K 2319/00

(57) ABSTRACT

Provided herein is a molecule for use in directing splicing event. More specifically, provided herein is a molecule for use in targeted exon inclusion during mRNA splicing, along with compositions and methods of using the same.

### CROSS-REFERENCE TO RELATED APPLICATIONS

This application claims the benefit of priority to U.S. Provisional Patent Application No. 63/564,396, filed on Mar. 12, 2024, the contents of which are incorporated herein by reference in their entirety.

### STATEMENT OF GOVERNMENT SUPPORT

This invention was made with government support under Grant No. HG004659 awarded by the National Institutes of Health. The government has certain rights in the invention.

### BACKGROUND OF THE DISCLOSURE

Alternative splicing is a co- and post-transcriptional processing step in which certain regions of transcribed pre-mRNA are included or skipped from the final mRNA product depending on the regulatory environment at the time of processing. Alternative splicing is a major source of expanded protein diversity over the raw number of genes in the genome, with virtually all human protein-coding genes expressing multiple alternatively spliced isoforms. Mis-regulation of this process has been implicated in many diseases.

Targeted modulation of alternative splicing presents an opportunity for both therapeutics, such as to correct mis-splicing of specific RNA targets, and scientific inquiry, such as to functionally probe individual splicing events. Approaches have been taken to induce the skipping of alternative exons. Targeted inclusion of exons, however, is less common as the blunt approaches of inhibiting splicing mechanisms by targeted destruction or steric inhibition of splicing signals at the target does not function effectively for targeted inclusion methods.

### SUMMARY OF THE DISCLOSURE

The following description of the background of the present technology is provided simply as an aid in understanding the present technology and is not admitted to describe or constitute prior art to the present technology.

The present disclosure provides a molecule and methods of using the molecule to direct targeted exon inclusions during mRNA splicing. In some illustrations, the molecule allows for the production of misspliced mRNA molecules to facilitate the production of wild-type protein to treat a disease. It is to be understood that the disclosed illustrations are merely exemplary, and accordingly, the invention may be embodied in various and alternative forms.

In a first illustration, provided herein is a targeted exon inclusion molecule comprising an optimized effect domain of an RNA-binding protein and an RNA-targeting moiety.

In a second illustration, provided herein is a method of generating targeted exon inclusion molecules, the method comprising, or consisting essentially of, or consisting of: (a) generating a plurality of first fusion proteins, wherein each first fusion protein comprises an RNA-binding protein or fragment thereof and a reporter binding domain; (b) transfecting each of the first fusion proteins with a reporter construct, wherein the reporter construct comprises an mRNA sequence encoding a first reporter gene, a target exon comprising an in-frame stop codon, a reporter binding domain recognition sequence, and a second reporter gene, wherein the target exon is between the first reporter gene and the second reporter gene, and wherein the reporter biding domain recognition sequence is either 30 nucleotides upstream or downstream of the target exon; (c) measuring the relative ratios of expression of the second reporter gene to expression of the first reporter gene for each transfection; (d) selecting the first fusion proteins that effectively direct targeted exon inclusion during splicing of the reporter construct, wherein effective direction of targeted exon inclusion is determined by a lower ratio of expression of the second reporter gene to expression of the first reporter gene; and (e) subcloning each RNA-binding protein or fragment thereof of the selected first fusion proteins with a tiling approach across the whole length of the RNA-binding protein or fragment thereof, or fusing each RNA-binding protein or fragment thereof to an RNA-targeting moiety to generate targeted exon inclusion molecules.

The specific structural and functional details disclosed herein are not to be interpreted as limiting but merely as a basis for the claims and as a representative basis for teaching one skilled in the art to variously employ the illustrations described herein. Further objectives and advantages of the technology will be clear from the description that follows.

### BRIEF DESCRIPTION OF THE DRAWINGS

FIGS. 1A-1B: FIGS. 1A and 1B show the engineered dual-luciferase reporters used in the high-throughput screens of RNA-binding proteins. FIG. 1A shows the schematic of the luc-MAPT-30D reporter used to identify RNA-binding proteins that induce exon inclusion when recruited 30 base pairs downstream of the alternatively spliced exon of interest along with the processed mRNA constructs generated when the exon is skipped or when the exon is included. FIG. 1B shows the schematic of the luc-MAPT-30U used to identify RNA-binding proteins that induce exon skipping when recruited 30 base pairs upstream of the alternatively spliced exon of interest along with the processed mRNA constructs generated when the exon is skipped or when the exon is included. See also FIG. 6A.

FIG. 2: FIG. 2 depicts the experimental schematic and workflow for the high-throughput screen of RNA-binding proteins. Each of the reporters is co-transfected using Lipofectamine 3000 with one RNA-binding protein (RBP)/MS2-coat protein (MCP) (RBP/MCP) fusion protein in each well of a 96-well plate. Each combination is tested in triplicate, with a triplicate of negative controls (FLAG epitope tag fused to MCP) and positive control (RBFOX1 for lucMAPT-30D and SRSF5 for lucMAPT-30U) on each experimental plate. Change in percent spliced in (PSI/ψ) is estimated per the provided formula. Each candidate that passed the initial round with a p-value of 0.05 or less by the one-tailed t-test was again tested in triplicate for a validation round.

FIGS. 3A-3B: FIG. 3 depicts the sample 96-well plates used in the screen for the upstream inclusion reporter (lucMAPT-30U) (FIG. 3A) and the downstream inclusion reporter (lucMAPT-30D) (FIG. 3B). Each 96-well plate contains a negative control (NEG), positive control (SRSF5 for lucMAPT-30U, RBFOX1 for lucMAPT-30D), and a series of experimental RNA-binding protein/MS2 coat protein fusions in triplicate. PSI is inferred from the previously described formula, and changes are visualized here (mean +/− standard deviation).

FIGS. 4A-4B: FIG. 4 depicts the inferred PSI (mean +/− standard deviation) from the validation round for all RNA-binding proteins that showed significant upregulation of exon inclusion using the upstream inclusion reporter (lucMAPT-30U) (FIG. 4A) and the downstream inclusion reporter (lucMAPT-30D) (FIG. 4B) in the validation round of the high-throughout screen of RNA-binding proteins. Candidates are sorted by strength. Validated hits that contain previous gene ontology (GO) annotations as splicing factors are in blue, and hits that do not have previous GO annotations as splicing factors and are potentially novel are in yellow.

FIG. 5: FIG. 5 shows the experimental schematic and workflow for engineering optimized effector domains from candidate RNA-binding proteins. The strongest candidate RNA-binding proteins are identified from the screen and are used in developing optimized effector domains for potency and delivery size. Predicted domains from these candidates are identified and fused with the MS2-coat protein (MCP). These are, in turn, assayed with the same reporter system. Domains that retain potent activity are further optimized for delivery size through a tiling approach. These engineered effectors are coupled with an RNA-targeting protein to create artificial splicing factors and induce the inclusion of endogenous exons.

FIGS. 6A-6E: Development of tethered function assays for detecting direct induction of exon inclusion. (FIG. 6A) Schematic of luciferase reporters used in the assays and resulting isoforms following cellular mRNA processing. (FIG. 6B) Analysis workflow for calculating percent-spliced-in from luminescence measurements. (FIG. 6C) Splicing gels of lucMAPT-30D splicing in response to co-transfection with MCP-fused positive and negative controls. Bands are generated by agarose gel electrophoresis of RT-generated cDNA amplified by minigene specific primers (shown in panel a) that amplify skipping and inclusion isoforms. (FIG. 6D) Bar graph of lucMAPT-30D reporter readout as calculated from the workflow in panel b with the same conditions as panel c (mean±s.d., n=3 replicate transfections). (FIG. 6E) Experimental workflow of tethering assays. The effects of recruiting 718 MCP-fused RBPs are tested in both reporter contexts. P-value is calculated by independent two-sample one-tailed 1-test, comparing the co-transfection of the reporter and candidates to co-transfection of the reporter and FLAG NC performed concurrently. The displayed n refers to biological replicates of candidate transfections. For FLAG NC transfections, n=3 biological replicates for the reporter experiments and n=6 for the splicing gel experiment. Venn diagram of final hits following all rounds of screening and verification.

FIGS. 7A-7F: Tethering assays identify RNA-binding proteins that induce exon inclusion. (FIG. 7A) Bar graph displaying gene ontology analysis of all hits emerging from the screen. The top four mostly significantly enriched biological processes are displayed. Background for analysis is the complete list of genes used in the screens. Unadjusted p-value is calculated by Metascape⁵⁶ based on the accumulative hypergeometric distribution. (FIG. 7B) Bar graph displaying manual annotation of protein families displaying reporter preferences in the screen and reporter preferences of unexpected hits. (FIG. 7C, FIG. 7D, FIG. 7E) Bar graphs displaying reporter readout following screen and validation for RBP-MCP fusions of all final hits passing the lucMAPT-30U screen only (FIG. 7C), the lucMAPT-30D screen only (FIG. 7D), and both screens (FIG. 7E) (mean±s.d., n=3 replicate transfections). (FIG. 7F) Scatter plot comparing activation of both reporters of all hits emerging from the screen. Hits that only passed for a single reporter are placed along the axis for the other reporter. Hits that passed the lucMAPT-30U screen only are displayed as orange markers, hits that passed the lucMAPT-30D screen only are displayed as blue markers, and hits that passed both screens are displayed as green markers.

FIGS. 8A-8J: Integrated analysis of eCLIP and KD RNA-Seq reveals splicing events modulated by unexpected hits from the tethering assay. (FIG. 8A) Stacked bar graph displaying distribution of RNA binding positions of unexpected hits identified by eCLIP and Skipper analysis (n=2 for IP and sized-matched input). (FIG. 8B) The most significantly enriched RNA binding motifs as identified by HOMER⁵⁷ analysis of eCLIP signal of unexpected hits along with corresponding p-value, as calculated by the HOMER algorithm using default settings. (FIG. 8C) Stacked bar graph of differentially spliced events following shRNA-mediated knockdown of unexpected hits as identified by rMATS analysis of RNA-seq data. Differentially spliced events are called as those with an inclusion level difference >0.05 and a multiple hypothesis adjusted p-value of <0.05 as calculated by likelihood-ratio test. (FIG. 8D) Stacked bar graph of Skipped Exon events following shRNA-mediated knockdown of unexpected hits as identified by rMATS analysis of RNA-seq data. Differentially spliced events are called as those with an inclusion level difference >0.05 and a multiple hypothesis adjusted p-value of <0.05 as calculated by likelihood-ratio test. Events called as ‘Skipping after knockdown’ are differentially spliced events with IncLevelKD−IncLevelNT <0. Events called as ‘Inclusion after knockdown’ are differentially spliced events with IncLevelKD−IncLevelNT >0. (FIG. 8E) Bar graph showing the fraction of genes containing significantly enriched windows following eCLIP of unexpected hits, binned into genes containing corresponding KD-sensitive skipped exons events and those without. P-value is calculated by one-tailed Fisher Exact Test. (FIG. 8F) Venn diagrams displaying the number of genes containing differentially spliced skipped exon events following unexpected hit KD and the number of genes containing significantly enriched windows following the corresponding eCLIP. (FIGS. 8G-8J) Scatter plots examining genes containing unexpected hit KD-sensitive skipped exon events and corresponding significantly enriched binding windows. Binding positions were stratified by feature relative to the skipped exon. When multiple binding windows were identified on the same feature relative to an exon, the median binding window is displayed. (FIG. 8G) STAU2. (FIG. 8H) SCAF8. (FIG. 8I) RTCA. (FIG. 8J) TRNAU1AP.

FIGS. 9A-9D: AP-MS identifies splicing-associated proteins following pull-down of candidate proteins. (FIG. 9A) Hierarchically clustered heatmap displaying Z-scores from AP-MS of annotated splicing-associated preys that were detected with a Z-score>2 as calculated by Spectronaut by mass spectrometry following affinity purification of any of the baits. Preys are displayed on the y-axis and baits are displayed on the x-axis. (FIG. 9B) Bar graphs displaying gene ontology analysis of preys detected by mass spectrometry following affinity purification of the unexpected hits from the tethering assay. Significantly enriched preys for GO analysis are defined as having q value <0.05 and log 2 ratio IP/FLAG>1 (multiple-hypothesis corrected, determined by Spectronaut algorithm). Splicing-associated GO terms are highlighted in red. Gaps in the y-axis are used to visualize the most highly enriched splicing-associated GO term when one wasn't present in the top four. Unadjusted p-value of enrichment is calculated by Metascape⁵⁶ based on the accumulative hypergeometric distribution. (FIGS. 9C-9D) Stacked bar graphs displaying the overall count of preys significantly detected in follow-up experiments over IgG controls (fold change >0.5, unadjusted p-value <0.00000001 as determined by Spectromine algorithm), separated by interaction type (see methods). The count of preys that are annotated as RBPs (panel FIG. 9C) and contain RNA splicing GO terms (panel FIG. 9D) are displayed.

FIGS. 10A-10K: TRNAU1AP participates in splicing co-regulatory networks and activates exon inclusion through a C-terminal effector domain. (FIG. 10A) Bar graph showing relative expression level of the top 10 differentially expressed splicing-associated genes as sorted by DeSeq2-determined adjusted p-value following TRNAU1AP KD (mean±s.d., n=3 replicate transductions). (FIG. 10B) Bar graph showing relative exon inclusion level of the top 10 differentially spliced skipped exon events in splicing-associated genes as sorted by rMATS-determined adjusted p-value following TRNAU1AP KD (mean±s.d., n=3 replicate transductions). (FIG. 10C) IGV browser tracks showing coverage of TRNAU1AP eCLIP signal relative to sized-matched input and TRNAU1AP KD RNA-Seq signal relative to non-targeting shRNA at a poison exon in PRPF39, exon 2 of HNRNPA2B1, and exon 5 of MBZL.1 (FIG. 10D) Representative western blot showing increased PRPF39 expression in HEK293T cells following TRNAU1AP knockdown. GAPDH is the loading control. (FIG. 10E) Bar graph showing fold change of PRPF39 expression as quantified by western blot following TRNAU1AP knockdown (mean±s.d., n=3 replicate transfections). p=0.0024 by two-tailed independent two-sample t-test. (FIG. 10F) Bar plot displaying percentage of exons containing PRPF39 reproducible enriched eCLIP windows in flanking introns from ENCODE HepG2 data, separated by exon sensitivity to TRNAU1AP KD in HEK293T cells. p values are calculated using the two-sided chi-squared test. p=0.0011 for PRPF39 binding to exons skipped after TRNAU1AP knockdown and 0.0088 for exons included after TRNAU1AP knockdown. (FIG. 10G) Domain structure of TRNAU1AP with truncations used for effector domain identification. (FIG. 10H) Bar graphs displaying reporter readout from both lucMAPT-30U and lucMAPT-30U co-transfected with MCP-fused truncations (mean±s.d., n=3 replicate transfections). P-value is calculated by one-tailed independent two-sample t-test. ns=not significant (p>0.05). (FIG. 10I) Schematic of truncation-dCas13d fusions used as for MS2-free tests. Schematic of MS2-free lucMAPT reporter used and associated guide RNAs. (FIG. 10J, FIG. 10K) Reporter readouts from co-transfection of the MS2-free lucMAPT reporter, either full-length TRNAU1AP-dCas13d fusion or truncated TRNAU1AP-5-dCas13d fusion, and each guide RNA annotated in panel i). (FIG. 10J) Bar graph showing PSI calculated from luminescence (mean±s.d., n=3 replicate transfections). P-value is calculated by one-tailed independent two-sample t-test. ns=not significant (p>0.05). (FIG. 10K) Splicing gels displaying lucMAPT alternative splicing.

FIGS. 11A-11H: Truncation of the top RBP hits identify splice enhancing domains that can be repurposed for artificial splicing factors. (FIGS. 11A-11C) Domain structures of top hits used for truncation experiments, D-NTD and D-CTD represent N-terminal and C-terminal domains, respectively, containing Mobidblt-consensus disorder prediction. All tested truncations are shown. Hits are separated into their position-dependence from the initial screen: position independent hits (FIG. 11A), hits that primarily activated the lucMAPT-30D reporter (FIG. 11B), and hits that primarily activated the lucMAPT-30U reporter (FIG. 11C). (FIGS. 11D-11F) Bar graphs displaying reporter readout from both lucMAPT-30U and lucMAPT-30U of the full-length proteins next to their associated truncations (mean±s.d., n=3 replicate transfections). Graphs are separated by position-dependence of full-length protein from the initial screen as in panels a-c. (FIG. 11G) (left, top) Schematic of truncation-dCas13d fusion used as artificial splicing factors. (left, bottom) Schematic of MS2-free lucMAPT reporter used for reporter-based assessment of artificial splicing factors. (right) Bar graphs displaying reporter output from MS2-free lucMAPT reporter following co-transfection of reporter with truncation-dCas13d fusion, and gRNA containing plasmid (mean±s.d., n=3 replicate transfections). (FIG. 11H) (left, top) Schematic of truncation-dCas13d fusion used as artificial splicing factor. (left, bottom) Schematic of HNRNPD Exon 7 used for endogenous splicing modulation, with the position of the two sets of three gRNAs that are co-transfected with the artificial splicing factors as gRNA-arrays. (middle) Agarose gel showing splicing of HNRNPD Exon 7 of a sample replicate for both artificial splicing factors in co-transfection with both gRNA arrays and a non-targeting gRNA (NT). (right) Bar graphs displaying quantification of inclusion/exclusion ratio normalized to the non-targeting gRNA (NT) from gels in Extended Data FIG. 6c-d (mean±s.d., n=3 replicate transfections).

FIGS. 12A-12G: Reporter construction strategy, tethering validation, reporter layout, splicing gels. (FIG. 12A) Schematic of strategy used for assembling luciferase based minigene splicing reporters. (FIG. 12B) Bar graph of lucMAPT-30D reporter readout following co-transfection with FLAG NC, RBFOX1-MCP fusion (RBFOX1), and RBFOX1 lacking an MCP fusion (RBFOX1 NoMS2) (mean±s.d., n=3 replicate transfections). (FIG. 12C) Western blots for validation of UPF1 shRNA constructs qualitatively showing decreased UPF1 protein levels for each of four UPF1 shRNA constructs tested in HEK293T cells. (FIG. 12D) qPCR for validation of SMG7 shRNA constructs showing decreased SMG7 expression levels as quantified using the delta-delta Ct method in RNA extracted from MDAMB231 and MCF10A cells stably expressing the constructs (n=2 biological replicates (1 replicate/line), n=2 technical replicates). (FIG. 12E) Bar graph of reporter readouts in HEK293T cells stably expressing a non-targeting shRNA (NT), a UPF1-targeting shRNA (sh302), and two SMG7-targeting shRNAs (sh65 and sh88), co-transfected with reporter plasmids and FLAG NC (mean±s.d., n=6 replicate transfections). P-value is calculated by two-tailed independent two-sample t-test. (FIG. 12F) Layout of 96-well transfections used throughout the screens. (FIG. 12G) Agarose gels of RNA-level validation of hits from the splicing screen. All hits were tested for lucMAPT-30D (top) and lucMAPT-30U (bottom). Numbers along the top correspond to lane number in Supplementary Table 6-7 see Schmok J C, et al. (2024), incorporated herein by reference. n=2 replicate transfections.

FIGS. 13A-13F: Survey of screen hits with complementary reporters. (FIG. 13A) Schematic of luciferase reporters for tethering 100 base pairs away from the splice site. (FIG. 13B) Clustered bar graph of upstream tethering only hits from the screen comparing results from the original screen (lucMAPT-30U) to results from co-transfection of the RBP-MCP fusions and lucMAPT-100U (mean±s.d., n=3 replicate transfections). (FIG. 13C) Clustered bar graph of downstream tethering only hits from the screen comparing results from the original screen (lucMAPT-30D) to results from co-transfection of the RBP-MCP fusions and lucMAPT-100D (mean±s.d., n=3 replicate transfections). (FIG. 13D) Clustered bar graph of hits that activated both reporters from the screen comparing results from the original screens (lucMAPT-30D, lucMAPT-30U) to results from co-transfection of the RBP-MCP fusions and the long-distance reporters (lucMAPT-100D and lucMAPT-100U) (mean±s.d., n=3 replicate transfections). Results where hits displayed a mean ψ from luminescence <0 are omitted for clarity. (FIG. 13E) Schematic of lucMBNL1 reporters used as orthogonal exon inclusion reporters. (FIG. 13F) Bar graphs of reporter readout from co-transfection of all hits from the original screens with lucMBNL1-30D and lucMBNL1-30U (mean±s.d., n=3 replicate transfections).

FIGS. 14A-14D: Exon skipping screen. (FIG. 14A) Schematic of luciferase reporters for skipping readout. (FIG. 14B) lucMAP3K7-100U splicing in response to co-transfection with MCP-fused positive and negative controls. (left) Bar graph of lucMAP3K7 reporter readout (mean±s.d., n=3 replicate transfections). (right) Agarose gel electrophoresis of RT-generated cDNA amplified by minigene specific primers (shown in panel a) that amplify skipping and inclusion isoforms. (FIG. 14C) Bar graph of lucMAP3K7-30D reporter readout when co-transfected with RBP-MCP fusions from the library. (mean±s.d., n=3 replicate transfections). (FIG. 14D) Bar graph of lucMAP3K7-100U reporter readout when co-transfected with RBP-MCP fusions from the library (mean±s.d., n=3 replicate transfections).

FIGS. 15A-15D: Quality control of eCLIP and shRNA knockdown followed by RNA-seq. (FIG. 15A) Western blots of cold gels from eCLIP protocol for TRNAU1AP, SCAF8, STAU2 and RTCA. Size-matched input and immunoprecipitation conditions are compared. n=2 independent samples, with size-matched input and IP conditions extracted from both. (FIG. 15B) Mosaic plots from Skipper showing concordance between eCLIP replicates. Odds ratios and significance from Fisher's exact test. (FIG. 15C) TPM of unexpected hits following shRNA knockdown as measured from aligned RNA-seq data. (mean±s.d., n=3 replicate knockdowns). (FIG. 15D) IGV browser tracks showing coverage of RBP eCLIP signal relative to sized-matched input and the RBP KD RNA-Seq signal relative to non-targeting shRNA. From left to right: comparison of TRNAU1AP eCLIP and KD RNA-Seq signal near MBZL Exon 5, comparison of RTCA eCLIP and KD RNA-Seq signal near LRIF Exon 2, comparison of SCAF8 eCLIP and KD RNA-Seq signal near METTL26 Exon 2, comparison of STAU2 eCLIP and KD RNA-Seq signal near SENP3 Exon 6.

FIGS. 16A-16H: Quality control of AP-MS. (FIGS. 16A-16H) Scatter plots showing concordance between AP-MS replicates. Each point represents a detected protein and its z-score in two replicates per plot. Red points represent the detection of the bait protein among the preys. Multiple red points indicate multiple major isoforms detected with average Z-score>1. (FIG. 16A) FLAG NC. (FIG. 16B) TRANAU1AP. (FIG. 16C) RTCA. (FIG. 16D) SCAF8. (FIG. 16E) STAU2. (FIG. 16F) CLK2. (FIG. 16G) PRKRA. (FIG. 16H) GPATCH2.

FIGS. 17A-17D: Full western blots and splicing gels for TRNAU1AP follow-up experiments and modulation of endogenous HNRNPD Exon 7. (FIG. 17A) Western blot replicates used for quantification showing increased PRPF39 expression in HEK293T cells following TRNAU1AP knockdown. GAPDH is the loading control. n=3 independent transductions. (FIG. 17B) Additional replicate displaying lucMAPT alternative splicing from co-transfection of the MS2-free lucMAPT reporter, either full-length TRNAU1AP-dCas13d fusion or truncated TRNAU1AP-5-dCas13d fusion, and each reporter targeting guide RNA annotated in FIG. 10I. n=2 independent transfections (FIGS. 17C-17D) Agarose gels of amplified cDNA collected from HEK293T cells co-transfected with artificial splicing factors (RBFOX1-dCasRx-C, SRSF8-2) and gRNA arrays (NT=non-targeting gRNA, DN=downstream 3-gRNA array, UP=upstream 3-gRNA array). n=3 independent transfections.

### DETAILED DESCRIPTION OF THE DISCLOSURE

One proposed method of driving targeted inclusion of exons during splicing is inspired by splicing factors, a category of RNA-binding proteins that influence alternative splicing outcomes. These splicing factors are trans-acting and act to enhance or silence exon inclusion by binding near or on the target exon and promoting or repressing the activity of splicing machinery.

A new scalable approach to identify known and novel proteins that enhance inclusion of alternatively spliced exons when placed in proximity to the target exon has been developed and is disclosed herein. The approach is informed by a large-scale assay of naturally occurring or natural expressed human RNA-binding proteins to determine the strongest splicing factors that, when tethered close to a target exon, causes inclusion of the target exon.

The identified naturally occurring candidates can be further utilized to generate optimized synthetic, targeted systems for tunable strength and minimal size. The initially identified candidates are also disclosed herein, but additional candidate proteins can be identified by the approach provided. These candidates can be further optimized to generate alternative splicing enhancers that include smaller portions of the RNA-binding proteins to facilitate easier delivery of the enhancer to cells while maintaining potent and robust RNA binding activity.

This innovation can be used for many applications depending on the specifics of the target exons, including, but not limited to, modulation of gene expression and correction of aberrant splicing.

In a first illustration, provided herein is a targeted exon inclusion molecule comprising, or consisting essentially of, or alternatively consisting of an optimized effect domain of an RNA-binding protein and an RNA-targeting moiety. In some aspects of the first illustration, the RNA-binding protein is selected from the group consisting of: SRSF8, RNPS1, SRSF10, SRSF4, SRSF5, SREK1, LUC7L2, SRSF6, SNIP1, U2AF2, GTF2F1, RBM25, STAU2, MAZ, CLK3, THRAP3, FIL1L1, MBNL1, SNRNP70, DDX23, XPO1, UBAP2L, SRSF12, RMBX2, SRSF11, PUG60, SNW1, METTL16, SF1, STAU1, CNOT3, EIF4B, SNRPN, SNRPB, SNRPA, RBM5, SNRNP40, RSRC1, TIAL1, FUBP1, SNURF, SRSF7, TRNAU1AP, CCNL1, SNRPE, RBFOX1, RBFOX2, KIAA1967, SNRPG, RTCA, CLK2, PRKRA, SCAF8, SF3A2, PCBP1, SF3B4, RBM38, RY1, and CELF3. In some aspects of the first illustration, the RNA-targeting moiety is selected from the group of Cas13, Cas13 proteins with modifications to reduce immunogenicity, Pumilio (PUF) RNA binding proteins, antisense oligonucleotides (ASOs), or small molecule compounds. In some aspects of the first illustration, provided herein are polynucleotides encoding the targeted exon inclusion molecule. In some aspects of the first illustration, provided herein are vectors or isolated host cells comprising the polynucleotide encoding the targeted exon inclusion molecule. In some aspects of the first illustration, provided herein are compositions comprising, or consisting essentially of, or consisting of, the targeted exon inclusion molecule and a carrier. In some aspects of the first illustration, provided herein are compositions comprising, or consisting essentially of, or consisting of, the polynucleotide encoding the targeted exon inclusion molecule and a carrier.

In some aspects of the first illustration, provided herein is a method of targeted exon inclusion, the method comprising, or consisting essentially of, or consisting of, contacting a cell comprising a messenger RNA (mRNA) target with the targeted exon inclusion molecule, under conditions to allow the targeted exon inclusion molecule to bind to the mRNA target and facilitate inclusion of a target exon during splicing of the mRNA. As used herein, the term “contacting” intends placing two or more agents in close proximity such that a molecular reaction or biological process can take place. In some aspects, one or more agents, compounds or molecules can be added to a reaction vesicle or cell culture medium. Alternatively, contacting in vivo can be administration of a agent, compound or molecule locally or systemically to a subject such that the agent, compound or molecule is delivered in vivo to the site of action or injury. Thus, in some further aspects of this first illustration, the contacting of the cell occurs in vitro or in vivo. In some further aspects of the first illustration, the cell is a mammalian cell. In some further aspects of this first illustration, the mammalian cell is contract in vitro and is a muscle cell. In some further aspects of this first illustration, the muscle cell is from a subject with muscular dystrophy.

In some further aspects of this first illustration, the mRNA bound by the targeted exon inclusion molecule is dystrophin or a functional fragment thereof. In some further aspects of this first illustration, the method further comprises measuring the expression of the polynucleotide comprising, or consisting essentially of, or consisting of, the target exon. In some further aspects of this first illustration, the method further comprises measuring the levels of dystrophin protein produced from mRNA that include the target exon. In some further aspects of this first illustration, the target sequence that is recognized by the RNA-targeting moiety of the targeted exon inclusion molecule is at most 30 nucleotides upstream or downstream of the target exon.

In a second illustration, provided herein is a method of generating targeted exon inclusion molecules, the method comprising, or consisting essentially of, or consisting of: (a) generating a plurality of first fusion proteins, wherein each first fusion protein comprises an RNA-binding protein or fragment thereof and a reporter binding domain; (b) transfecting each of the first fusion proteins with a reporter construct, wherein the reporter construct comprises an mRNA sequence encoding a first reporter gene, a target exon comprising an in-frame stop codon, a reporter binding domain recognition sequence, and a second reporter gene, wherein the target exon is between the first reporter gene and the second reporter gene, and wherein the reporter biding domain recognition sequence is either 30 nucleotides upstream or downstream of the target exon; (c) measuring the relative ratios of expression of the second reporter gene to expression of the first reporter gene for each transfection; (d) selecting the first fusion proteins that effectively direct targeted exon inclusion during splicing of the reporter construct, wherein effective direction of targeted exon inclusion is determined by a lower ratio of expression of the second reporter gene to expression of the first reporter gene; and (e) subcloning each RNA-binding protein or fragment thereof of the selected first fusion proteins with a tiling approach across the whole length of the RNA-binding protein or fragment thereof, or fusing each RNA-binding protein or fragment thereof to an RNA-targeting moiety to generate targeted exon inclusion molecules.

In some aspects of this second illustration, wherein upon subcloning each RNA-binding protein or fragment thereof, the method further comprises, or consists essentially of, or consists of: (f) generating a plurality of second fusion proteins, wherein each second fusion protein comprises a subcloned portion of the RNA-binding proteins or fragments thereof and the reporter binding domain; (g) transfecting each of the second fusion proteins with the reporter construct; (h) measuring the relative ratios of expression of the second reporter gene to the first reporter gene for each transfusion; (i) selecting the second fusion proteins that effectively direct targeted exon inclusion during splicing of the reporter construct; and (j) fusing each subcloned portion of the RNA-binding proteins or fragments thereof to an RNA-targeting moiety to generate additional targeted exon inclusion molecules.

### Definitions

Unless defined otherwise, all technical and scientific terms used herein generally have the same meaning as commonly understood by one of ordinary skill in the art to which this technology belongs. As used in this specification and the appended claims, the singular forms “a,” “an,” and “the” include plural referents unless the context clearly dictates otherwise. For example, reference to a “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “comprising” is intended to mean that the compositions and methods include the recited elements, but do not exclude others. As used herein, the transitional phrase “consisting essentially of” (and grammatical variants) is to be interpreted as encompassing the recited materials or steps “and those that do not materially affect the basic and novel characteristic(s)” of the recited embodiment. Thus, the term “consisting essentially of” as used herein should not be interpreted as equivalent to “comprising.” For example, the gene editing systems described herein may consist essentially of the recited materials and additional materials that do not affect the ability of the at least one gRNA to hybridize to a nucleotide sequence complementary to a target sequence or to associate with the E gene or N gene. “Consisting of” shall mean excluding more than trace elements of other ingredients and substantial method steps for administering the compositions disclosed herein. Aspects defined by each of these transition terms are within the scope of the present disclosure.

As used herein, “about” when used with a numerical value means the numerical value stated as well as plus or minus 10% of the numerical value (except where such number would be less than 0% or exceed 100% of a possible value). For example, “about 10” should be understood as both “10” and “9-11.

As used herein, the “administration” of or “administering” an agent to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

As used herein, the term “CRISPR” refers to Clustered Regularly Interspaced Short Palindromic Repeats (CRISPR). CRISPR may also refer to a gene editing system or technique relying on CRISPR-based, sequence-specific genetic or epigenetic manipulation. Epigenetic manipulation includes modifications to nucleotides or higher order chromatin structure that can alter expression patterns of genes in the absence of changes to the underlying DNA sequence. Epigenetic modifications can occur on multiple levels, such as 5-methyl-cytosine (5-meC) DNA methylation, post-translational modifications of histones bound by protein domains that serve as epigenetic writers, readers and erasers, and non-coding RNAs that assist in the recruitment of chromatin modifying proteins to DNA. A CRISPR-based gene editing system can also be programmed to cleave a target polynucleotide using a CRISPR endonuclease and a guide RNA. A CRISPR system can be used to cause double stranded or single stranded breaks in a target polynucleotide. A CRISPR system can also be used to recruit proteins or label a target polynucleotide. In some aspects, CRISPR-mediated gene editing utilizes the pathways of nonhomologous end-joining (NHEJ) or homologous recombination to perform the edits. These applications of CRISPR technology are known and widely practiced in the art. See, e.g., U.S. Pat. No. 8,697,359; Int'l. Publ. Nos. WO 2017/091630 A1, WO 2017/180915 A2, WO 2018/035503 A1, and WO 2018/170015 A1; Hsu et al. (2014) Cell 156 (6): 1262-78; and Urbano et al. (2019) Cancers 11 (10): E1515.

The term “Cas9” refers to a CRISPR-associated, RNA-guided endonuclease such as Streptococcus pyogenes Cas9 (spCas9) and orthologs and biological equivalents thereof. Biological equivalents of Cas9 include but are not limited to C2c1 from Alicyclobacillus acideterrestris and Cpf1 (which performs cutting functions analogous to Cas9) from various bacterial species including Acidaminococcus spp. and Francisella novicida U112. Cas9 may refer to an endonuclease that causes double stranded breaks in DNA, a nickase variant such as a RuvC or HNH mutant that causes a single stranded break in DNA, as well as other variations such as deadCas-9 or dCas9, which lack endonuclease activity. Cas9 may also refer to “split-Cas9” in which CAs9 is split into two halves—C-Cas9 and N-Cas9—and fused with a two intein moieties. See, e.g., U.S. Pat. No. 9,074,199 B1; Zetsche et al. (2015) Nat Biotechnol. 33 (2): 139-42; Wright et al. (2015) PNAS 112 (10) 2984-89. An additional example includes CRISPR associated endonucleoase referred to by this name (UniProtKB G3ECR1 (CAS9_STRTR)) as well as deadCas-9 or dCas9, which lacks endonuclease activity.

The term “Cas13” refers to a family of type of RNA targeting enzymes. The diverse Cas13 family contains at least four known subtypes, including Cas13a (formerly C2c2), Cas13b, Cas13c, and Cas13d. Cas13s function similarly to Cas9, using a ˜64-nt guide RNA to encode target specificity. The Cas13 protein complexes with the guide RNA via recognition of a short hairpin in the crRNA, and target specificity is encoded by a 28-30-nt spacer that is complementary to the target region. In addition to programmable RNase activity, all Cas13s exhibit collateral activity after recognition and cleavage of a target transcript, leading to non-specific degradation of any nearby transcripts regardless of complementarity to the spacer. Wessels, H.-H. et al. Nature Biotechnol. https://doi.org/10.1038/s41587-020-0456-9 (Published Mar. 16, 2020). In one aspect, the term also includes optimized versions of Cas13 and Cas13 orthologs.

As used herein, the term “pharmaceutical composition” refers to the combination of an active agent with a carrier, inert or active, making the composition especially suitable for therapeutic use in vivo or ex vivo

As used herein, the term “carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, for example, Martin, Remington's Pharmaceutical Sciences, 15th Ed., Mack Publ. Co., Easton, PA [1975].

As used herein, the term “contact” or “contacting” refers to a method of exposure, which can be direct or indirect. In one method, such contact comprises direct injection of the cell through any means well known in the art, such as microinjection. In another method, supply to the cell is indirect, such as via provision in a culture medium that surrounds the cell, or administration to a subject, or via any route known in the art. In another method, the term means that the molecule is introduced into a subject receiving treatment, and the molecule is allowed to come in contact with the cell in vivo. In another method, the term means that a vector or polynucleotide encoding the molecule is supplied to the cell or introduced into a subject receiving treatment, thereby delivering the molecule to a subject.

The term “gRNA” or “guide RNA” as used herein refers to the guide RNA sequences used to target specific genes for correction employing the CRISPR technique. Techniques of designing gRNAs and donor therapeutic polynucleotides for target specificity are well known in the art. See, e.g., Doench et al. (2014) Nature Biotechnol. 32 (12): 1262-7 and Graham et al. (2015) Genome Biol. 16:260, incorporated by reference herein. When used herein, gRNA can refer to a dual or single gRNA.

The term “cell” or “host cell” as used herein may refer to either a prokaryotic or eukaryotic cell, optionally obtained from a subject or a commercially available source. The cell or host cell can be a mammalian cell, e.g., a canine, a feline, a porcine, a rat, a murine, an equine or a human cell. Preferably, the cell can be a mammalian cell. More preferably, the cell can be a human cell. The cell can be from a particular tissue type. Preferably, the cell is a muscle cell.

The term “encode” as it is applied to nucleic acid sequences refer to a polynucleotide which is said to “encode” a polypeptide if, in its native state or when manipulated by methods well known to those skilled in the art, can be transcribed and/or translated to produce the mRNA see Schmok J C, et al. (2024)

nucleic acid, and the encoding sequence can be deduced therefrom.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

As used herein, the term “functional” may be used to modify any molecule, biological, or cellular material to intend that it accomplishes a particular, specified effect.

The term “isolated” as used herein refer to molecules or biologicals or cellular materials being substantially free from other materials.

As used herein, the terms “nucleic acid sequence,” “nucleotide sequence,” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

As used herein, the term “tiling approach” means the process of generating a probe set that contains overlapping probes which are complementary to and span a region of interest in reference sequence and utilizing the probe set to generate DNA segments of the region of interest that may be expressed to generate polypeptide segments that are encoded by the DNA segment of the region of interest. The DNA segments generated using the probe sets can be the same length. The DNA segments generated using the probe sets can be different lengths. The probe sets can be randomly generated based on the reference sequence. The probe sets can be generated based on annotations of the reference sequence to identify possible functional domains. The reference sequence can be a sequence that encodes an RNA-binding protein or a fragment thereof.

As used herein, the term “transfection” or “transfecting” or “transfect” is understood to mean the introduction or process of introducing a nucleic acid or protein into a cell. The nucleic acid can be a foreign nucleic acid or engineered nucleic acid. The protein can be a foreign protein or engineered protein. The protein can be a fusion protein. As well as methods such as electroporation, calcium phosphate precipitation and introduction of the nucleic acid by means of a particle gun, methods wherein the nucleic acid is introduced into the cell by means of a transfection reagent are known in the art today. Methods of protein transfection may include, but are not limited to, use of liposomes, lipid aggregates, nanoparticles, or membrane-disrupting, pore-forming reagents.

As used herein, the term “vector” intends a vector that can express an exogenous polynucleotide. The vector can be a plasmid, or can be derived from or based on a wild-type virus. Aspects of this disclosure relate to an adeno-associated virus, an adenovirus, or lentiviral vector.

A “viral vector” is defined as a recombinantly produced virus or viral particle that comprises a polynucleotide to be delivered into a host cell, either in vivo, ex vivo or in vitro.

Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106 (15): 6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5 (7): 823-827. In aspects where gene transfer is mediated by a retroviral vector, a vector construct refers to the polynucleotide comprising the retroviral genome or part thereof, and a therapeutic gene. Further details as to modern methods of vectors for use in gene transfer may be found in, for example, Kotterman et al. (2015) Viral Vectors for Gene Therapy: Translational and Clinical Outlook Annual Review of Biomedical Engineering 17.

The term “adeno-associated virus” or “AAV” as used herein refers to a member of the class of viruses associated with this name and belonging to the genus dependoparvovirus, family Parvoviridae. Multiple serotypes of this virus are known to be suitable for gene delivery; all known serotypes can infect cells from various tissue types. At least 11, sequentially numbered, are known in the art. Non-limiting exemplary serotypes useful in the methods disclosed herein include any of the 11 serotypes, e.g., AAV2 and AAV8, or variant serotypes, e.g. AAV-DJ.

The term “lentivirus” as used herein refers to a member of the class of viruses associated with this name and belonging to the genus lentivirus, family Retroviridae. While some lentiviruses are known to cause diseases, other lentivirus are known to be suitable for gene delivery. See, e.g., Tomás et al. (2013) Biochemistry, Genetics and Molecular Biology: “Gene Therapy—Tools and Potential Applications,” ISBN 978-953-51-1014-9, DOI: 10.5772/52534.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions can be administered to a subject having one or more signs or symptoms of a disease or condition described herein.

“Treating” or “treatment” as used herein covers the treatment of a disease or disorder described herein, in a subject, such as a human, and includes: (i) relieving a disease or disorder, i.e., causing regression of the disorder; (ii) slowing progression of the disorder; and/or (iii) inhibiting, relieving, or slowing progression of one or more symptoms of the disease or disorder. In some illustrations, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment can be a continuous prolonged treatment for a chronic disease or a single, or few time administrations for the treatment of an acute condition.

As used herein, the term “peptide” refers to a polymer of amino acid residues joined by amide linkages, which can optionally be chemically modified to achieve desired characteristics. The term “amino acid residue,” includes but is not limited to amino acid residues contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also can include unnatural amino acids or residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyricacid, N-Methylglycine, sarcosine, 3-Aminoisobutyric acid, N-Methylisoleucine, 2-Aminopimelicacid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. Typically, the amide linkages of the peptides are formed from an amino group of the backbone of one amino acid and a carboxyl group of the backbone of another amino acid.

The terms “protein,” “peptide,” “polypeptide,” and “amino acid sequence” are used interchangeably herein to refer to polymers of amino acid residues of any length. The polymers can be linear or branched. The polymers can comprise modified amino acids or amino acid analogs and can be interrupted by chemical moieties other than amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling or bioactive component.

As used herein, the terms “subject,” “patient,” or “individual” can be an individual organism, a vertebrate, a mammal, or a human. In some illustrations, the subject, patient or individual is a human.

A “fragment” is a portion of an amino acid sequence or a polynucleotide which is identical in sequence to but shorter in length than a reference sequence. A fragment can comprise up to the entire length of the reference sequence, minus at least one nucleotide/amino acid residue. For example, a fragment can comprise from 5 to 1000 contiguous nucleotides or contiguous amino acid residues of a reference polynucleotide or reference polypeptide, respectively. In some illustrations, a fragment can comprise at least 4, 5, 6, 7, 8, 9, 10, 11, 12, 13, 14, 15, or 16 contiguous amino acid residues of a reference peptide, respectively. Fragments can be preferentially selected from certain regions of a molecule. The term encompasses the full-length polynucleotide or full-length polypeptide.

### High-Throughput Assay to Identify Candidate RNA-Binding Proteins

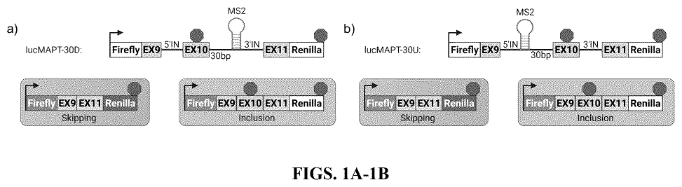

Provided herein is a high-throughput assay to identify candidate RNA-binding proteins for use in developing targeted exon inclusion molecules. In one illustration, the assay is performed using reporters. These reporters can be, but are not limited, to the dual-luciferase reporters shown in FIGS. 1A and 1B.

An RNA-binding protein can be any protein that has the ability to bind to an RNA molecule. The RNA-binding protein can bind to a specific RNA sequence. The RNA-binding protein can bind non-specifically to an RNA molecule regardless of the RNA sequence. The RNA-binding protein can bind to specific secondary structures of an RNA molecule. Testing of RNA-binding proteins in the high throughput assay to determine whether an RNA binding protein may be useful in generating targeted exon inclusion molecules can involve the production of a set of first fusion proteins. The first fusion proteins can include an RNA-binding protein or a fragment thereof. The first fusion proteins can also include a reporter binding domain. The reporter binding domain can be a protein or fragment thereof that is capable of binding to the reporter of the high-throughput assay at a specific reporter binding domain recognition sequence. The reporter binding domain recognition sequence can be a sequence on the reporter mRNA that is recognized by the reporter binding domain. The reporter binding domain may recognize the secondary structure of the reporter binding domain recognition sequence. The reporter binding domain may recognize the specific sequence of the reporter binding domain recognition sequence. The reporter binding domain can be fused to the C-terminus of the RNA-binding protein. The reporter binding domain can be fused to the N-terminus of the RNA-binding protein.

The reporter binding domain portion of the RNA-binding protein/reporter binding domain fusions can be recruited to the reporter at the reporter binding domain recognition sequence to allow the RNA-binding protein to come into proximity with the reporter and bind to the reporter as well to direct inclusion of a target exon. The reporter binding domain recognition sequence is adjacent to the target exon splice site. The reporter binding domain recognition sequence can be at least 50, 45, 40, 35, 30, 25, 20, 15, 10, or 5 nucleotides upstream of the target exon splice site. The reporter binding domain recognition sequence can be at least 50, 45, 40, 35, 30, 25, 20, 15, 10, or 5 nucleotides downstream of the target exon splice site.

The reporter binding domain can be, but is not limited to, an MS2 coat protein. The reporter binding domain recognition sequence can be, but is not limited to, the MS2-stem loop RNA sequence. The MS2-stem loop RNA sequence can be used to recruit the MS2 coat protein region of the first fusion proteins to the reporter for targeted positioning of the first fusions either upstream or downstream of the target exon splice site. Other reporter binding domain recognition sequences that recruit other reporter binding domains can be used in the reporter to direct binding of the first fusion proteins upstream or downstream of the target exon splice site. Non-limiting examples of other reporter binding domains and reporter binding domain recognition sequences include PCP and PP7-stem loop.

The reporters can be used to test target exon inclusion upon binding of the RNA-binding protein or a fragment thereof downstream of the target exon splice site. The reporters can be used to test target exon inclusion upon binding of the RNA-binding protein or a fragment thereof upstream of the target exon splice site. The downstream inclusion reporters can include, from 5′ to 3′, a first reporter gene, a target exon, a reporter binding domain recognition sequence, and a second reporter gene. The upstream inclusion reporter can include, from 5′ to 3′, a first reporter gene, a reporter binding domain recognition sequence, a target exon, and a second reporter gene. The first reporter gene and the second reporter gene can be from different reporter genes. The first reporter gene and second reporter gene can be luciferase genes. The luciferase genes of the first reporter gene and the second reporter genes can be from different species and can have different emission signal wavelengths. The first reporter gene can be the firefly luciferase gene. The second reporter gene can be the Renilla luciferase gene.

The target exon can contain an in-frame stop codon, wherein inclusion of the target exon in the final mRNA product would lead to termination of transcription before the second reporter gene is transcribed. Termination of transcription before the second reporter gene has been transcribed results in decreased expression of the second reporter while expression of the first reporter is continuously expressed regardless of whether the target exon is included or excluded from the final mRNA product.

Generally, the alternatively spliced target exon in the reporter is included in the final mRNA at very low levels in the absence of positive modulators. Thus, increased inclusion of the alternatively spliced target exon leads to a higher proportion of final reporter mRNA molecules containing a stop codon before the reading frame of the second reporter gene than when there is not increased inclusion of the target exon.

Measuring the presence of alternatively spliced reporter molecules can be done by measuring the amount of the final reporter mRNA molecules containing the target exon. When the first reporter and second reporters are fluorescent molecules, measuring the expression levels of the first reporter and the second reporter can be done by measuring the intensity of the fluorescence using, for example, fluorescence microscopy or fluorescence spectroscopy. When the first fusion protein is capable of directing target exon inclusion during splicing of the reporter, the stop codon incorporated into the final reporter mRNA molecule will result in termination of translation of the mRNA molecule into a polypeptide product before the second reporter gene is translated, thus generating polypeptide products containing only the first reporter and not the second reporter. The production of polypeptide products containing only the first reporter and not the second reporter results in a reduction in the ratio of expression of the second reporter to expression of the first reporter as compared to a first fusion protein that includes an protein or a fragment thereof that is derived from a protein that cannot bind RNA or binds RNA less efficiently or effectively due to the fusion proteins' relative abilities to direct target exon inclusion during splicing. Measuring the presence of the alternatively spliced reporter molecules can be done by measuring the amount of protein expressed from the final reporter mRNA molecules containing the target exon or by measuring the amount of final reporter mRNA molecules containing the target exon. In some illustrations, measuring the presence of the alternatively spliced reporter molecules can be done by measuring the expression levels of the first reporter and the second reporter. In some illustrations, measuring the presence of the alternatively spliced reporter protein molecules can be done by western blot or other methods known in the art. In some illustrations, measuring the presence of the alternatively spliced reporter mRNA molecules can be done by Northern blot, quantitative real-time reverse transcription PCR (qRT-PCR), or other methods known in the art.

An RNA-binding protein can be identified as a candidate for development into a targeted exon inclusion molecule if transfection with the first fusion and the reporter results in a decreased ratio of second reporter gene expression to first reporter gene expression than transfection with a control reporter binding protein fusion and the reporter. Promising candidates for developing targeted exon inclusion molecules are the RNA-binding proteins of RNA-binding protein/reporter binding domain fusions that caused a statistically significant decrease in the ration of expression of the second reporter gene to expression of the first reporter gene. A first fusion protein may be considered to effectively direct targeted exon inclusion when there is a statistically significant reduction in the ratio of expression of the second reporter to expression of the first reporter as compared to the ratio seen for a fusion protein that includes a protein known to not bind to RNA. The protein known to not bind to RNA can be protein tag. The protein tag can be a peptide sequence that do not bind to RNA. The protein tag can be a FLAG-tag. Without being bound by the hypothesis, it is thought that the RNA-binding proteins may direct targeted exon inclusion by acting through spliceosomal recruitment. Endogenously, the identified proteins contain domains that direct them to the targets for modulated exon inclusion as well as protein-protein interaction domains that facilitate interactions with other splicing factors or components of the spliceosomal complex. The combination of effects leads to recruitment of these factors to regulatory regions of target RNA molecules, enhancing spliceosomal assembly and facilitating exon inclusion.

### Development of Targeted Exon Inclusion Molecules

Provided herein are methods for developing targeted exon inclusion molecules and examples of such. A targeted exon inclusion molecule can be a molecule that can bind to RNA and, upon binding, direct alternative splicing of the RNA molecule to include a target exon in the final processed RNA molecule. Each targeted exon inclusion molecule can include an RNA-binding protein or an optimized effector domain of an RNA-binding protein and an RNA-targeting moiety. The optimized effector domain can be an RNA-binding protein or a fragment thereof. The optimized effector domain can be selected by screening smaller fragments of the RNA-binding proteins or fragments thereof identified in the high-throughput assay. The optimized effector domain can comprise a minimal effector domain of the identified RNA-binding protein or fragment thereof. The targeted exon inclusion molecule can be a polypeptide. The polypeptide of the targeted exon inclusion molecule can be encoded by a polynucleotide sequence. The polynucleotide sequence encoding the targeted exon inclusion molecule can be a DNA sequence or an RNA sequence. The polynucleotide sequence encoding the targeted exon inclusion molecule can be included in a plasmid or a vector.

### RNA-Binding Proteins, Domains, and Minimal Effector Domains

RNA-binding proteins, cither full length proteins or fragments thereof, identified in the high-throughput assay, or thought to have an effect on directing targeted exon inclusion, can be utilized to generate targeted exon inclusion molecules as the optimized effector domain of the targeted exon inclusion molecule.

In other illustrations, smaller fragments of the RNA-binding protein can be identified by annotating the RNA-binding protein of the tested first fusion protein to identify functional domains that are predicted bind to RNA. One method of testing the domains of the RNA-binding proteins is to generate second fusion proteins including the identified domains of the RNA-binding proteins and the reporter binding protein and subjecting the generated second fusion proteins to the high-throughput assay described above. Domains that are effective for directed targeted exon inclusion can be identified by a decrease in the ratio of expression of the second reporter gene to expression of the first reporter gene as compared to a fusion protein that includes an RNA-binding domain that is known to not bind RNA. The identified domains of the RNA-binding protein can be included in a targeted exon inclusion molecule as the optimized effector domain.

In further illustrations, the identified domains from the second fusion proteins can be further tested to identify a minimal effector domain. One method of identifying the minimal effector domain of the identified domains of RNA-binding proteins is by subcloning the identified domains in a tiling approach. The tiling approach can involve the use of polypeptide fragments that span the entire length of the identified domain. The polypeptide fragments can vary in length. The polypeptide fragments can be randomly generated. The polypeptide fragments can be generated based on possible RNA binding effect of the polypeptide fragments. Each polypeptide fragment can be fused to the reporter binding protein to generate third fusion proteins; the third fusion proteins can then be subjected to the high-throughput assay described above. The polypeptide fragments that are effective for directing targeted exon inclusion can be included in a targeted exon inclusion molecule. The polypeptide fragments can be further subcloned to identify smaller portions of the fragment that can be capable of directing targeted exon inclusion by repeating the process described above. The smallest portion of the fragment of the identified domains of the RNA-binding proteins can be termed a minimal effector domain. The minimal effector domain can be included in a targeted exon inclusion molecule.

### RNA-Targeting Moieties

An RNA-targeting moiety is a moiety that can be recruited to specific sequences of an RNA molecule. These can be used to direct the targeted exon inclusion molecule to a specific mRNA sequence to direct inclusion of a specific target exon. The RNA-targeting moieties can include, but are not limited to, Cas13 systems, CRISPR-Cas-inspired RNA targeting systems (CIRTS), pumilio (PUF) RNA binding proteins, antisense oligonucleotides, and small molecule compounds. It is to be understood that other proteins that can bind to specific RNA sequences can be utilized as an RNA-targeting moiety.

Cas13 systems can include a protein component, which is the Cas protein, and a guide RNA. The guide RNA can mediate RNA target selection. The guide RNA can include two key regions: a direct repeat, which associates with the Cas protein, and a spacer sequence, which is antisense to the target RNA sequence and can bind to the target RNA sequence. The Cas protein of the Cas13 system can be fused to the C-terminus or the N-terminus of the optimized effector domain of the targeted exon inclusion molecule. Prior art Cas13 systems are known in the art, e.g., see U.S. Pat. No. 11,739,308 and U.S. patents application Ser. Nos. 16/631,879 and 16/649/170.

CIRTS can be RNA-targeting systems engineered to consist completely of or essentially completely of human-derived protein to evade immunogenicity concerns of standard Cas13 systems. CIRTS proteins can be designed to mimic the structure and function of Cas13. CIRTS systems can include a protein component, which is the human-derived protein mimic of the Cas protein, and a guide RNA. The guide RNA can mediate RNA target selection. The guide RNA can include two key regions: a direct repeat, which associates with the CIRTS protein, and a spacer sequence which is antisense to the target RNA sequence and can bind to the target RNA sequence. The CIRTS protein of the CIRTS system can be fused to the C-terminus or the N-terminus of the optimized effector domain of the targeted exon inclusion molecule. Prior art CIRTS are known in the art, e.g., U.S. patent application Ser. No. 17/309,936.

PUF RNA binding proteins are human-derived RNA-targeting systems inspired by the naturally occurring PUF family of proteins. PUF proteins can contain the Pumilio-homology domain (PumHD), which targets a specific RNA sequence through a chain of modules that bind to each specific RNA base. It is possible to tune PUF proteins to target specific sequences by changing the amino acid composition at three key residues. PUFs can be single-component RNA-targeting systems and not require the co-expression with a guide RNA. The PUF protein can be fused to the C-terminus or the N-terminus of the optimized effector domain of the targeted exon inclusion molecule.

ASOs can be single-stranded DNA molecules that target RNA transcripts through antisense interactions. ASOs can be used to recruit endogenous human proteins to targeted RNA transcripts. ASOs can be engineered to be antisense to the target RNA sequence and bind to the target RNA sequence. The ASO can be fused to the C-terminus or the N-terminus of the optimized effector domain of the targeted exon inclusion molecule. The ASO can bind to the target RNA sequence and promote recruitment of the targeted exon inclusion molecule based on the heteroduplex of the ASO-target mRNA. The ASO and RNA-binding protein fusion may be encapsulated in a packaging material, such as a non-virus-like particle, for delivery into cells or to a subject.

Small molecule compounds can be engineered to recruit proteins to three-dimensional structures on RNA targets. The small molecule compounds can be engineered to recruit the proteins to specific target RNA sequences.

The RNA-targeting moieties can be capable of binding to or recruiting the target mRNA to the targeted exon inclusion molecule. The RNA-targeting moiety can be capable of binding to or recognizing a target RNA sequence of the target mRNA. The target RNA sequence can be adjacent to the target exon splice site. The target RNA sequence can be at least 50, 45, 40, 35, 30, 25, 20, 15, 10, or 5 nucleotides upstream of the target exon splice site. The target RNA sequence can be at least 50, 45, 40, 35, 30, 25, 20, 15, 10, or 5 nucleotides downstream of the target exon splice site. Recruitment and/or binding of the target RNA sequence of the target mRNA by the targeted exon inclusion molecule allows the optimized effector domain of the molecule to come into proximity to the mRNA and further bind the mRNA.

The RNA-targeting moieties can be programmed for any target and thus can be rapidly applied following identification of diseases amenable to splicing regulation. Similar approaches have been used to apply antisense oligonucleotides (ASOs) in personalized therapeutic applications, and this approach provides a competitive method with potentially greater strength in cases where increased exon inclusion is more therapeutically appropriate.

### Compositions Utilizing the Targeted Exon Inclusion Molecules

Provided herein are compositions utilizing the targeted exon inclusion molecules described above. The compositions described herein can be used to direct inclusion of target exons in target mRNAs and can include the targeted exon inclusion molecules described above.

The compositions can include a carrier. The carrier can be a pharmaceutically acceptable carrier. Pharmaceutically acceptable carriers for various dosage forms are known in the art. For example, excipients, lubricants, binders, and disintegrants for solid preparations are known; solvent, solubilizing agents, suspending agents, isotonicity agents, buffers, and soothing agents for liquid preparations are known. The compositions can include one or more additional components, such as one or more preservatives, antioxidants, colorants, sweetening/flavoring agents, absorbing agents, wetting agents, and the like. The composition can further include a guide RNA if necessary for the RNA-targeting moiety to be recruited to the target mRNA.

The compositions can include the polynucleotide encoding the targeted exon inclusion molecule. The compositions can further include a carrier, more preferably a pharmaceutically acceptable carrier. The compositions can further include a guide RNA if necessary for the RNA-targeting moiety to be recruited to the target mRNA.

The compositions can include a vector including the polynucleotide encoding the targeted exon inclusion molecule. The vector can be, but is not limited to, an adeno-associated viral (AAV) vector, a retroviral vector, a lentiviral vector, or a herpes simplex viral vector. The composition can further include a guide RNA if necessary for the RNA-targeting moiety to be recruited to the target mRNA. The vector can also include the sequence of a guide RNA if the guide RNA is necessary for the RNA-targeting moiety of the targeted exon inclusion molecule to be recruited to the target mRNA.

The compositions can further comprise any other reagent or component sufficient, necessary, or useful for practicing any of the methods described herein. Such reagents or components include, but are not limited to, transfection reagents, culture medium (e.g., MEF-condition medium), cells (e.g., somatic cells, iPS cells), containers, boxes, buffers, inhibitors (e.g., RNase inhibitors), labels (e.g., fluorescent, luminescent, radioactive, etc.), positive and/or negative control molecules, reagents for generating capped mRNA, dry ice or other refrigerants, instructions for use, cell culture equipment, detection/analysis equipment, and the like.

### Methods of Using Targeted Exon Inclusion Molecules

Provided herein are also method for using the targeted exon inclusion molecules. The methods can be directed to in vitro and in vivo applications of the targeted exon inclusion molecules.

A method of utilizing the targeted exon inclusion molecules includes contacting a cell including an mRNA target with the targeted exon inclusion molecules under conditions to allow the targeted exon inclusion molecule to bind to the target RNA sequence of the target mRNA and facilitate inclusion of a target exon during splicing of the mRNA. The conditions can include contacting the cells with a guide RNA that allows the RNA-targeting moiety of the targeted exon inclusion molecule to be recruited to the target mRNA. The contacting of the cell can occur in vitro or in vivo.

The cell can be a plant cell, a prokaryotic cell, or a eukaryotic cell. The cell can preferably be a mammalian cell. The cell can more preferably be a human cell. The cell can be from a specific tissue or cell type. The cell can be a human muscle cell, a human neuron, or a human lung cell. The cell can be from a subject with a disease or disorder associated with mis-splicing of an mRNA. The disease or disorder can be muscular dystrophy, spinal muscular atrophy (SMA), Alzheimer's disease, familial dysautonomia, early-onset Parkinson's disease, X-linked parkinsonism with spasticity, cystic fibrosis, or CDKL5-deficiency disorder.

The method can further comprise measuring the expression of the polynucleotide including the target exon. Measuring expression of the polynucleotide comprising the target exon can be done by sequencing the target mRNA in the cell, sequencing the cDNA derived from the target mRNA in the cell, or by analyzing the polypeptides or proteins produced in the cell from the target mRNA including the target exon. Measuring the presence of the alternatively spliced target mRNA molecules can be done by Northern blot, quantitative real-time reverse transcription PCR (qRT-PCR), or other methods known in the art. The amount of the polynucleotide of the target mRNA including the target exon can be compared to the amount of the polynucleotide of the target mRNA that does not include the target exon. The amount of the polypeptides or proteins produced in the cell from the target mRNAs including the target exon can be compared to the amount of the polypeptides or proteins produced in the cell from the target mRNAs that do not include the target exon. Measuring the presence of the alternatively spliced target protein molecules can be done by western blot or other methods known in the art.

One in vitro method can include contacting human muscle cells with the targeted exon inclusion molecules under conditions to allow the targeted exon inclusion molecule to bind to the target RNA sequence of the target mRNA and facilitate inclusion of a target exon during splicing of the target mRNA. The human muscle cells can be collected or derived from a subject having muscular dystrophy. The target mRNA can be a pre-mRNA encoding the dystrophin gene or a functional fragment thereof. The method can further comprise measuring the expression of the dystrophin gene and measuring the levels of dystrophin protein produced from mRNA that include the target exon of the dystrophin gene.

### Treatments

The methods disclosed herein can be used to treat a disease or disorder in a subject having the same. One method of treating a subject having a disease with the targeted exon inclusion molecule can include administering to the subject a therapeutically-effective amount of the targeted exon inclusion molecule. The method can require administration of one, two, three, four, five, six, seven, eight, nine, ten, or more doses of the target exon inclusion molecule. The method of administration can be locally or systemically, as determined by the treating physician, or intramuscularly or intravenously.

One such treatment method can include administering to a subject having muscular dystrophy a therapeutically effective amount of the target exon inclusion molecule or composition including the target exon inclusion molecule under the conditions to allow the targeted exon inclusion molecule to bind to the target RNA sequence of the target mRNA and facilitate inclusion of a target exon during splicing of the target mRNA. Muscular dystrophy is a group of muscle diseases that results in muscle weakness and loss of skeletal muscles. Muscular dystrophy is associated with genetic mutations in various genes. One form of muscular dystrophy is Becker muscular dystrophy (BMD) which is caused by a mutation on the X chromosome in the dystrophin gene. The dystrophin protein is a cytoskeletal protein that is located at the cell membranes of muscle tissues and are integral for maintaining muscle membrane stability; it is also associated with several signaling pathways, such as the Ras/mitogen-activated protein kinase (MAPK) pathway and nitric oxide synthesis. Mutations in the dystrophin gene, including mutations that increase exon skipping, that are associated with BMD typically result in truncated forms of the dystrophin protein, which results in increased fragility of the sarcolemma or cell membrane surrounding the skeletal muscle fiber and persistent immune cell infiltration and fibrosis. Thus, utilizing the targeted exon inclusion molecule can present a method of treating BMD by directing inclusion of exons typically skipped due to a genetic mutation in the dystrophin gene. The target mRNA can be a pre-mRNA encoding dystrophin or a functional fragment thereof. The targeted exon inclusion molecule can include an RNA-targeting moiety that is generated to specifically target a region of the dystrophin pre-mRNA adjacent to the target exon. The targeted exon inclusion molecule can direct inclusion of the target exon following binding of the targeted exon inclusion molecule by the optimized effector domain. This inclusion of the target exon by the targeted exon inclusion molecule can result in production of full-length dystrophin protein or a functional fragment of the dystrophin protein that can act to stabilize the sarcolemma and/or operate in the various signaling processes from mRNA molecules that include the target exon after splicing of the pre-mRNA. The production of full-length dystrophin can result in increased stabilization of the skeletal muscle sarcolemma and reduction in immune cell infiltration the skeletal muscle and fibrosis and can result in reduced degeneration of the muscle tissue.

Another such treatment method can include administering to a subject having SMA a therapeutically effective amount of the target exon inclusion molecule or composition including the target exon inclusion molecule under the conditions to allow the target exon inclusion molecule to bind to the target RNA sequence of SMN2 mRNA and facilitate inclusion of exon 7 during splicing of SMN2 mRNA. Another treatment method can include administering to a subject having Alzheimer's disease a therapeutically effective amount of the target exon inclusion molecule or composition including the target exon inclusion molecule under the conditions to allow the target exon inclusion molecule to bind to the target RNA sequence of LRP8/ApoER2 mRNA and facilitate inclusion of exon 19 during splicing of the LRP8/ApoER2 mRNA. Another such treatment method can include administering to a subject having familial dysautonomia a therapeutically effective amount of the target exon inclusion molecule or composition including the target exon inclusion molecule under the conditions to allow the target exon inclusion molecule to bind to the target RNA sequence of IKBKAP/ELP1 mRNA and facilitate inclusion of exon 20 during splicing of the IKBKAP/ELP1 mRNA. Another such treatment method can include administering to a subject having early-onset Parkinson's disease a therapeutically effective amount of the target exon inclusion molecule or composition including the target exon inclusion molecule to bind to the target RNA sequence of PINKI mRNA and facilitate inclusion of exon 7 during splicing of PINKI mRNA. Another such treatment method can include administering to a subject having X-linked parkinsonism with spasticity a therapeutically effective amount of the target exon inclusion molecule or composition including the target exon inclusion molecule to bind to the target RNA sequence of ATP6AP2 mRNA and facilitate inclusion of exon 4 during splicing of ATP6AP2 mRNA. Another such treatment method can include administering to a subject having cystic fibrosis a therapeutically effective amount of the target exon inclusion molecule or composition including the target exon inclusion molecule to bind to the target RNA sequence of CFTR mRNA and facilitate inclusion of exon 13 or exon 16 during splicing of CFTR mRNA to increase production of functional CFTR protein. Another such treatment method can include administering to a subject having CDKL5-deficiency disorder a therapeutically effective amount of the target exon inclusion molecule or composition including the target exon inclusion molecule to bind to the target RNA sequence of CDKL5 mRNA and facilitate inclusion of exon 3 during splicing of CDKL5 mRNA to rescue functional CDKL5 protein production.

The method of treatment may be used to treat other diseases or disorders including spinal muscular atrophy (SMA), Alzheimer's disease, familial dysautonomia, early-onset Parkinson's disease, X-linked parkinsonism with spasticity, cystic fibrosis, or CDKL5-deficiency disorder. These methods of treatment can include targeting of exons in mRNA transcribed from genes associated with the disease or disorder selected for treatment. The methods of treatment can include generation of targeted exon inclusion molecules that include RNA-targeting moieties designed to target the mRNA underlying the disease or disorder selected for treatment near the target exon. A person of skill in the art would understand what target exon and target mRNA would be utilized for the selected disease or disorder based on the disclosure of the prior art.

### Diagnostic Applications

The targeted exon inclusion molecules may be used to investigate alternative splicing vents that contribute to disease development and progression. This can be done by screening for potential splicing events of interest by driving increased inclusion and measuring resulting phenotypes.

### EXAMPLES

### Example No. 1

### Exemplary High-Throughput Screen

The high-throughput assay was performed using reporters shown in FIGS. 1A and 1B. A dual-luciferase reporter was co-transfected with an RNA-binding protein of interest used at the C-terminal to the MS2 bacteriophage coat protein using Lipofectamine 3000. The MS2-stem loop RNA structure in the reporter recruits the MS2 coat protein in the RNA-binding protein fusion for targeted positioning. The alternatively spliced exon in the reporter is included at very low levels in the absence of positive modulators. Increased inclusion of the alternatively spliced exon leads to a higher proportion of final mRNA molecules containing a stop codon before the reading frame of Renilla luciferase, and therefore a decrease in the Renilla:firefly ratio. Two reporters were developed, one containing the MS2 stem loop 30 bases 3′ of the downstream 5′ splice site (lucMAPT-30D) and one containing the MS2 stem loop 30 bases 5′ of the upstream 3′ splice site (lucMAPT-30U).

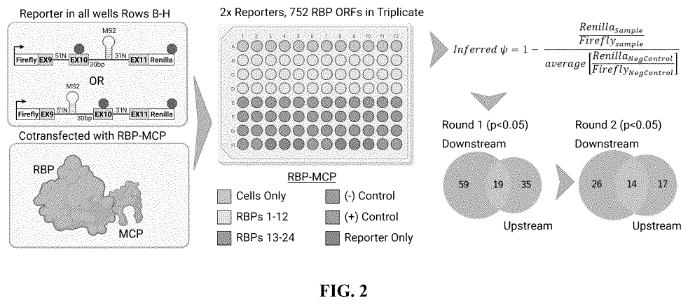

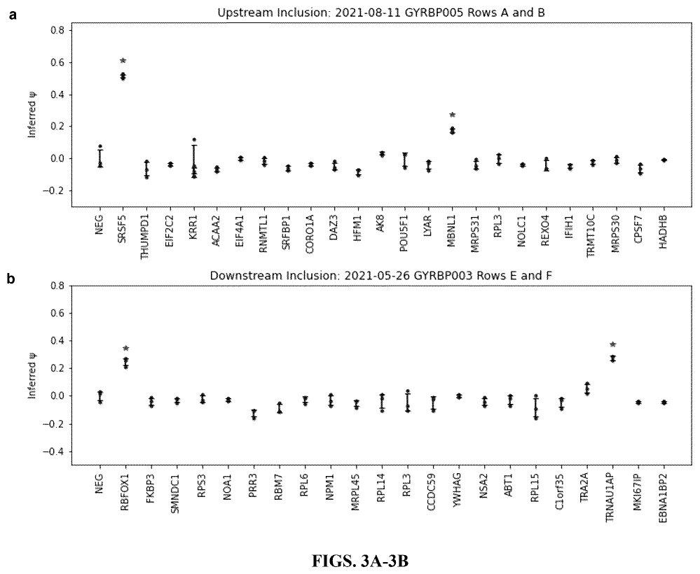

A screen of 752 RNA-binding proteins fused to the MS2 coat protein was performed using both reporters. All candidates were tested with three replicates. Exemplary data from a 96-well plate is shown in FIG. 3. The experimental schematic is shown in FIG. 2. Candidates causing a significant decrease in the Renilla:firefly ratio (p<0.05) were kept for a second round of testing with the reporter.

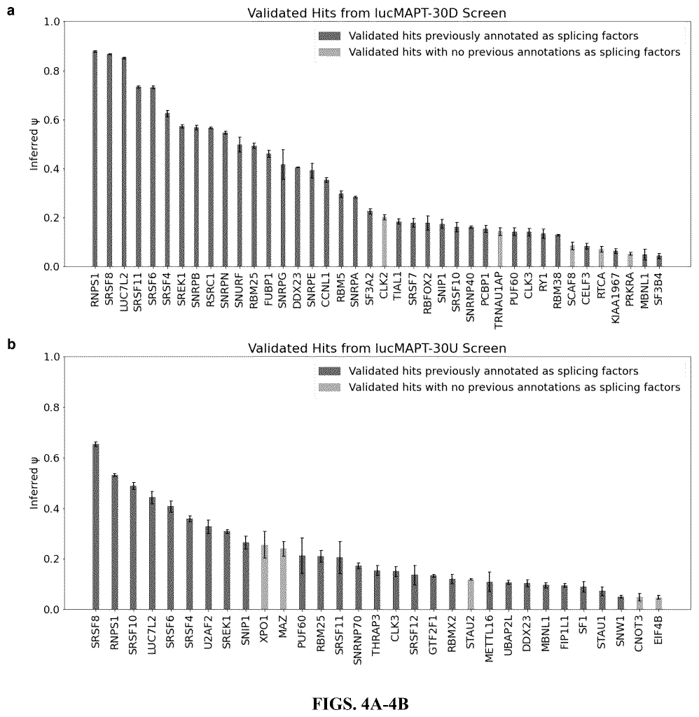

Candidates that caused a significant decrease in the Renilla:firefly ratio (p<0.05) in the second round of testing comprise the final list of hits (FIG. 4). The list of candidates include: SRSF8, RNPS1, SRSF10, SRSF4, SRSF5, SREK1, LUC7L2, SRSF6, SNIP1, U2AF2, GTF2F1, RBM25, STAU2, MAZ, CLK3, THRAP3, FIL1L1, MBNL1, SNRNP70, DDX23, XPO1, UBAP2L, SRSF12, RBMX2, SRSF11, PUF60, SNW1, METTL16, SF1, STAU1, CNOT3, EIF4B, SNRPN, SNRPB, SNRPA, RBM5, SNRNP40, RSRC1, TIAL1, FUBP1, SNRUF, SRSF7, TRNAU1AP, CCNL1, SNRPE, RBFOX1, RBFOX2, KIAA1967, SNRPG, RTCA, CLK2, PRKRA, SCAF8, SF3A2, PCBP1, SF3B4, RBM38, RY1, and CELF3.

Several candidate RNA-binding proteins, including TRNAU1AP, LUC7L2, SRSF8, SNRPB, FUBP1, U2AF2, and SRSF10, were additionally validated in vitro when fused to a catalytically dead Cas13d. The mRNA target used for the experiment was the luciferase-based splicing reporter used in the identification assay that lacks the MCP/MS2-stem loop recruitment systems. The RNA-binding proteins exhibited exon inclusion when used to the dCas13d domain as well, indicating that the RNA-binding proteins identified via the screen exhibited exon inclusion regardless of the RNA-targeting moiety utilized to confer target RNA specificity.

TABLE 1
Candidates Identified In Exemplary High-Throughput Screen

| | NCBI | |
|---|---|---|
| Symbol | Accession | Full Name |
| SRSF8 | BC057783.1 | serine and arginine rich splicing factor 8 |
| RNPS1 | BC001659.2 | RNA binding protein with serine rich |
| | | domain 1 |
| SRSF10 | BC005039.1 | serine and arginine rich splicing factor |
| | | 10 |
| SRSF4 | BC002781.2 | serine and arginine rich splicing factor 4 |
| SRSF5 | BC018823.2 | serine and arginine rich splicing factor 5 |
| SREK1 | BC067770.1, | splicing regulatory glutamic acid and |
| | BC112343.1 | lysine rich protein 1 |
| LUC7L2 | BC017163.2, | LUC7 like 2, pre-mRNA splicing factor |
| | BC050708.2, | |
| | BC056886.1 | |
| SRSF6 | BC006832.2 | serine and arginine rich splicing factor 6 |
| SNIP1 | BC027040.1 | Smad nuclear interacting protein 1 |
| U2AF2 | BC008740.2 | U2 small nuclear RNA auxiliary factor 2 |
| GTF2F1 | BC000120.1 | general transcription factor IIF subunit 1 |
| RBM25 | BC136775.1 | RNA binding motif protein 25 |
| STAU2 | BC110447.1 | staufen double-stranded RNA binding |
| | | protein 2 |
| MAZ | BC041629.1 | MYC associated zinc finger protein |
| CLK3 | BC019881.1 | CDC like kinase 3 |
| THRAP3 | BC112330.1 | thyroid hormone receptor associated |
| | | protein 3 |
| FIP1L1 | AL136910 | factor interacting with PAPOLA and |
| | | CPSF1 |
| MBNL1 | BC043493.1 | muscleblind like splicing regulator 1 |
| SNRNP70 | BC001315.1 | small nuclear ribonucleoprotein U1 |
| | | subunit 70 |
| DDX23 | BC002366.2 | DEAD-box helicase 23 |
| XPO1 | BC032847.2 | exportin 1 |
| UBAP2L | BC003170.1 | ubiquitin associated protein 2 like |
| SRSF12 | BC021715.1 | serine and arginine rich splicing factor |
| | | 12 |
| RBMX2 | BC033750.1 | RNA binding motif protein X-linked 2 |
| SRSF11 | BC040436.1 | serine and arginine rich splicing factor |
| | | 11 |
| PUF60 | BC009734.1 | poly(U) binding splicing factor 60 |
| SNW1 | BC108903.1 | SNW domain containing 1 |
| METTL16 | BC050603.1 | methyltransferase 16, N6- |
| | | methyladenosine |
| SF1 | BC008080.2, | splicing factor 1 |
| | BC020217.1 | |
| STAU1 | BC050432.1 | staufen double-stranded RNA binding |
| | | protein 1 |
| CNOT3 | BC016474 | CCR4-NOT transcription complex |
| | | subunit 3 |
| EIF4B | BC073139.1 | eukaryotic translation initiation factor |
| | | 4B |
| SNRPN | BC003180.1, | small nuclear ribonucleoprotein |
| | BC010057.1, | polypeptide N |
| | BC024777.1, | |
| | BC025178.1 | |
| SNRPB | BC080516.1 | small nuclear ribonucleoprotein |
| | | polypeptides B and B1 |
| SNRPA | BC000405.2, | small nuclear ribonucleoprotein |
| | BC008290.1 | polypeptide A |
| RBM5 | BC002957.1 | RNA binding motif protein 5 |
| SNRNP40 | BC001494.2 | small nuclear ribonucleoprotein U5 |
| | | subunit 40 |
| RSRC1 | HQ448170 | arginine and serine rich coiled-coil 1 |
| TIAL1 | BC030025.1 | TIA1 cytotoxic granule associated RNA |
| | | binding protein like 1 |
| FUBP1 | BC017247 | far upstream element binding protein 1 |
| SNURF | BC024777.1 | SNRPN upstream open reading frame |
| SRSF7 | BC000997.2, | serine and arginine rich splicing factor 7 |
| | BC017369.2, | |
| | BC022328.1 | |
| TRNAU1AP | BC000680.2 | tRNA selenocysteine 1 associated |
| | | protein 1 |
| CCNL1 | JF432881 | cyclin L1 |
| SNRPE | BC002639.2 | small nuclear ribonucleoprotein |
| | | polypeptide E |
| RBFOX1 | BC113691.1 | RNA binding fox-1 homolog 1 |
| RBFOX2 | BC025281.1 | RNA binding fox-1 homolog 2 |
| CCAR2/ | BC018269.1 | Cell Cycle and Apoptosis Regulator 2 |
| KIAA1967 | | |
| SNRPG | BC000070.2, | small nuclear ribonucleoprotein |
| | BC022432.1, | polypeptide G |
| | BC066302.1 | |
| RTCA | BC012604.1 | RNA 3'-terminal phosphate cyclase |
| CLK2 | BC014067.2 | CDC like kinase 2 |
| PRKRA | BC009470.1 | protein activator of interferon induced |
| | | protein kinase EIF2AK2 |
| SCAF8 | BC070071.1 | SR-related CTD associated factor 8 |
| SF3A2 | BC004434 | splicing factor 3a subunit 2 |
| PCBP1 | BC039742.1 | poly(rC) binding protein 1 |
| SF3B4 | BC004273.1, | splicing factor 3b subunit 4 |
| | BC013886.2 | |
| RBM38 | BC018711 | RNA binding motif protein 38 |
| SNRNP27/ | BC017890.1 | Small nuclear ribonucleoprotein |
| RY1 | | U4/U6.U5 subunit 27 |
| CELF3 | BC052491.1 | CUGBP Elav-like family member 3 |

### Analysis of Candidate RNA-Binding Proteins and Domains

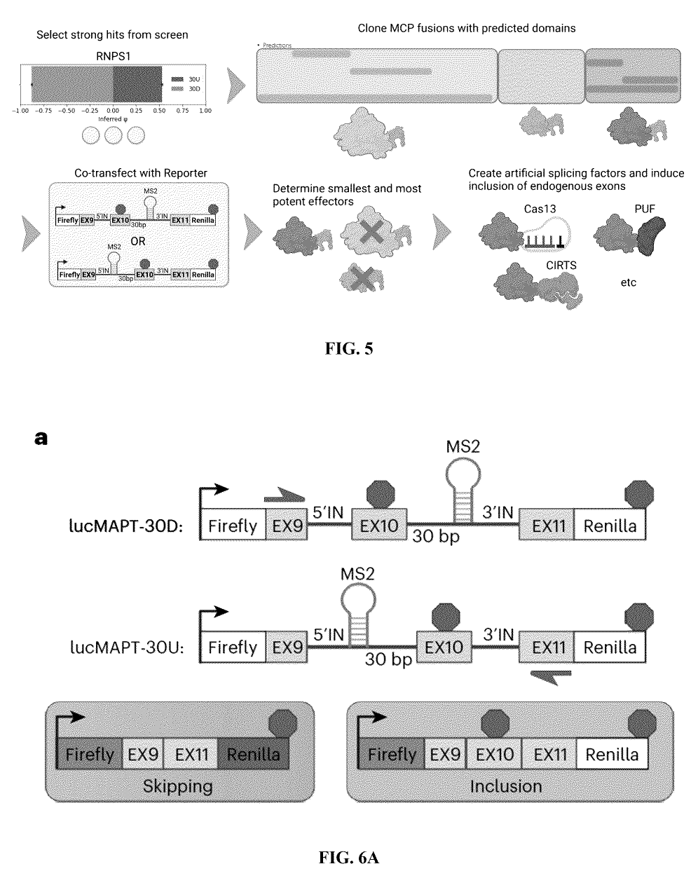

The strongest RNA-binding proteins that cause targeted exon inclusion were investigated further to determine which protein domains are responsible for the effect (FIG. 5). The final candidates identified in the high-throughput screen include some proteins that had known RNA binding activity based on associated gene ontology (GO) terms, while other proteins are not previously associated with any RNA-binding protein activity (FIG. 4, known RNA-binding proteins in blue, novel RNA-binding proteins in yellow). The strongest effector proteins were separated into domains using InterPro annotations. Each domain was, in turn, fused to the MS2 coat protein in a follow-up library. Each new fusions protein was tested with the same assay system described above to determine which domains are able to direct inclusion of the target exon in the reporter and would thus be considered optimized effector domains.

Optimized effector domains identified that retained strong targeted exon inclusion were then further analyzed and investigated to identify minimal effector domains from each. Each optimized effector domain was subcloned using a tiling approach to cover the entire length of the domain. Each subcloned portion of the optimized effector domain was fused to the MS2 coat protein in a second follow-up library and tested in the same assay system above to determine which portions were effective for driving targeted exon inclusion.

Optimized effector domains have been identified from several RNA-binding proteins, including TRNAU1AP, LUC7L2, SRSF8, SNRPB, FUBP1, U2AF2, and SRSF10, as being effective in directing targeted exon inclusion when fused to an RNA-targeting moiety. The specific domains are listed in Table 2.

TABLE 2
Domain Sequences for Candidate RNA-Binding
Proteins.

| Fragment | |
|---|---|
| Name | Sequence |
| TRNAU1AP-4 | GKQPDNSPEYSLFVGDLTPDVDDGMLYEFFVKVY |
| | PSCRGGKVVLDQTGVSKGYGFVKFTDELEQKRAL |
| | TECQGAVGLGSKPVRLSVAIPKASRVKPVEYSQM |
| | YSYSYNQYYQQYQNYYAQWGYDQNTGSYSYSYP |
| | QYGYTQSTMQTYEEVGDDALEDPMPQLDVTEAN |
| | KEFMEQSEELYDALMDCHWQPLDTVSSEIPAMM |
| TRNAU1AP-5 | ASRVKPVEYSQMYSYSYNQYYQQYQNYYAQWG |
| | YDQNTGSYSYSYPQYGYTQSTMQTYEEVGDDALE |
| | DPMPQLDVTEANKEFMEQSEELYDALMDCHWQP |
| | LDTVSSEIPAMM |
| LUC7L2-4 | KQEKRNQERLKRREEREREEREKLRRSRSHSKNPK |
| | RSRSREHRRHRSRSMSRERKRRTRSKSREKRHRHR |
| | SRSSSRSRSRSHQRSRHSSRDRSRERSKRRSSKERF |
| | RDQDLASCDRDRSSRDRSPRDRDRKDKKRSYESA |
| | NGRSEDRRSSEEREAGEI |
| SRSF8-2 | PRSRQGEPRGRSRGGGYGRRSRSPRRRHRSRSRGP |
| | SCSRSRSRSRYRGSRYGRSPYSRSPYSRSRYSRSPY |
| | SRSRYRESRYGGSHYSSSGYSNSRYSRYHSSRSHS |
| | KSGSSTSSRSASTSKSSSARRSKSSSVSRSRSRSRSS |
| | SMTRSPPRVSKRKSKSRSRSKRPPKSPEEEGQMSS |
| SNRPB-1 | TVGKSSKMLQHIDYRMRCILQDGRIFIGTFKAFDK |
| | HMNLILCDCDEFRKIKPKNSKQAEREEKRVLGLVL |
| | LRGENLVSMTVEGPPPKDTGIARVPLAGAAGGPGI |
| | GRAAGRGIPAGVPMPQAPAGLAGPVRGVGGPSQQ |
| | VMTPQGRGTVAAAAAAATASIA |
| FUBP1-3 | PPGPPGPGTPMGPYNPAPYNPGPPGPAPHGPPAPY |
| | APQGWGNAYPHWQQQAPPDPAKAGTDPNSAAW |
| | AAYYAHYYQQQAQPPPAAPAGAPTTTQTNGQGD |
| | QQNPAPAGQVDYTKAWEEYYKKMGQAVPAPTG |
| | APPGGQPDYSAAWAEYYRQQAAYYAQTSPQGMP |
| | QHPPAPQCRFDPASIELALL |
| U2AF2-2 | SDFDEFERQLNENKQERDKENRHRKRSHSRSRSRD |
| | RKRRSRSRDRRNRDQRSASRDRRRRSKPLTRGAK |
| | EEHGGLIRSPRHEKKKKVRKYWDVPPPGFEHITPM |
| | QYKAMQAAGQIPATALLPTMTPDGLAVTPTPVPV |
| | VGSQMTRQAR |
| SRSF10-2 | YRRSRSRSYERRRSRSRSFDYNYRRSYSPRNSRPTG |
| | RPRRSRSHSDNDRFKHRNRSFSRSKSNSRSRSKSQP |
| | KKEMKAKSRSRSASHTKTRGTSKTDSKTHYKSGS |
| | RYEKESRKKEPPRSKSQSRSQSRSRSKSRSRSWTSP |
| | KSSGH |

### Methods

Generation of expression plasmids for MCP and dCas13d-fused RBPs and RBP truncations. The majority of ORF clones were obtained in pENTR vectors from the CCSB human ORFcome collection (Dana-Farber Cancer Institute) or the DNASU Plasmid Repository (Arizona State University). For truncations, domain structures were determined using InterProScan on the amino acid sequences of the full-length protein and informed truncation design. Truncations and ORFs that were ordered in standard expression vectors were amplified by PCR (Phusion polymerase, NEB) with oligonucleotide primers containing attB recombination sites and recombined into pDONR211 using BP clonase II (Thermo Fisher). ORFs were then recombined into one of two custom pEF DEST51 destination vectors (Themro Fisher). For MCP-fusions, the destination vector is engineered to direct expression of the ORFs as fusion proteins with a V5 epitope tag and MCP appended C-terminally and under the control of the EF1-alpha promoter to create ORF-V5-MCP constructs. For dCas13d-fusions, the MCP is simply replaced with dCas13d for the generation of ORF-V5-dCas13d constructs. Table 2 contains sequences of both destination vectors. The identity of all cDNA clones was verified by Sanger sequencing. Plasmid libraries are available on Adgene (155390-156159). Table 3 lists all ORFs and relevant information.

Cell lines. Lenti-X HEK293T cells were purchased from Takara Bio and were not further authenticated. Cells were routinely tested for mycoplasma contamination with a MycoAlert mycoplasma test kit (Lonza) and were found negative for mycoplasma.

Generation of constructs. The lucMAPT Reporter was first constructed through a three-fragment Gibson Assembly using a homebrew enzyme mix (OpenWetWare). Fragments were generated by performing PCT on sub-fragments to generate complementary overhangs followed by annealing, amplification, and agarose gel extraction. The first fragment consists of Firefly luciferase, MAPT exon 9, and the 5′-most 500 base pairs of MAPT intron 9. The second fragment consists of the 3′-most 500 base pairs of MAPT intron 9, modified MAPT exon 10, and the 5′-most 500 base pairs of MAPT intron 10. The third fragment consists of the 3′-most 500 base pairs of MAPT intro 10, MAPT exon 11, and Renilla luciferase. The luciferase ORFs were cloned from previously generated plasmids. MAPT exons were ordered as synthetic oligonucleotides. MAPT intronic sequences were amplified from genomic DNA isolated from Lenti-X HEK293T cells. All PCR was performed using KAPA HiFi HotStart ReadyMix (Roche #7958935001).

The lucMAPT-MS2 reporters were generated with MAPT exon 10 and the flanking 100 intronic base pairs in either direction from the splice sited removed from the construct and replaced with a cloning site containing BamHI and EcoRI cut sites through PCR followed by two-fragment Gibson Assembly to generate a customizable backbone. Inserts containing MAPT exon 10, the flanking 100 base pairs, and the MS2 stem-loop sequence in the desired position were cloned into the backbone through one-fragment Gibson Assembly into pcDNA3.1 (−) mammalian expression vector (Thermo Fisher #V79520) to construct lucMAPT-MS2 reporters. Inserts containing other AS exons and flanking sequences are used to generate other reporters used. Sequences of reporters are found in Table 3.

Luciferase reporter screens. Reverse transcription was done using 96-well Solid Black Flat Bottom Polystyrene TC-treated Microplates (Corning #3916) coated with 75 μL Poly-D-lysine hydrobromide (Sigma-Aldrich #P6407-5 MG) dissolved in water at 1 g/L and further diluted in 1:5 in 1×DPBS (Corning #21-031-CV) overnight in a tissue culture incubator. Plates were rinsed twice with 1×DPBS and dried. A 1:1 mix of lucMAPT-MS2 reporter and an ORF-V5-MCP construct with a total of 100 gm DNA were added to a mixture of Lipofectamine 3000 and P3000 reagents (Thermo Fisher #L30000001) diluted in Opti-MEM Reduced Serum Media (Gibco #31985062) and incubated for 15 minutes. The mixture of DNA and transfection reagent was transferred to the PDL-coated 96-well plate. 75 μL of Lenti-X HEK293T cells were plated at a concentration of 266,666 cells/mL. The mixture was incubated for 48 hours in a standard tissue culture incubator.

For the dual-luciferase readout, luminescence was generated using the Dual-Glo Luciferase Assay System (Promega #E2980). Cells were removed from the incubator to cool to room temperature for 30 minutes. 75 μL Dual-Glo Luciferase Reagent was added directly to cells and thoroughly mixed using a Microplate Genie Plate Shaker (Scientific Industries). The reaction was briefly centrifuged and allowed to incubate at room temperature for 10 minutes. Luminescence was measured using a Spark Multimode Microplate Reader (Tecan) with a 500 ms signal interaction time at room temperature. The same process was repeated for Renilla luciferase luminescence using the Dual-Glo Stop & Glo reagent.

TABLE 3
Target RNA Molecules for High-Throughput Assay.

| Name | Sequence |
|---|---|
| lucMAPT- | atgggcagcggtaagcctatccctaaccctctcctcggtctcgattctacgggcagcatggccgatgctaagaa |
| 30D | cattaagaagggccctgctcccttctaccctctggaggatggcaccgctggcgagcagctgcacaaggccat |
| | gaagaggtatgccctggtgcctggcaccattgccttcaccgatgcccacattgaggtggacatcacctatgccg |
| | agtacttcgagatgtctgtgcgcctggccgaggccatgaagaggtacggcctgaacaccaaccaccgcatcg |
| | tggtgtgctctgagaactctctgcagttcttcatgccagtgctgggcgccctgttcatcggagtggccgtggccc |
| | ctgctaacgacatttacaacgagcgcgagctgctgaacagcatgggcatttctcagcctaccgtggtgttcgtgt |
| | ctaagaagggcctgcagaagatcctgaacgtgcagaagaagctgcctatcatccagaagatcatcatcatgga |
| | ctctaagaccgactaccagggcttccagagcatgtacacattcgtgacatctcatctgcctcctggcttcaacga |
| | gtacgacttcgtgccagagtctttcgacagggacaaaaccattgccctgatcatgaacagctctgggtctaccg |
| | gcctgcctaagggcgtggccctgcctcatcgcaccgcctgtgtgcgcttctctcacgcccgcgaccctattttc |
| | ggcaaccagatcatccccgacaccgctattctgagcgtggtgccattccaccacggcttcggcatgttcaccac |
| | cctgggctacctgatttgcggctttcgggtggtgctgatgtaccgcttcgaggaggagctgttcctgcgcagcct |
| | gcaagactacaaaattcagtctgccctgctggtgccaaccctgttcagcttctccgctaagagcaccctgatcga |
| | caagtacgacctgtctaacctgcacgagattgcctctggcggcgccccactgtctaaggaggtgggcgaagc |
| | cgtggccaagcgctttcatctgccaggcatccgccagggctacggcctgaccgagacaaccagcgccattct |
| | gattaccccagagggcgacgacaagcctggcgccgtgggcaaggtggtgccattcttcgaggccaaggtgg |
| | tggacctggacaccggcaagaccctgggagtgaaccagcgcggcgagctgtgtgtgcgcggccctatgatt |
| | atgtccggctacgtgaataaccctgaggccacaaacgccctgatcgacaaggacggctggctgcactctggc |
| | gacattgcctactgggacgaggacgagcacttcttcatcgtggaccgcctgaagtctctgatcaagtacaagg |
| | gctaccaggtggccccagccgagctggagtctatcctgctgcagcaccctaacattttcgacgccggagtggc |
| | cggcctgcccgacgacgatgccggcgagctgcctgccgccgtcgtcgtgctggaacacggcaagaccatg |
| | accgagaaggagatcgtggactatgtggccagccaggtgacaaccgccaagaagctgcgcggcggagtgg |
| | tgttcgtggacgaggtgcccaagggcctgaccggcaagctggacgcccgcaagatccgcgagatcctgatc |
| | aaggctaagaaaggcggcaagatcgccgtgagtgaacctccaaaatcaggggatcgcagcggctacagca |
| | gccccggctccccaggcactcccagcagccgctcccgcaccccgtcccttccaaccccacccacccgggag |
| | cccaagaaggtggcagtggtccgtactccacccaagtcgccgtcttccgccaagagccgcctgcagacagc |
| | ccccgtgcccatgccagacctgaagaatgtcaagtccaagatcggctccactgagaacctgaagcaccagcc |
| | gggaggcgggaaggtgagagtggctggctgcgcgtggaggtgtggggggctgcgcctggaggggtaggg |
| | ctgtgcctggaagggtagggctgcgcctggaggtgcgcggttgagcgtggagtcgtgggactgtgcatgga |
| | ggtgtggggctccccgcacctgagcacccccgcataacaccccagtcccctctggaccctcttcaaggaagtt |
| | cagttctttattgggctctccactacactgtgagtgccctcctcaggcgagagaacgttctggctcttctcttgccc |
| | cttcagcccctgttaatcggacagagatggcagggctgtgtctccacggccggaggctctcatagtcagggca |
| | cccacagtggttccccacctgccttctgggcagaatacactgccacccataggtcagcatctccactcgtgggc |
| | catctgcttaggttgggttcctctggattctggggagattgggggttctgttttgatcagctgattcttctgggagaa |
| | atccacaggtgattctgatgcccggcaggcttgagaacagccgcagggagttctctgggaatgtgccggtgg |
| | gtctagccaggtgtgagtggagatgccggggaacttcctattactaactcgtcagtgtggccgaacacatttttc |
| | acttgacctcaggctggtgaacgctcccctctggggttcaggcctcacgatgccatccttttgtgaagtgaggac |
| | ctgcaatcccagcttcgtaaagcccgctggaaatcactcacacttctgggatgccttcagagcagccctctatcc |
| | cttcagctcccctgggatgtgactcaacctcccgtcactccccagactacctctgccaagtccgaaagtggagg |
| | catccttgcgagcaagtaggcgggtccagggtggtgcatgtcactcatcgaaagtggaggcgtccttgcgag |
| | caagcaggcgggtccagggtggcgtgtcactcatccttttttctggctaccaaaggtgcagataattaataagaa |
| | gctggatcttagcaacgtccagtccaagtgtggctcaaaggataatatctaacacgtcccgggaggcggcagt |
| | gtgagtaccttcacacgtcccatgcgccgtacatgaggatcacccatgtgctgtggcttgaattattaggaagtg |
| | gtgtgagtgcgtacacttgcgagacactgcatagaataaatccttcttgggctctcaggatctggctgcgacctc |
| | tgggtgaatgtagcccggctccccacattcccccacacggtccactgttcccagaagccccttcctcatattcta |
| | ggagggggtgtcccagcatttctgggtcccccagcctgcgcaggctgtgtggacagaatagggcagatgac |
| | ggaccctctctccggaccctgcctgggaagctgagaatacccatcaaagtctccttccactcatgcccagccct |
| | gtccccaggagccccatagcccattggaagttgggctgaaggtggtggcacctgagactgggctgccgcctc |
| | ctcccccgacacctgggcaggttgacgttgagtggctccactgtggacaggtgacccgtttgttctgatgagcg |
| | gacgcctcccctctttgaggcccagcagataccccactcctgcctttccagcaagatttttcagatgctgtgcata |
| | ctcatcatattgatcacttttttcttcatgcctgattgtgatctgtcaatttcatgtcaggaaagggagtgacatttttac |
| | acttaagcgtttgctgagcaaatgtctgggtcttgcacaatgacaatgggtccctgtttttcccagaggctcttttgt |
| | tctgcagggattgaagacactccagtcccacagtccccagctcccctggggcagggttggcagaatttcgaca |
| | acacatttttccaccctgactaggatgtgctcctcatggcagctgggaaccactgtccaataagggcctgggctt |
| | acacagctgcttctcattgagttacacccttaataaaataatcccattttatcctttttgtctctctgtcttcctctctctc |
| | tgcctttcctcttctctctcctcctctctcatctccaggtgcaaatagtctacaaaccagttgacctgagcaaggtga |
| | cctccaagtgtggctcattaggcaacatccatcataaaccagcttccaaggtgtatgaccccgagcaacgcaaa |
| | cgcatgatcactgggcctcagtggtgggctcgctgcaagcaaatgaacgtgctggactccttcatcaactacta |
| | tgattccgagaagcacgccgagaacgccgtgatttttctgcatggtaacgctgcctccagctacctgtggaggc |
| | acgtcgtgcctcacatcgagcccgtggctagatgcatcatccctgatctgatcggaatgggtaagtccggcaa |
| | gagcgggaatggctcatatcgcctcctggatcactacaagtacctcaccgcttggttcgagctgctgaaccttcc |
| | aaagaaaatcatctttgtgggccacgactggggggcttgtctggcctttcactactcctacgagcaccaagaca |
| | agatcaaggccatcgtccatgctgagagtgtcgtggacgtgatcgagtcctgggacgagtggcctgacatcga |
| | ggaggatatcgccctgatcaagagcgaagagggcgagaaaatggtgcttgagaataacttcttcgtcgagac |
| | catgctcccaagcaagatcatgcggaaactggagcctgaggagttcgctgcctacctggagccattcaagga |
| | gaagggcgaggttagacggcctaccctctcctggcctcgcgagatccctctcgttaagggaggcaagcccga |
| | cgtcgtccagattgtccgcaactacaacgcctaccttcgggccagcgacgatctgcctaagatgttcatcgagt |
| | ccgaccctgggttcttttccaacgctattgtcgagggagctaagaagttccctaacaccgagttcgtgaaggtga |
| | agggcctccacttcagccaggaggacgctccagatgaaatgggtaagtacatcaagagcttcgtggagcgcg |
| | tgctgaagaacgagcag |
| lucMAPT- | atgggcagcggtaagcctatccctaaccctctcctcggtctcgattctacgggcagcatggccgatgctaagaa |
| 30U | cattaagaagggccctgctcccttctaccctctggaggatggcaccgctggcgagcagctgcacaaggccat |
| | gaagaggtatgccctggtgcctggcaccattgccttcaccgatgcccacattgaggtggacatcacctatgccg |
| | agtacttcgagatgtctgtgcgcctggccgaggccatgaagaggtacggcctgaacaccaaccaccgcatcg |
| | tggtgtgctctgagaactctctgcagttcttcatgccagtgctgggcgccctgttcatcggagtggccgtggccc |
| | ctgctaacgacatttacaacgagcgcgagctgctgaacagcatgggcatttctcagcctaccgtggtgttcgtgt |
| | ctaagaagggcctgcagaagatcctgaacgtgcagaagaagctgcctatcatccagaagatcatcatcatgga |
| | ctctaagaccgactaccagggcttccagagcatgtacacattcgtgacatctcatctgcctcctggcttcaacga |
| | gtacgacttcgtgccagagtctttcgacagggacaaaaccattgccctgatcatgaacagctctgggtctaccg |
| | gcctgcctaagggcgtggccctgcctcatcgcaccgcctgtgtgcgcttctctcacgcccgcgaccctattttc |
| | ggcaaccagatcatccccgacaccgctattctgagcgtggtgccattccaccacggcttcggcatgttcaccac |
| | cctgggctacctgatttgcggctttcgggtggtgctgatgtaccgcttcgaggaggagctgttcctgcgcagcct |
| | gcaagactacaaaattcagtctgccctgctggtgccaaccctgttcagcttctccgctaagagcaccctgatcga |
| | caagtacgacctgtctaacctgcacgagattgcctctggcggcgccccactgtctaaggagggggcgaagc |
| | cgtggccaagcgctttcatctgccaggcatccgccagggctacggcctgaccgagacaaccagcgccattct |
| | gattaccccagagggcgacgacaagcctggcgccgtgggcaaggtggtgccattcttcgaggccaaggtgg |
| | tggacctggacaccggcaagaccctgggagtgaaccagcgcggcgagctgtgtgtgcgcggccctatgatt |
| | atgtccggctacgtgaataaccctgaggccacaaacgccctgatcgacaaggacggctggctgcactctggc |
| | gacattgcctactgggacgaggacgagcacttcttcatcgtggaccgcctgaagtctctgatcaagtacaagg |
| | gctaccaggtggccccagccgagctggagtctatcctgctgcagcaccctaacattttcgacgccggagtggc |
| | cggcctgcccgacgacgatgccggcgagctgcctgccgccgtcgtcgtgctggaacacggcaagaccatg |
| | accgagaaggagatcgtggactatgtggccagccaggtgacaaccgccaagaagctgcgcggcggagtgg |
| | tgttcgtggacgaggtgcccaagggcctgaccggcaagctggacgcccgcaagatccgcgagatcctgatc |
| | aaggctaagaaaggcggcaagatcgccgtgagtgaacctccaaaatcaggggatcgcagcggctacagca |
| | gccccggctccccaggcactcccagcagccgctcccgcaccccgtcccttccaaccccacccacccgggag |
| | cccaagaaggtggcagtggtccgtactccacccaagtcgccgtcttccgccaagagccgcctgcagacagc |
| | ccccgtgcccatgccagacctgaagaatgtcaagtccaagatcggctccactgagaacctgaagcaccagcc |
| | gggaggcgggaaggtgagagtggctggctgcgcgtggaggtgtggggggctgcgcctggaggggtaggg |
| | ctgtgcctggaagggtagggctgcgcctggaggtgcgcggttgagcgtggagtcgtgggactgtgcatgga |
| | ggtgtggggctccccgcacctgagcacccccgcataacaccccagtcccctctggaccctcttcaaggaagtt |
| | cagttctttattgggctctccactacactgtgagtgccctcctcaggcgagagaacgttctggctcttctcttgccc |
| | cttcagcccctgttaatcggacagagatggcagggctgtgtctccacggccggaggctctcatagtcagggca |
| | cccacagtggttccccacctgccttctgggcagaatacactgccacccataggtcagcatctccactcgtgggc |
| | catctgcttaggttgggttcctctggattctggggagattgggggttctgttttgatcagctgattcttctgggagaa |
| | atccacaggtgattctgatgcccggcaggcttgagaacagccgcagggagttctctgggaatgtgccggtgg |
| | gtctagccaggtgtgagtggagatgccggggaacttcctattactaactcgtcagtgtggccgaacacatttttc |
| | acttgacctcaggctggtgaacgctcccctctggggttcaggcctcacgatgccatccttttgtgaagtgaggac |
| | ctgcaatcccagcttcgtaaagcccgctggaaatcactcacacttctgggatgccttcagagcagccctctatcc |
| | cttcagctcccctgggatgtgactcaacctcccgtcactccccagactacctctgccaagtccgaaagtggagg |
| | catccttgcgagcaagtagggggcgcatgtcactcatcgaaagtggaggcgtccttgcgagcaagcaggcg |
| | ggtccagggtggcgtacatgaggatcacccatgtgtcactcatccttttttctggctaccaaaggtgcagataatt |
| | aataagaagctggatcttagcaacgtccagtccaagtgtggctcaaaggataatatctaacacgtcccgggag |
| | gcggcagtgtgagtaccttcacacgtcccatgcgccgtgctgtggcttgaattattaggaagtggtgtgagtgc |
| | gtacacttgcgagacactgcatagaataaatccccaggatctggctgcgacctctgggtgaatgtagcccggct |
| | ccccacattcccccacacggtccactgttcccagaagccccttcctcatattctaggagggggtgtcccagcatt |
| | tctgggtcccccagcctgcgcaggctgtgtggacagaatagggcagatgacggaccctctctccggaccctg |
| | cctgggaagctgagaatacccatcaaagtctccttccactcatgcccagccctgtccccaggagccccatagc |
| | ccattggaagttgggctgaaggtggtggcacctgagactgggctgccgcctcctcccccgacacctgggcag |
| | gttgacgttgagtggctccactgtggacaggtgacccgtttgttctgatgagcggacgcctcccctctttgaggc |
| | ccagcagataccccactcctgcctttccagcaagatttttcagatgctgtgcatactcatcatattgatcacttttttc |
| | ttcatgcctgattgtgatctgtcaatttcatgtcaggaaagggagtgacatttttacacttaagcgtttgctgagcaa |
| | atgtctgggtcttgcacaatgacaatgggtccctgtttttcccagaggctcttttgttctgcagggattgaagacac |
| | tccagtcccacagtccccagctcccctggggcagggttggcagaatttcgacaacacatttttccaccctgacta |
| | ggatgtgctcctcatggcagctgggaaccactgtccaataagggcctgggcttacacagctgcttctcattgagt |
| | tacacccttaataaaataatcccattttatcctttttgtctctctgtcttcctctctctctgcctttcctcttctctctcctcc |
| | tctctcatctccaggtgcaaatagtctacaaaccagttgacctgagcaaggtgacctccaagtgtggctcattag |
| | gcaacatccatcataaaccagcttccaaggtgtatgaccccgagcaacgcaaacgcatgatcactgggcctca |
| | gtggtgggctcgctgcaagcaaatgaacgtgctggactccttcatcaactactatgattccgagaagcacgccg |
| | agaacgccgtgatttttctgcatggtaacgctgcctccagctacctgtggaggcacgtcgtgcctcacatcgag |
| | cccgtggctagatgcatcatccctgatctgatcggaatgggtaagtccggcaagagcgggaatggctcatatc |
| | gcctcctggatcactacaagtacctcaccgcttggttcgagctgctgaaccttccaaagaaaatcatctttgtggg |
| | ccacgactggggggcttgtctggcctttcactactcctacgagcaccaagacaagatcaaggccatcgtccat |
| | gctgagagtgtcgtggacgtgatcgagtcctgggacgagtggcctgacatcgaggaggatatcgccctgatc |
| | aagagcgaagagggcgagaaaatggtgcttgagaataacttcttcgtcgagaccatgctcccaagcaagatc |
| | atgcggaaactggagcctgaggagttcgctgcctacctggagccattcaaggagaagggcgaggttagacg |
| | gcctaccctctcctggcctcgcgagatccctctcgttaagggaggcaagcccgacgtcgtccagattgtccgc |
| | aactacaacgcctaccttcgggccagcgacgatctgcctaagatgttcatcgagtccgaccctgggttcttttcc |
| | aacgctattgtcgagggagctaagaagttccctaacaccgagttcgtgaaggtgaagggcctccacttcagcc |
| | aggaggacgctccagatgaaatgggtaagtacatcaagagcttcgtggagcgcgtgctgaagaacgagcag |
| HNRNPD | gtcggccattttaggtggtccgcggcggcgccattaaagcgaggaggaggcgagagcggccgccgctggt |
| isoform a | gcttattcttttttagtgcagcgggagagagcgggagtgtgcgccgcgcgagagtgggaggcgaagggggc |
| | aggccagggagaggcgcaggagcctttgcagccacgcgcgcgccttccctgtcttgtgtgcttcgcgaggta |
| | gagcgggcgcgcggcagcggcggggattactttgctgctagtttcggttcgcggcagcggcgggtgtagtct |
| | cggcggcagcggcggagacactagcactatgtcggaggagcagttcggcggggacggggcggcggcag |
| | cggcaacggcggcggtaggcggctcggcgggcgagcaggagggagccatggtggcggcgacacaggg |
| | ggcagcggcggcggcgggaagcggagccgggaccgggggcggaaccgcgtctggaggcaccgaagg |
| | gggcagcgccgagtcggagggggcgaagattgacgccagtaagaacgaggaggatgaaggccattcaaa |
| | ctcctccccacgacactctgaagcagcgacggcacagcgggaagaatggaaaatgtttataggaggccttag |
| | ctgggacactacaaagaaagatctgaaggactacttttccaaatttggtgaagttgtagactgcactctgaagtta |
| | gatcctatcacagggcgatcaaggggttttggctttgtgctatttaaagaatcggagagtgtagataaggtcatg |
| | gatcaaaaagaacataaattgaatgggaaggtgattgatcctaaaagggccaaagccatgaaaacaaaagag |
| | ccggttaaaaaaatttttgttggtggcctttctccagatacacctgaagagaaaataagggagtactttggtggttt |
| | tggtgaggtggaatccatagagctccccatggacaacaagaccaataagaggcgtgggttctgctttattacctt |
| | taaggaagaagaaccagtgaagaagataatggaaaagaaataccacaatgttggtcttagtaaatgtgaaataa |
| | aagtagccatgtcgaaggaacaatatcagcaacagcaacagtggggatctagaggaggatttgcaggaaga |
| | gctcgtggaagaggtggtggccccagtcaaaactggaaccagggatatagtaactattggaatcaaggctatg |
| | gcaactatggatataacagccaaggttacggtggttatggaggatatgactacactggttacaacaactactatg |
| | gatatggtgattatagcaaccagcagagtggttatgggaaggtatccaggcgaggtggtcatcaaaatagctac |
| | aaaccatactaaattattccatttgcaacttatccccaacaggtggtgaagcagtattttccaatttgaagattcattt |
| | gaaggtggctcctgccacctgctaatagcagttcaaactaaattttttgtatcaagtccctgaatggaagtatgac |
| | gttgggtccctctgaagtttaattctgagttctcattaaaagaaatttgctttcattgttttatttcttaattgctatgcttc |
| | agaatcaatttgtgttttatgccctttcccccagtattgtagagcaagtcttgtgttaaaagcccagtgtgacagtgt |
| | catgatgtagtagtgtcttactggttttttaataaatccttttgtataaaaatgtattggctcttttatcatcagaatagg |
| | aaaaattgtcatggattcaagttattaaaagcataagtttggaagacaggcttgccgaaattgaggacatgattaa |
| | aattgcagtgaagtttgaaatgtttttagcaaaatctaatttttgccataatgtgtcctccctgtccaaattgggaatg |
| | acttaatgtcaatttgtttgttggttgttttaataatacttccttatgtagccattaagatttatatgaatattttcccaaat |
| | gcccagtttttgcttaatatgtattgtgctttttagaacaaatctggataaatgtgcaaaagtacccctttgcacagat |
| | agttaatgttttatgcttccattaaataaaaaggacttaaaatctgttaattataatagaaatgcggctagttcagaga |
| | gatttttagagctgtggtggacttcatagatgaattcaagtgttgagggaggattaaagaaatatataccgtgtttat |
| | gtgtgtgtgcttatttgtttgaatgattttattttccatttctcaaaggttttatttttttggttagggccttaaaatttcagg |
| | actgtgattattagtatgtgtgcctaaggaactttttgagtcactcttaagaaagtgaaactgaagagtctaagtgat |
| | aactataggattaagtcagaattgtttttcctgtcatttgttggaagcttcttgagttctgttattagcattcagggaatt |
| | gatacccatcaacttgaatggaaaatcgtttgtaggtattacttaagtgaatgttaagagttccaccctgagtggta |
| | atctaaggctgtgcagtcagttacttcagactgctcagaatagttcattagaaaggtaacaaatgagaaatgtatt |
| | attatacagttctatagtagtgaagtgatggaatacctttcttacttttgtggagttacatctgatgctaagaatttgac |
| | ctccaactaagcaaacattttaatgagcaaaagttagtgttattaaagtttttttatgatagatccaaattgaggacct |
| | gtgtcctgtttttataagattgcaacccagctatgctcatttgtttatgttttgtatatggctgcttttgtgttacagtggt |
| | agagtttagtagttaggacagagacctgcaaagcaaaataatttacagtctggccctttacagaaaagtttgctg |
| | actcatggtcaaaataaatgaaaattttttgtgttagggttgttaagctagggttctttttggtatcatatgcttattttat |
| | gtaaatctctcaataaaaaattatttttaagaga |

TABLE 4
Target RNA Sequences in Target RNA Molecules for
High-Throughput Assay.

| Name | Sequence |
|---|---|
| lucMAPT Protospacer 1 | CCGTGCTGTGGCTTGAATTATTA |
| lucMAPT Protospacer 2 | GTGCTGTGGCTTGAATTATTAGG |
| lucMAPT Protospacer 3 | GCTGTGGCTTGAATTATTAGGAA |
| lucMAPT Protospacer 4 | GTGGCGTGTCACTCATCCTTTTT |
| lucMAPT Protospacer 5 | GGGTGGCGTGTCACTCATCCTTT |
| lucMAPT Protospacer 6 | GGTGGCGTGTCACTCATCCTTTT |
| HNRNPD Exon 7 | TGGATAGGCAGAAAGGTTAGTGT |
| Protospacer 1 | |
| HNRNPD Exon 7 | GCTATTTGACATTTATTTTGTAC |
| Protospacer 2 | |
| HNRNPD Exon 7 | GTAAGTACTATACTTTTTATATT |
| Protospacer 3 | |
| HNRNPD Exon 7 | TAGTGAACCTATTAATGTGCTGC |
| Protospacer 4 | |
| HNRNPD Exon 7 | TGCTGATCTTCTGACTTTAGTGA |
| Protospacer 5 | |
| HNRNPD Exon 7 | TCCTGAAGTAAAGATCTTTGCTG |
| Protospacer 6 | |

For the statistical analysis, relative ψ values were calculated as described using the pandas library in Python v3.10.11. All plots generated from Python were generated using JupyterLab 4.04. Significance between candidate and negative control conditions was assessed by calculating p-value through a one-tailed independent t-test using the ttest_ind function in scipy.

Modulation of splicing with dCas13d fusions. Transfection was performed as described for the luciferase reporter screens. The plasmid DNA transfected consisted with 10 ng lucMAPT Reporter DNA, 45 ng gRNA plasmid, and 45 ng dCas13d-RBP fusion. Dual-luciferase readout was collected as described for the luciferase reporter screens. gRNA sequences were designed using the cas13design tool. Transfection for modulation of endogenous targets was performed in 24-well plates with 250 ng gRNA plasmid DNA and 250 ng dCas13d-RBP fusion.

### Experiment No. 2

Experiment No. 2 is a more detailed report of the materials and methods described in Experiment No. 1. Full bibliographic citations for the references identified in Experiment No. 2 by an Arabic number are provided below, immediately preceding the claims.

### Development of Tethered-Function Splicing Reporter Assays

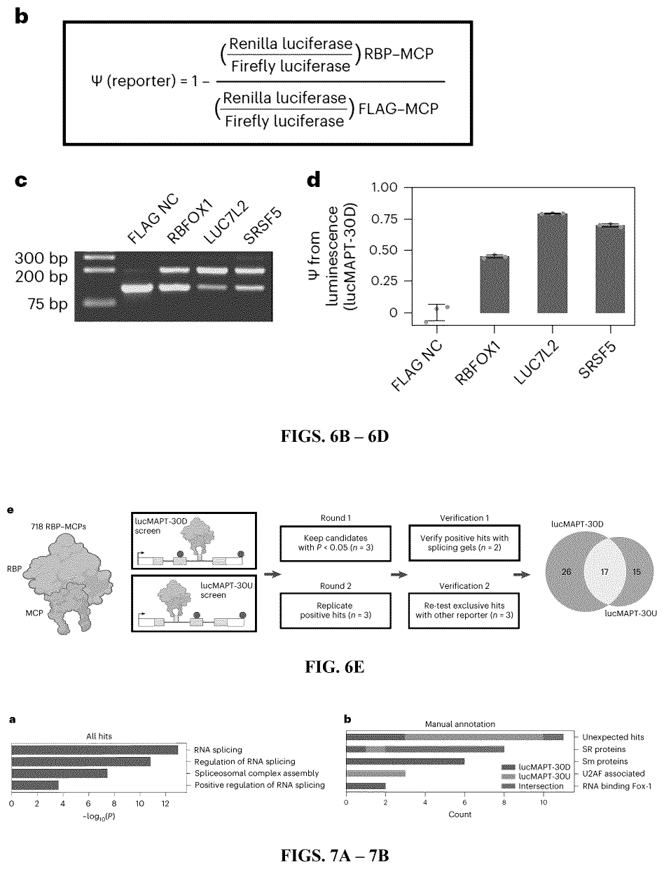

Applicant constructed two dual-luciferase tethered AS minigene reporter systems based on the splicing event of MAPT (Microtubule Associated Protein Tau) exon 10 (FIG. 6A, and FIG. 12A, Large-scale evaluation of the ability of RNA-binding proteins to activate exon inclusion. Nat Biotechnol. September; 42 (9): 1429-1441. doi: 10.1038/s41587-023-02014-0. Epub 2024 Jan. 2. Erratum in: Nat Biotechnol. 2024 September; 42 (9): 1467. doi: 10.1038/s41587-024-02178-3. PMID: 38168984; PMCID: PMC11389820, hereinafter, Schmok J C (2024), incorporated herein by reference)²¹ which is predominantly excluded from the mature mRNA in HEK293T cells. The first reporter contains the MS2 hairpin 30 base pairs downstream of the 5′ splice site (lucMAPT-30D), and the second contains the MS2 hairpin 30 base pairs upstream of the 3′ splice site (lucMAPT-30U). The MS2 hairpin recruits MS2 coat protein (MCP) fused to RBP open reading frames (ORFs) to determine the effect on AS of the exon when RBPs are tethered to various positions on the RNA.

Both minigenes are flanked by a constitutively included Firefly luciferase ORF at the 5′ end and a conditionally included Renilla luciferase ORF at the 3′ end to permit inference of exon inclusion. Firefly luciferase is expressed independent of exon skipping, but inclusion of the tau exon harboring a stop codon terminates translation upstream of Renilla luciferase. Applicant used changes in luminescence in experimental conditions to determine changes in the percent-spliced-in (ψ) of the AS exon when compared to a negative control (FIG. 6B). The AS exon is the penultimate exon, so Applicant inserted the stop codon within 50 base pairs of the 5′ splice site in order to minimize sensitivity of the long isoform to nonsense-mediated decay (NMD)²².

To validate Applicant's assay, Applicant co-transfected the lucMAPT-30D reporter with fusion proteins composed of known regulators of exon inclusion and MCP. For a negative control (NC), Applicant used a construct containing an array of 3 FLAG epitope tags fused to MCP (FLAG NC). Applicant compared ψ value as measured by the reporter readout to an RNA-level validation (FIG. 6C, FIG. 6D). Compared to FLAG NC, MCP-fused proteins LUC7L2, SRSF5, and RBFOX1 increased exon inclusion as measured by both techniques in decreasing order of intensity. To verify that effector recruitment was mediated by the MS2-MCP system, Applicant co-transfected lucMAPT-30D with an RBFOX1 plasmid lacking the MCP fusion. This did not activate the reporter (FIG. 12B), incorporated herein by reference). As Applicant designed these reporters to minimize sensitivity to NMD, Applicant tested the response of the reporters to NMD perturbation by testing the reporter readout in response to shRNA-mediated knockdown of UPF1, the central effector of NMD²³, and SMG7, a non-essential NMD factor²⁴ (FIGS. 12C-12E). Applicant detected a minor (<10%) increase in long isoform abundance following NMD perturbation, indicating that the early stop codon-containing long isoform is to some degree sensitive to NMD. For the purposes of Applicant's studies, where the NMD environment is consistent and candidates are recruited specifically to pre-mRNA by MS2-containing introns, Applicant deemed it acceptable. Based on these validations, Applicant moved forward with these reporters to screen Applicant's RBP-MCP library.

Tethering Assays Identify RBPs that Induce Exon Inclusion

Applicant evaluated 718 RBP ORFs fused to MCP for their ability to induce exon inclusion (Supplementary Table 1 in Schmok J C (2024), incorporated herein by reference). Applicant's lab previously developed the RBP-MCP library from subcloning of putative RBP ORFs16. Applicant performed two arrayed co-transfection screens with candidate RBPs in HEK293T cells, one with lucMAPT-30D, and one with lucMAPT-30U (FIG. 6E, left). Applicant analyzed all ORFs in triplicate and compared to negative (FLAG NC) and positive controls (RBFOX1-MCP for lucMAPT-30D and SRSF5-MCP for lucMAPT-30U) on the same plate (FIG. 12F). Since Applicant's analysis focused on v increases exclusively, Applicant measured statistical significance when compared to the negative control by one-tailed independent two-sample t-test.

Applicant moved forward with candidates that increased ψ significantly (p<0.05; Supplementary Table 2-3 in Schmok J C (2024), incorporated herein by reference) and verified them with further rounds of screening (FIG. 6E, middle). First, Applicant replicated the reporter results of all selected candidates and moved forward with those that again increased y significantly (p<0.05; Supplementary Table 4-5 in Schmok J C (2024), incorporated herein by reference). Applicant then verified that all positive hits induced exon inclusion of the reporter at the RNA level through agarose gel electrophoresis of amplified cDNA following the same transfection conditions (FIG. 12G, Supplementary Table 6-7 in Schmok J C (2024), incorporated herein by reference). ψ was estimated by calculating the intensity ratio of the inclusion band to the skipping band in duplicate and comparing against control conditions distributed throughout the gel. Applicant calculated p-value by one-tailed independent two-sample t-test, and hits with Bonferroni corrected p<0.05 were kept. Finally, remaining hits that exclusively activated one of the two reporters were evaluated one more time with the opposite reporter in case they were missed by the initial screen (Supplementary Table 8-9 in Schmok J C (2024), incorporated herein by reference). Following these rounds of screening, 26 hits were detected that exclusively activated lucMAPT-30D, 15 hits were detected that exclusively activated lucMAPT-30U, and 17 hits were detected that activated both reporters (Supplementary Table 10, FIG. 6E), right in Schmok J C (2024), incorporated herein by reference).

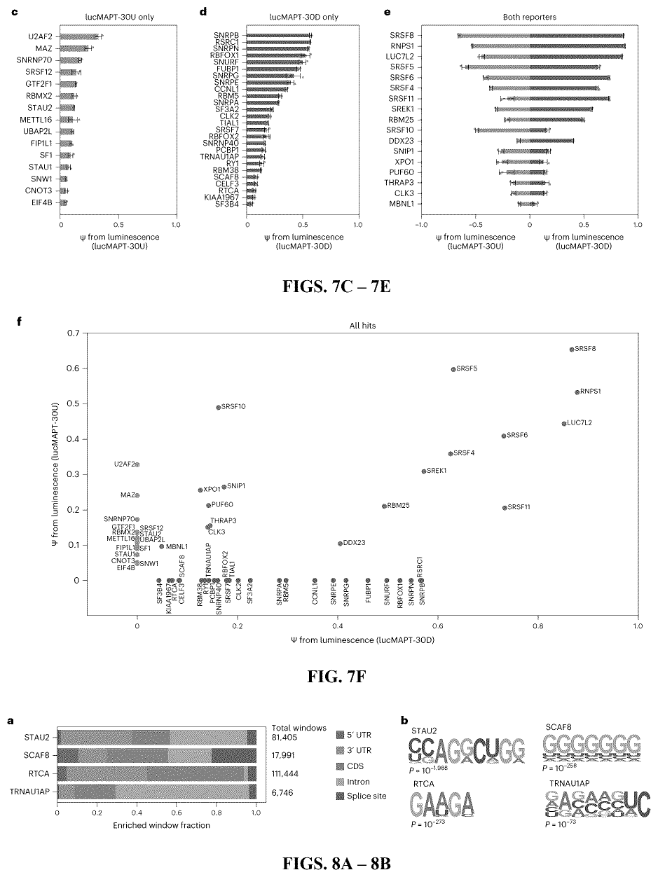

Applicant investigated the biology underlying the candidates detected from Applicant's screens. To verify that Applicant's assays robustly captured known regulators of AS, Applicant performed GO analysis on the full list of final hits. When compared to a background of the complete tethering library, GO analysis showed strong enrichment of RNA splicing-associated terms (FIG. 7A). As alternative splicing occurs in the nucleus, Applicant investigated the subcellular localization of the candidates. Applicant referenced the COMPARTMENTS subcellular localization database, which integrates evidence from text mining, high-throughput screens, literature, and prediction methods and extracted the nuclear localization confidence score for each candidate²⁵. All candidates, save two, have a nuclear confidence score of 4/5 or greater (Supplementary Table 10 in Schmok J C (2024), incorporated herein by reference). The two candidates that scored lower than 4/5 were STAU1 and EIF4B. STAU1, which scored 2.68/5, has previously been linked to splicing regulation^{26,27}. EIF4B, which scored 3.82/5, initiates translation in the cytoplasm by binding RNA substrates and recruiting ribosomes. Applicant hypothesized that this mechanism could drive a false positive when artificially driven to nuclear pre-mRNA in Applicant's tethering system, as the mechanism of spliceosome recruitment is similar. Nevertheless, a potentially nuclear role of EIF4B in splicing regulation merits future investigation. Altogether, the candidates determined by Applicant's screen are enriched for known regulators of mRNA splicing and are largely localized to the nucleus.

Applicant also detected differences in the types of RBPs identified by each screen (FIG. 7B). Both RBFOX1 and RBFOX2 exclusively activated the reporter when tethered downstream (lucMAPT-30D), consistent with the known effect of these proteins primarily causing exon inclusion when bound downstream of alternatively spliced exons^{11,28}. Three proteins associated with 3′ splice site recognition exclusively activated the upstream tethering reporter (lucMAPT-30U): U2AF2 (the large subunit of the U2 auxiliary factor), SF1, and SNW129,30. The RBPs tested from the Sm family (SNRPB, SNRPN, SNURF, SNRPG, SNRPE, SNRPA) exclusively and potently activated the downstream tethering reporter, despite the Sm ring being found in spliceosomal subunits that form at either end of the splicing junction³¹. The SR family of splicing factors was primarily represented at the intersection of both screens (SRSF8, SRSF5, SRSF6, SRSF4, SRSF11, SRSF10), however SRSF7 exclusively activated the downstream tethering reporter and SRSF12 exclusively activated the upstream tethering reporter.

As Applicant were especially interested in candidates that have not previously been associated with alternative splicing regulation, Applicant first determined candidates that were not annotated with splicing associated GO terms and have not been specifically referenced in the literature as potential splicing factors and deemed them ‘unexpected hits’. Most unexpected hits exclusively activated the upstream tethering reporter (UBAP2L, STAU2, EIF4B, CNOT3, MAZ, GTF2F1, FIP1L1), which was uncommon for known splice modulatory factors. Applicant detected three unexpected hits as exclusive activators of the downstream tethering reporter (TRNAU1AP, SCAF8, RTCA) and one as an activator of both reporters (XPO1). Next, Applicant searched for the unexpected hits on the spliceosome database (SpliccosomeDB) to determine if previous proteomics efforts have identified them as interactors with components of the spliceosome in humans³². This search yielded such evidence for SCAF8, CNOT3 and FIP1L1. SCAF8 has been detected in a supraspliceosome complex in-vivo assembled from HeLa cell extract³³ and following immunoprecipitation of CDC5L in Hela cells³⁴. CNOT3 has been detected following immunoprecipitation of SRRM1 in HeLa extract³⁵. FIP1L1 has been detected following isolation of mixed spliceosome complexes assembled in vitro from the extracts of WERI-1 retinoblastoma cells³⁶ and HeLa cells³⁷. Finally, Applicant also noted that XPO1 has a known, albeit indirect, role in mRNA splicing. XPO1 is a nuclear export receptor that shuttles the immature small nuclear RNAs of the spliceosome to the cytoplasm for maturation³⁸. Despite the preliminary evidence linking a subset of the unexpected hits to mRNA splicing, the landscape of splicing events regulated by any of the unexpected hits has not currently been characterized in any biological system.

Applicant binned hits into categories depending on whether they activated the downstream reporter only, the upstream reporter only, or activated both reporters. Binned RBPs display effect size patterns associated with their categories (FIGS. 7C-7F). For the RBPs that activated both reporters, ψ for the two reporters is correlated. A population exists among the RBPs that activated both reporters with high strength which includes the strongest overall hit, SRSF8. SRSF8 activated the highest ψ with the upstream tethering reporter and the second highest ψ for the downstream tethering reporter behind RNPS1. The downstream-only hits generally exhibited stronger activation than upstream-only hits. These categories of hits display trends in effect size, however the variance within each category highlights the diversity of mechanisms by which RBPs influence AS by proximity.

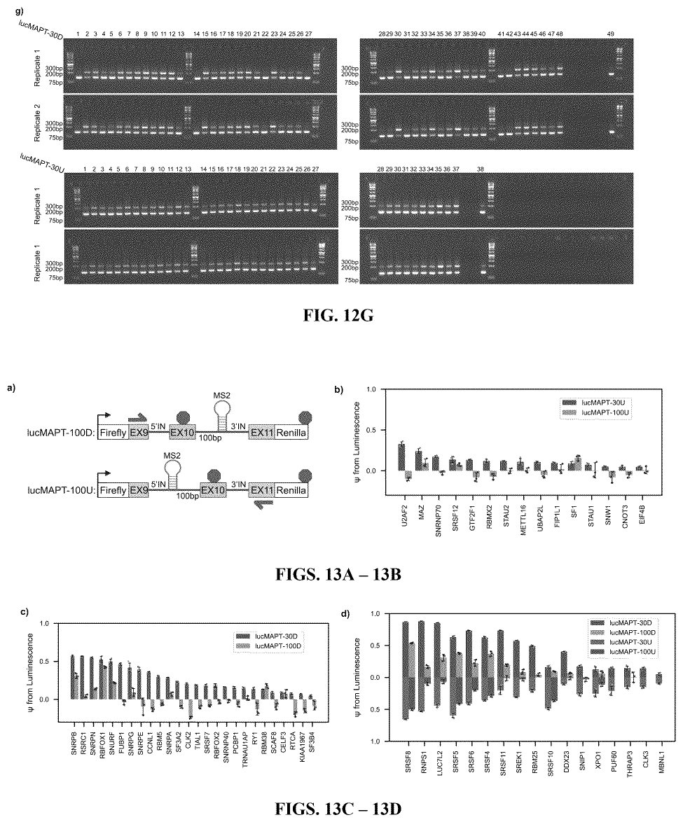

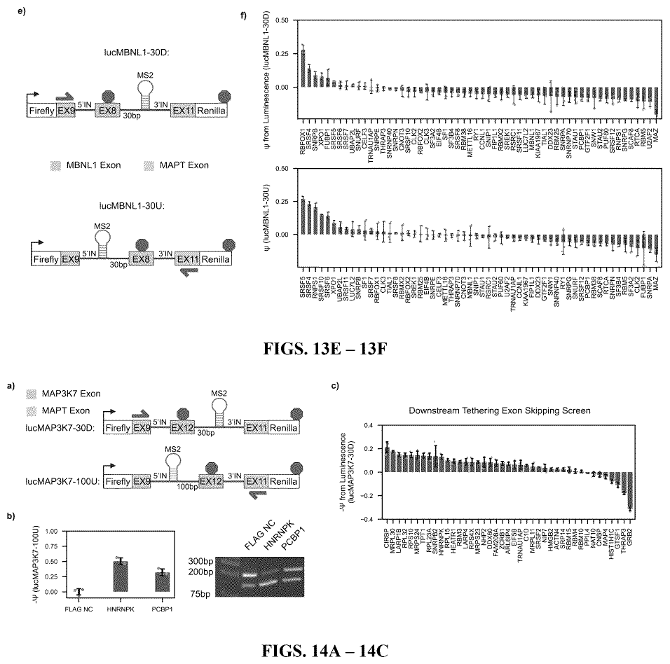

Applicant also tested the final collection of hits with orthogonal exon inclusion reporters. Applicant screened Applicant's hits using lucMAPT reporters containing tethering sites 100 base pairs distal to the splice site instead of 30 base pairs (FIG. 13A). Almost all hits exhibited reduced activity at the increased distance, but proximity-dependence varied by RBP (FIGS. 13B-13D), Supplementary Table 11-12 in Schmok J C (2024), incorporated herein by reference). Finally, Applicant tested all hits with another exon inclusion reporter based around MBNL1 exon 8 (lucMBNL1-FIG. 13E). Though positive control SRSF5 successfully induced exon inclusion, the baseline inclusion rate was perturbed by a small subset of hits, implying some context-dependence of proximity-dependent splicing activity of the tested RBPs (Supplementary Table 13-14 in Schmok J C (2024), incorporated herein by reference, FIG. 13F). Nevertheless, the lucMAPT screens provide one valid context and Applicant continued forward with their findings with the knowledge that Applicant are capturing effects within it.

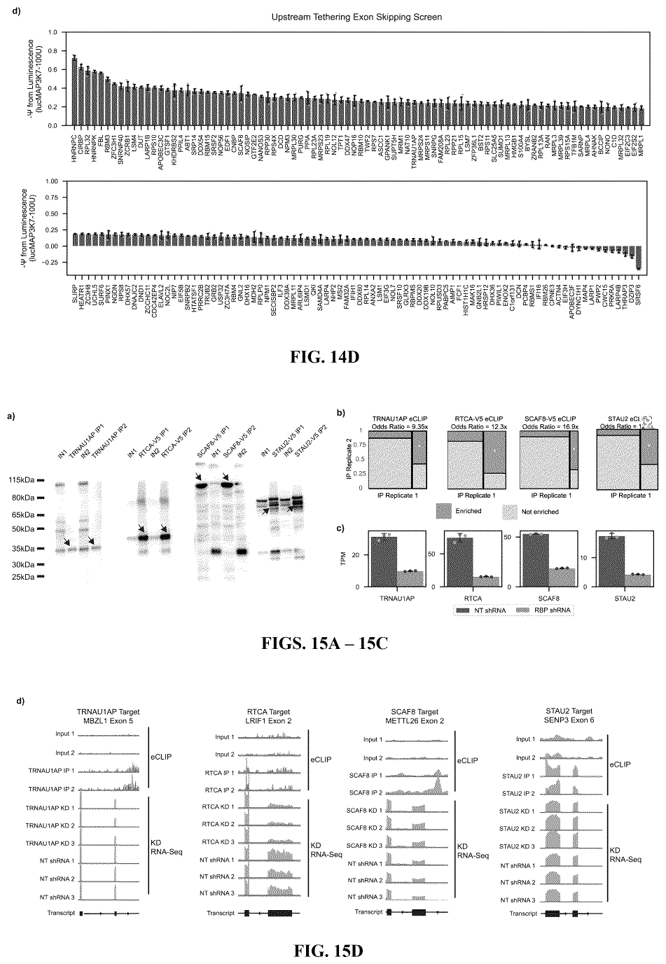

Initially, Applicant had also investigated a complementary approach to identify RBPs that induce exon skipping. Applicant constructed a reporter using the same framework around MAP3K7 exon 12, which is primarily included in HEK293T cells (FIG. 14A). Applicant validated the response of the MAP3K7 reporter to HNRNPK and PCBP1, known activators of exon skipping, using the reporter readout and RNA-level validation when tethered 100 base pairs upstream of the AS exon (FIG. 14B). 22/44 RBPs induced exon skipping when tethered 30 base pairs downstream of the AS exon and 154/194 induced exon skipping when tethered 100 base pairs upstream of the AS exon (FIGS. 14C-14D, Supplementary Table 15-16 in Schmok J C (2024), incorporated herein by reference). The high proportion of hits suggests that recruitment of many proteins may simply act to sterically prevent spliceosome recognition, thus Applicant stopped the skipping screen here and constrained this study to focus on exon inclusion, a more specific molecular task.

### Splicing Events are Modulated by Unexpected Hits

Applicant followed up the screen with endogenous characterization of four hits from the screen which to this point have no established role in AS regulation: STAU2, SCAF8, RTCA and TRNAU1AP. STAU2 is an important protein in neuronal mRNA localization³⁹ that shares 59.9% similarity with paralogue STAU1: a multifunctional RBP with implications for oncogenesis and neurodegeneration^{26,40}. SCAF8 has been previously characterized for roles in selection of distal poly(A) sites and transcriptional elongation, and a selection of genes in the same family are known or predicted to be involved in AS, including SCAF1, SCAF4, and SCAF1141. Although SCAF8 was detected in two previous spliceosomal proteomics experiments, the significance of this finding has not been further investigated^{33,34}. RTCA has been previously characterized for its role in RNA metabolism by catalyzing the conversion of the 3′ phosphate of RNA substrates to a 2′,3′-cyclic phosphodiester⁴². TRNAU1AP is a poorly characterized protein predicted to play a role in Selenocysteine (Sec) biosynthesis and incorporation into selenoproteins⁴³. The four unexpected candidates selected vary widely in structure and currently defined function. To assess whether these are bona fide splicing factors, Applicant applied functional genomics approaches to investigate the activity of the unexpected candidates in cells.

Applicant first interrogated endogenous RNA targets and transcriptome-wide binding sites of the unexpected candidates using enhanced CLIP followed by sequencing (eCLIP) 44 in HEK293T cells. For TRNAU1AP, Applicant performed eCLIP using an IP-grade, specific antibody⁴⁵. For the other unexpected hits which did not have IP-grade antibodies available, Applicant expressed V5-tagged ORFs and performed eCLIP with a validated V5 antibody. Applicant successfully completed immunoprecipitation for all replicates (FIG. 15A). Applicant retrieved enriched windows using the Skipper pipeline⁴⁶ and found them to be reproducible across two independent replicates each for all eCLIP experiments (concordance odds ratio >9× for all experiments, FIG. 15B).

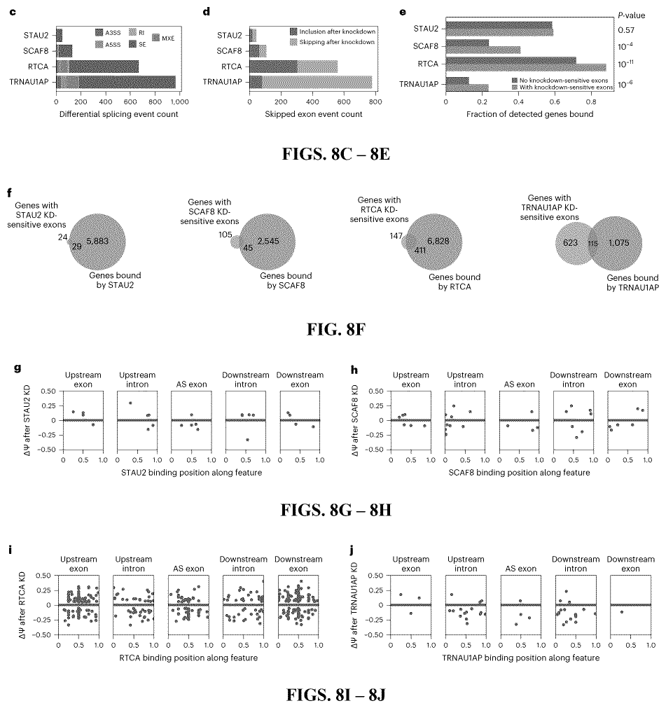

To determine the RNA region preferences of the candidate proteins, Applicant examined the region annotation of all reproducible enriched windows from the eCLIP signals (FIG. 8A). STAU2 reproducible enriched windows were represented most frequently in intronic regions and 3′UTR (also consistent with its known role in RNA localization). The reproducible enriched windows of SCAF8 were frequently near splice junctions, indicative of splicing regulation, with a relatively even distribution of regions otherwise. RTCA displayed widespread binding (>100,000 reproducible enriched binding windows), with a robust preference for coding sequence and 3′UTR (consistent with its role in 3′ RNA processing) binding and a strong under-enrichment of intronic binding when compared to the other candidates. TRNAU1AP binding sites showed a stark preference for intronic binding, resembling the binding patterns of some well-described splicing factors, such as RBFOX2 and HNRNPC28. From region binding alone, Applicant saw patterns in SCAF8 and TRNAU1AP binding that are reflective of known splicing factor binding and patterns among the other candidates that indicate that while the proteins may be able to modulate splicing, they play major roles in other RNA processing steps as well.

Next, Applicant performed motif analysis on the reproducible enriched windows in the eCLIP signal for each of the unexpected hits (FIG. 8B). The top motif for RTCA is part of the known exonic splicing enhancer hexamer sequence 5′-GAAGAA-3′47. The top motif for SCAF8 is a poly(G) run-associated with AS regulation^{48,49}. Overall, examination of the top motif contained within each of the eCLIP signals revealed that RTCA and SCAF8 bind to signals associated with splicing regulation.

To investigate whether these RBPs modulate AS of endogenous RNA, Applicant performed shRNA-mediated knockdown followed by RNA-seq analysis in HEK293T cells with shRNAs specific to these proteins. Knockdowns of all targets were successful, with knockdown of at least 50% as measured by TPM (FIG. 15C). Applicant examined the differential AS events following knockdown and detected differentially spliced events for all knockdowns (FIG. 8C). To simplify characterization, Applicant perform further analysis on differentially spliced events of the skipped exon (SE) category. At least 30 differential SE events were driven by the knockdown of each of these candidates. For RTCA and TRNAU1AP, more than 500 differentially spliced events were detected. Applicant determined the direction of splicing change for each differentially spliced SE event (FIG. 8D). As the initial screens were designed to detect RBPs with the potential to induce exon inclusion, applicant expected to observe splicing events with increased skipping upon knockdown. Applicant observe this trend for TRNAU1AP, indicating that TRNAU1AP is endogenously driving exon inclusion, matching Applicant's prediction from the screens. The other candidates did not display the same trend. Nevertheless, they cannot be eliminated as direct drivers of exon inclusion at this stage, since final AS outcome also captures participation of the unexpected hits in upstream pathways and competitive effects with other splicing factors⁵⁰. The data here indicates that the candidates each play roles in AS regulation of some events, with TRNAU1AP and RTCA modulating many SE events.

To nominate AS exons that could be regulated by direct binding, Applicant integrated findings from eCLIP and RNA-seq. Applicant found that genes containing knockdown sensitive exons are bound at a significantly higher rate than genes lacking knockdown sensitive exons by SCAF8, RTCA, and TRNAU1AP but not by STAU2 (FIG. 8E, FIG. 8F). Although the count of genes containing knockdown sensitive SE events is low for STAU2 in comparison to the count of genes bound, the events in which there is overlap could be directly driven by binding, however this appears to be a more specific than widespread phenomenon, at least in HEK293T cells. RTCA binds to most genes containing knockdown-sensitive SE events, indicating that the binding of RTCA directly drives many splicing changes. TRNAU1AP and SCAF8 both bind a substantial portion of genes with knockdown-sensitive SE events. Splicing modulation of these events may be directly driven by this binding. Some of the non-bound differential splicing events could by driven by their roles in pathways upstream of splicing outcome or could be bound at levels below the detection sensitivity of eCLIP. Altogether, RTCA, SCAF8, and TRNAU1AP appear to directly regulate many SE events through binding, while STAU2 appears to do this in a more limited capacity.

To investigate individual cases of Applicant's candidates directly driving AS modulation through position-dependent binding, Applicant generated maps of knockdown-sensitive splicing events containing nearby binding signal. Applicant found instances of candidate RBP binding to knockdown sensitive exons as well as flanking introns and exons and plotted the center of the reproducible enriched binding windows across these features against the change in exon inclusion level following knockdown (FIGS. 8G-8J). At the few sites with STAU2 binding and STAU2 knockdown-sensitive splicing, no clear pattern emerges, indicating that direct STAU2-mediated splicing change is not a widespread and generalized phenomenon (FIG. 8G). Binding of SCAF8 is distributed throughout AS exons as well as the flanking introns and exons (FIG. 8H). SCAF8 frequently binds at the upstream 5′ splice site of exons that are skipped after knockdown. RTCA binding is prevalent in AS exons, flanking introns, and flanking exons with most prevalent binding in the flanking exons (FIG. 8I). Applicant detected knockdown-sensitive splicing changes in both directions with nearby RTCA binding. TRNAU1AP commonly binds the flanking introns of exons that are skipped after knockdown, with a cluster present at the downstream 5′ splice site, implying that TRNAU1AP binds downstream of alternatively spliced exons and induces exon inclusion (FIG. 8J). This matches the position-dependent effect captured in the initial screen. To visualize specific instances of direct splicing regulation, Applicant generated genome tracks of sample targets with knockdown-sensitive differential splicing and nearby eCLIP signal for TRNAU1AP, RTCA, SCAF8 and STAU2 (FIG. 15D). In summary, Applicant utilized integrated analysis of eCLIP and KD RNA-Seq to identify instances of direct SE modulation by binding of STAU2, SCAF8, RTCA, and TRNAU1AP with SCAF8, RTCA, and TRNAU1AP displaying interesting position-dependent modulatory trends.

### Splicing Protein Enrichment in Pull-Down of Unexpected Hits

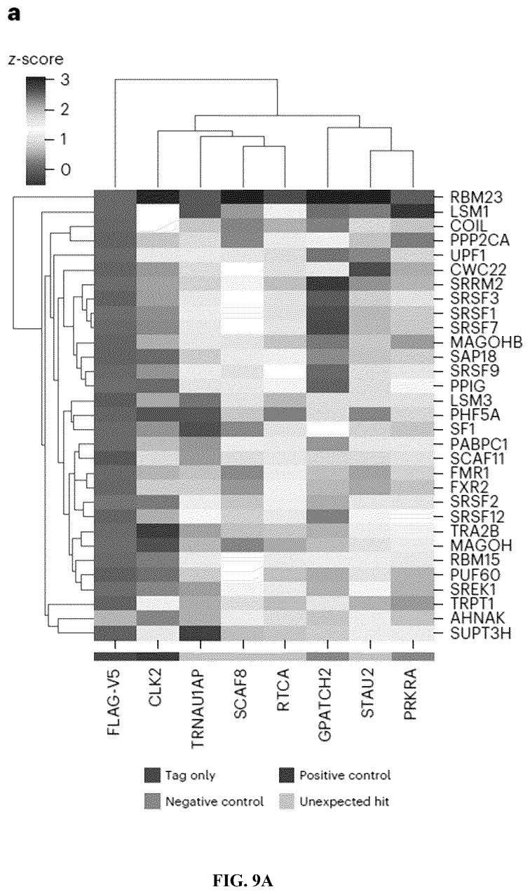

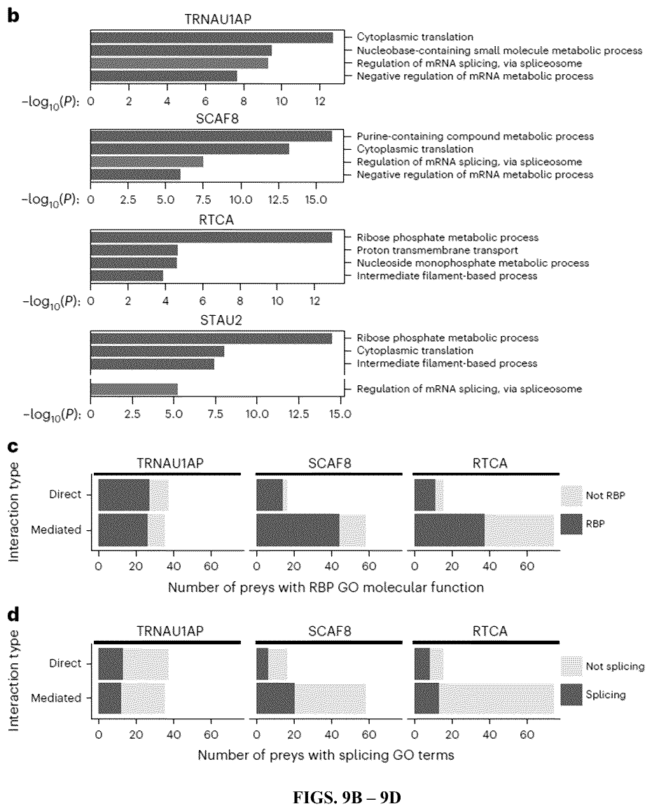

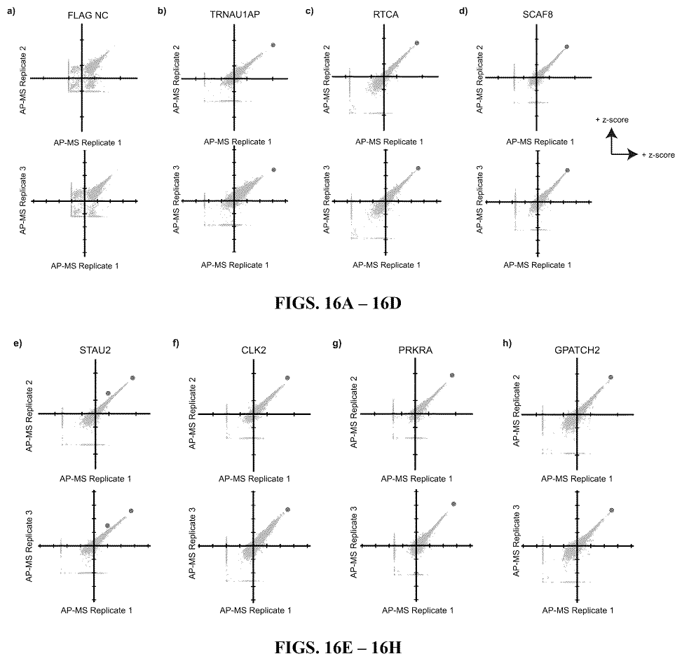

Splicing occurs through assembly and action of complexes consisting of multiple proteins and RNAs, including core spliceosomal components and non-essential splicing factors. To examine if splicing-associated proteins interact with Applicant's candidates, applicant performed affinity purification-mass spectrometry (AP-MS) of V5-tagged TRNAU1AP, RTCA, SCAF8, and STAU2 expressed in HEK293T cells (FIG. 9, Supplementary Table 17 in Schmok J C (2024), incorporated herein by reference). Applicant performed AP-MS in the absence of ribonuclease, allowing the detection of both proteins that interact directly with Applicant's candidates as well as proteins that Applicant's candidates associate with through nearby binding on RNA substrates. Applicant aimed to include these RNA-mediated associations, since mutual binding to the small nuclear RNAs (snRNAs) of the spliceosome or nearby splice sites on mRNA can indicate interactions during splicing. Replicates were highly correlated, and each bait protein was present among the top preys in corresponding samples (FIG. 16). Applicant also performed AP-MS with a known splicing-associated protein, CLK2, a tag-only control (FLAG-V5), and two RBPs from the screens that did not emerge as hits (PRKRA and GPATCH2).

Applicant examined the enrichment of splicing-associated proteins (annotated with GO: 0008380 RNA-splicing, GO: 0005681 Spliceosomal Complex, or any of their child terms) in each of the AP-MS samples that were significantly enriched (Z-score >2) in at least one of the AP-MS samples (FIG. 9A). Setting aside the tag only control, the baits separated into two clusters, one with high enrichment of splicing-associated proteins among the preys and the other with low enrichment. The low enrichment cluster consists of the two non-activating controls and STAU2. Nevertheless, STAU2 is still enriched for interactions with a subset of splicing-associated proteins over the non-targeting controls, potentially due to it performing a limited, auxiliary role in splicing. The high enrichment cluster consists of the known splicing-associated protein CLK2 as well as TRNAU1AP, SCAF8, and RTCA, candidates which also displayed widespread direct modulation of AS of endogenous targets. Overall, the increased enrichment of splicing-associated proteins in the TRNAU1AP, SCAF8, and RTCA AP-MS samples provides supporting evidence towards them performing widespread splicing regulation.

Applicant also performed gene ontology enrichment on the significantly enriched preys as detected by Spectronaut (q value <0.05 and log 2 ratio IP/FLAG >1) with each of the candidates as bait (FIG. 9B). The splicing-associated GO term regulation of mRNA splicing, via spliceosome was among the most highly enriched in the significantly enriched preys pulled down by TRNAU1AP and SCAF8. No splicing-associated GO terms were enriched among the significantly enriched preys pulled down by RTCA. The splicing-associated GO term regulation of mRNA splicing, via spliceosome was enriched in the preys pulled down by STAU2 but was not among the top terms. Following the initial evidence of splicing-associated protein enrichment following TRNAU1AP, SCAF8, and RTCA pulldown, Applicant matched these experiments with ribonuclease-positive conditions as well as matching IgG controls in +/−ribonuclease conditions to distinguish between direct protein-protein interactions and RNA-mediated interactions (FIGS. 9C-9D) 51. Applicant applied a strict p-value cutoff of 0.00000001 to visualize the most specific RBPs and splicing-associated proteins pulled down by each bait. The unfiltered output from follow-up experiments can be found in Supplementary Table 18 in Schmok J C (2024), incorporated herein by reference. Overall, applicant utilized AP-MS to indicate that splicing-associated proteins are enriched following pulldown of TRNAU1AP, SCAF8, and RTCA and to identify the specific modes by which these proteins interact with RBPs and splicing-associated proteins.

### Alternative Splicing Modulation by TRNAU1AP

Due to strong evidence across the eCLIP, KD RNA-Seq, and AP-MS data indicating the activity of TRNAU1AP as a splicing factor, Applicant examined the protein in further detail. Applicant first investigated the finding that most genes with TRNAU1AP-knockdown sensitive skipped exon events did not contain reproducible enriched binding windows from the eCLIP data. Applicant considered the hypothesis that some of this effect could be explained by TRNAU1AP indirectly regulating splicing events through modulating the splicing of other splicing factors. This multi-layered control of splicing has been shown in the recently characterized splicing factor DAP352, as well as in the SR-family of splicing factors²⁰. To investigate this, applicant examined the top differentially expressed and differentially spliced genes with RNA splicing GO terms (splicing-associated genes) following TRNAU1AP knockdown.

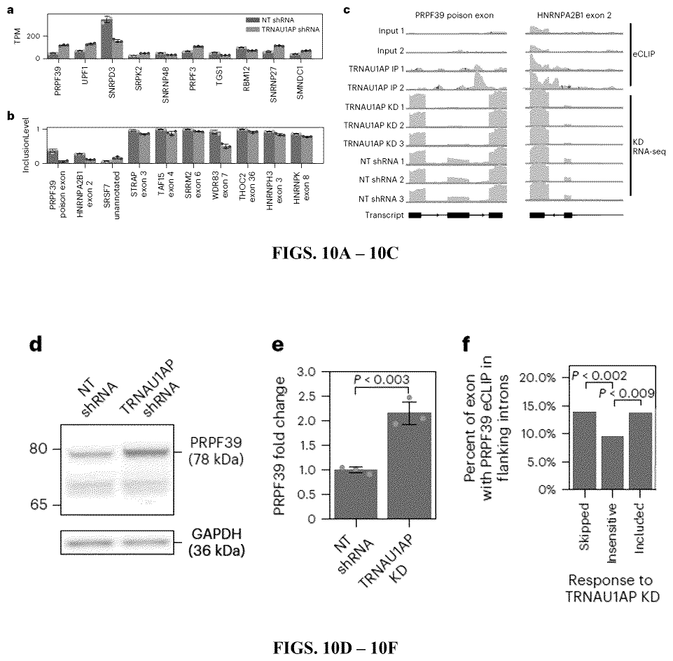

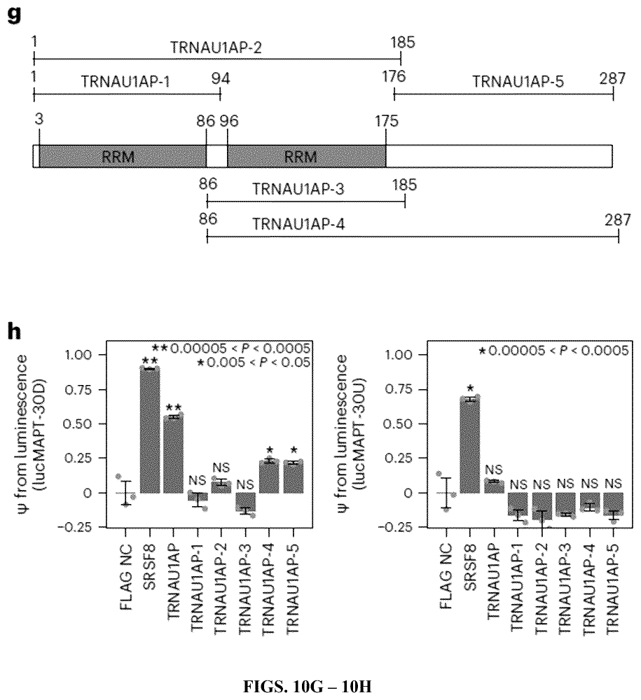

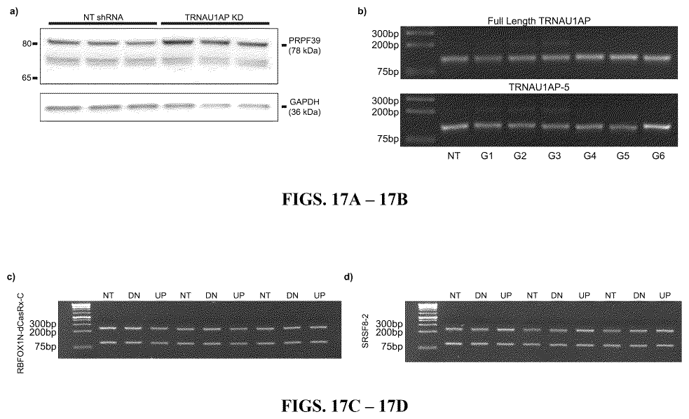

The top differentially expressed splicing-associated gene was PRPF39 (FIG. 10A), and the top two differentially spliced splicing-associated genes were PRPF39 (at an unannotated poison exon) and HNRNPA2B1 (at exon 2, responsible for isoform switching between HNRNPA2 and HNRNPB1) (FIG. 10B). In TRNAU1AP knockdown, presence of the PRPF39 poison exon is virtually eliminated and PRPF39 TPM increases from 46.06+/−3.62 to 117.34 +/−5.06 (mean+/−standard deviation). TRNAU1AP binds in the intron downstream on this poison exon (FIG. 10C, left). Applicant performed western blots to validate that the increase in PRPF39 expression following TRNAU1AP KD is reflected at the protein level and detected a two-fold increase in HEK293T cells (FIGS. 10D-10E and FIG. 17A). Due to the extent of poison exon elimination in the knockdown condition, TRNAU1AP appears to be the primary driver of poison exon-mediated expression control of PRPF39 in HEK293T cells. As an initial investigation to test the hypothesis of PRPF39 acting as a direct effector for certain TRNAU1AP KD-sensitive AS events, Applicant analyzed PRPF39 eCLIP signal in HepG2 cells generated by the ENCODE consortium⁴⁵. Applicant found that PRPF39 reproducible enriched binding windows are prevalent in a significantly higher percentage of introns flanking TRNAU1AP-sensitive exons than TRNAU1AP-insensitive exons, supporting the hypothesis (FIG. 10F). Applicant also examined another TRNAU1AP-sensitive splicing factor exon: HNRNPA2B1 Exon 2, which also containing TRNAU1AP binding sites in the downstream intron, and is virtually eliminated in TRNAU1AP KD (FIG. 10B. FIG. 10C, right). This implicates TRNAU1AP as the primary driver of isoform switching of HNRNPA2B1 in HEK293T cells. Here Applicant showed that TRNAU1AP binds to the downstream intron of and drives the inclusion of exons in PRPF39 and HNRNPA2B1, which likely drives further widespread splicing changes.

To identify the effector domain bestowing TRNAU1AP's ability to drive exon inclusion, applicant then performed a series of truncation experiments. Applicant cloned truncations (FIG. 10G) into MCP-fusions using the same backbone as the RBP library in the initial tethering screen. Applicant co-transfected MCP-fused TRNAU1AP truncations with both splicing reporters, attempting to identify the region of the protein sufficient to drive the downstream-only effect captured in the screen (FIG. 10H). The C-terminal domain captured in truncations TRNUA1AP-4 and 5 appears to be responsible for most, but not all, of the exon inclusion driving activity of the full-length protein. This allowed us to build a domain model that matches the standard simplified model of an RBP, consisting of independent and separate effector and binding domains, in this case, an RNA-binding RRM containing domain at the N-terminus and an exon inclusion activating effector domain at the C-terminus.

To ensure that the exon including capacity of TRNAU1AP and its C-terminal effector domain is not dependent on the MS2-MCP interaction, applicant cloned CRISPR artificial splicing factors by fusing TRNAU1AP-5 and full-length TRNAU1AP to catalytically dead Cas13d. Applicant co-transfected these artificial splicing factors with a version of the lucMAPT splicing reporter lacking MS2-stem loops, along with individual gRNA plasmids targeting the introns upstream and downstream of the alternatively spliced exons (FIG. 10I). Both full-length TRNAU1AP and TRNAU1AP-5 significantly drove exon inclusion as measured by the tethering-free reporter when co-transfected with gRNAs targeting downstream of the alternatively spliced exon, but not with those targeting upstream (FIGS. 10J-10K and FIG. 17B). These results are consistent with the downstream-only result from the tethering assays and show that the ability of TRNAU1AP and its C-terminal effector domain to induce exon inclusion are independent of the MS2-MCP interaction. In summary, Applicant show that TRNAU1AP participates in splicing co-regulatory networks and drives exon inclusion through its C-terminal effector domain.

### Employing Identified Domains in Artificial Splicing Factors

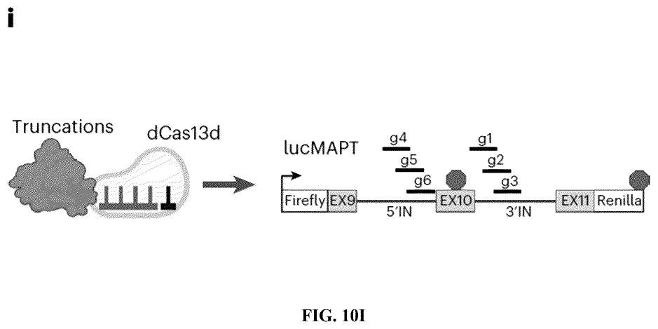

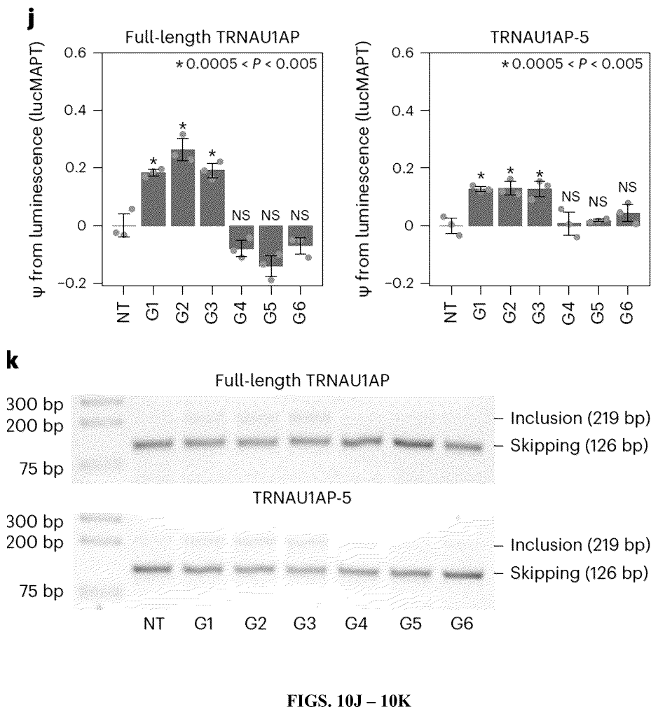

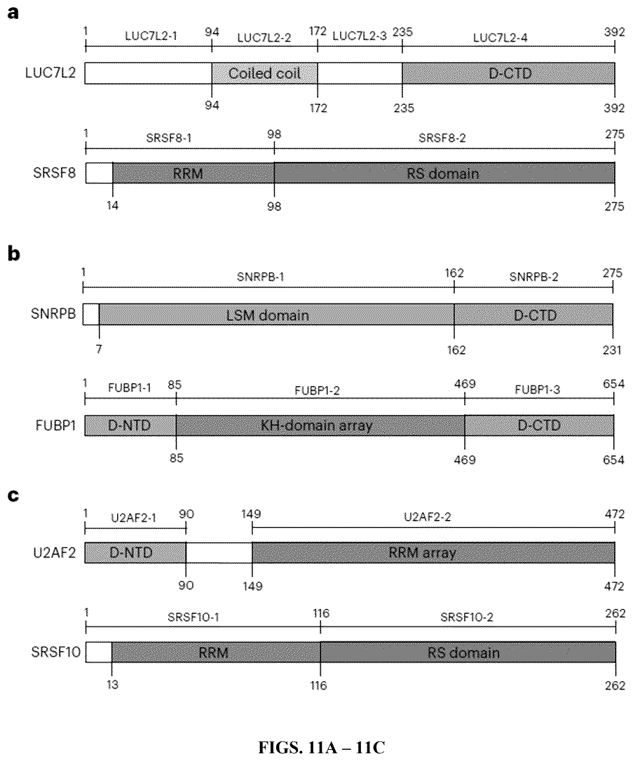

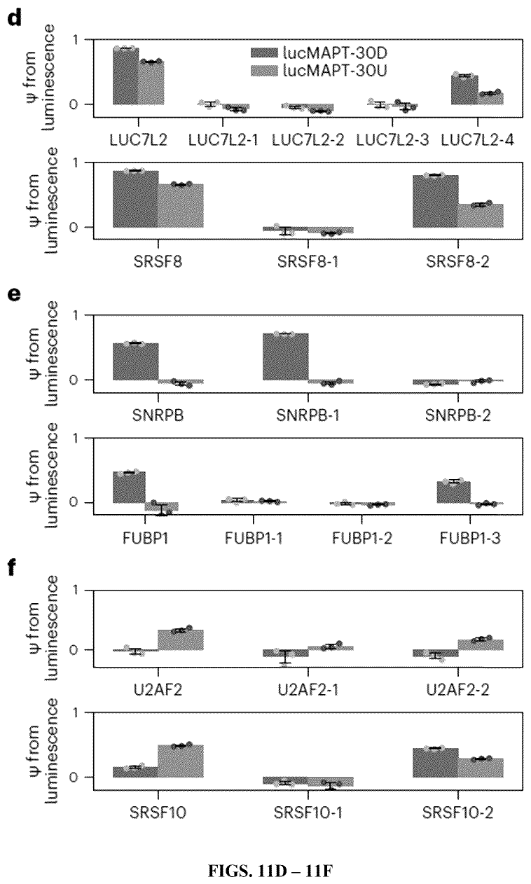

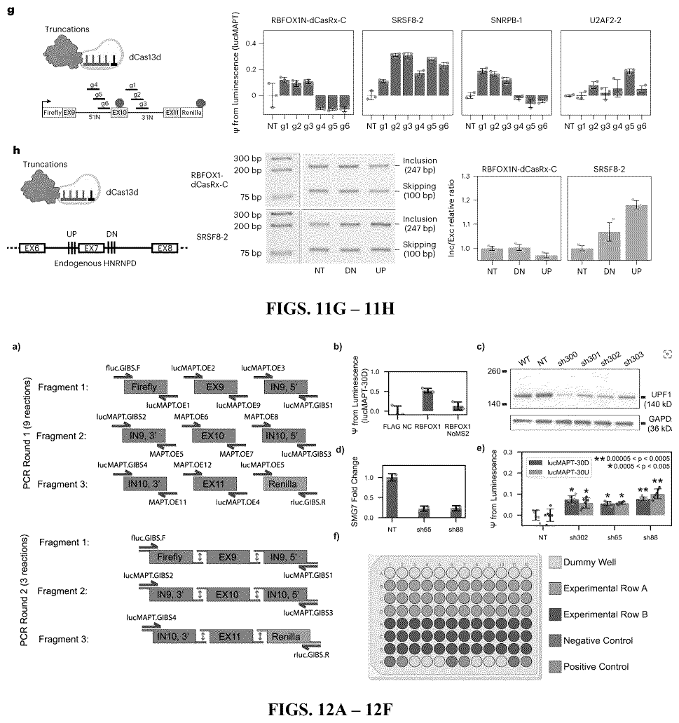

Motivated by Applicant's results articulating that TRNAU1AP or its domain can be useful in artificial splicing factors, Applicant returned to the original list of top RBPs that altered splicing of Applicant's reporter construct and tested various protein truncations of these with the aim of determining minimal splice activating domains to repurpose for artificial splicing factors. LUC7L2 and SRSF8 were selected as strong hits that activated splicing both upstream and downstream of the alternative exon (FIG. 11A). SNRPB and FUBP1 were selected as strong hits that activated lucMAPT-30D only (FIG. 11B). U2AF2 and SRSF10 were selected as strong hits that primarily activated exon inclusion when tethered upstream (FIG. 11C). Applicant designed and cloned truncations based on domain structure, assuming modularity of RBPs where effector and binding domains are separate and independent.

Selected truncations were fused to the MS2-coat protein using the same backbone and conditions as the RBP-MCP library (FIGS. 11D-11F). LUC7L2-4 recapitulated some of the activity of its full-length counterpart, however at substantially lower strength, implying important contributions from the other domains. SRSF8-2, the RS-domain of the protein, captured much of the activity of SRSF8. FUBP1-3 captured much of the activity of full-length FUBP1, at a drastically reduced size. SNRPB-1 captured all the activity of SNRPB. Interestingly, SRSF10-2, the RS-domain of SRSF10, displayed a different modulation pattern than the full-length protein, where a stronger effect was seen when tethered downstream of the alternatively spliced exon, more in line with all other tested SRSF proteins. U2AF2-2 was the most successful truncation of the proteins that only activated lucMAPT-30U.

Applicant constructed CRISPR-based artificial splicing factors by fusing the truncations that most successfully activated the tethering reporter to catalytically dead Cas13d. These were tested with an MS2-free luciferase splicing reporter and compared to the recently reported RBFOX1N-dCasRx-C artificial splicing factor¹⁹ (FIG. 11G). As expected, RBFOX1N-dCasRx-C activated the reporter only when targeting sites downstream of the alternatively spliced exon, with a maximal ψ of 11.87% with g1. The SRSF8-2 based artificial splicing factor activated the reporter at all positions, with a maximal ψ of 31.34% with g2. The SNRPB-1 based artificial splicing factor activated the reporter only when targeting downstream of the alternatively spliced exon as for RBFOX1N-dCasRx-C, but with a greater maximal ψ of 19.15% with g1. The U2AF2-2 based artificial splicing factor did not show activation only with upstream gRNAs as expected, although activation was maximized with upstream guide g5 at 18.60%. Altogether, the SNRPB-1 artificial splicing factor directly outperformed RBFOX1N-dCasRx-C, the SRSF8-2 artificial splicing factor provided a stronger tool with reduced position dependence, and the U2AF2 artificial splicing factor introduced a tool with upstream position association.

Activation of endogenous exon inclusion has remained challenging for the field, as the current solutions with ASOs are to block splicing repressor sites, which is not generalizable to exons that lack these. Applicant employed a CRISPR artificial splicing factor based on Applicant's strongest activation domain, SRSF8-2, against an endogenous exon. Applicant targeted exon 7 of HNRNPD in HEK293T cells, selected for its high expression for facile readout and endogenous inclusion rate of roughly 50% for perturbation detection. Applicant compared Applicant's SRSF8-2 artificial splicing factor to the previous RBFOX1N-dCasRx-C artificial splicing factor by co-transfecting each with plasmids containing arrays of 3 gRNA sequences separated by repeats that are processed by Cas13d into independent guides. RBFOX1-dCasRx-C was not able to activate endogenous HNRNPD Exon 7 inclusion with either of the gRNA arrays, while SRSF8-2 was able to with both arrays, especially the upstream array (FIG. 11H and FIGS. 17C-17D). Exon 7 of HNRNPD appears to be most sensitive to inclusion-driving perturbation with effector domains guided to the upstream 3′ splice site, which is incompatible with the downstream-only effect of RBFOX1-dCasRx-C but can be driven by SRSF8-2, exemplifying the importance of its generalizability. Furthermore, the stronger SRSF8-2 appeared to cross an activation threshold when guided to the downstream 5′ splice site, while the weaker RBFOX1-dCasRx-C did not. In summary, Applicant's tethering assay and reporter system also allowed us to identify small and potent effector domains that Applicant used to improve synthetic splicing modulatory proteins.

### Materials and Methods

Generation of Expression Plasmids for MCP and dCas13d-Fused RBPs and RBP Truncations

The majority of ORF clones were obtained in pENTR vectors from the CCSB human ORFcome collection⁵⁸ (Dana-Farber Cancer Institute) or the DNASU Plasmid Repository (Arizona State University). For truncations, domain structures were determined using InterProScan⁵⁹ on the amino acid sequence of the full-length protein and informed truncation design. Truncations and ORFs that were ordered in standard expression vectors were amplified by PCR (Phusion polymerase, NEB) with oligonucleotide primers containing attB recombination sites and recombined into pDONR221 using BP clonase II (Thermo Fisher). ORFs were then recombined into one of two custom pEF DEST51 destination vector (Thermo Fisher). For MCP-fusions, the destination vector is engineered to direct expression of the ORFs as fusion proteins with a V5 epitope tag and MCP appended C terminally and under the control of the EF1-alpha promoter to create ORF-V5-MCP constructs. For dCas13d-fusions, the MCP is simply replaced with dCas13d for the generation of ORF-V5-dCas13d constructs. Supplementary Table 19 in Schmok J C (2024), incorporated herein by reference contains sequences of both destination vectors. The identity of all cDNA clones was verified by Sanger sequencing. Plasmid libraries are available on Addgene (155390-156159). Supplementary Table 1 in Schmok J C (2024), incorporated herein by reference lists all ORFs and relevant information.

### Cell Lines

Lenti-X HEK293T cells were purchased from Takara Bio and were not further authenticated. Cells were routinely tested for mycoplasma contamination with a MycoAlert mycoplasma test kit (Lonza) and were found negative for mycoplasma.

Generation of Constructs lucMAPT Reporter: Reporter was first constructed through a three-fragment Gibson

Assembly using a homebrew enzyme mix (OpenWetWare). Fragments were generated by performing PCR on sub-fragments to generate complementary overhangs followed by annealing, amplification, and agarose gel extraction. The first fragment consists of Firefly luciferase, MAPT Exon 9 and the 5′-most 500 base pairs of MAPT Intron 9. The second fragment consists of the 3′-most 500 base pairs of MAPT Intron 9, modified MAPT Exon 10, and the 5′-most 500 base pairs of MAPT Intron 10. The third fragment consists of the 3′-most 500 base pairs of MAPT Intron 10, MAPT Exon 11, and Renilla luciferase. Luciferase ORFs were cloned from plasmids used in Applicant's lab's previous work¹⁶. MAPT Exons were ordered as synthetic oligonucleotides. MAPT Intronic Sequences were amplified from genomic DNA isolated from Lenti-X HEK293T cells. All PCR was performed using KAPA HiFi HotStart ReadyMix (Roche #7958935001). The assembly strategy is summarized (see FIG. 12A).

lucMAPT-MS2 Reporters: MAPT Exon 10 and the flanking 100 intronic base pairs in either direction from the splice sites were removed from the construct and replaced with a cloning site containing BamHI and EcoRI cut sites through PCR followed by two-fragment Gibson Assembly to generate a customizable backbone. Inserts containing MAPT Exon 10, the flanking 100 base pairs, and the MS2 stem-loop sequence in the desired position were cloned into this backbone through one-fragment Gibson Assembly into pcDNA3.1 (−) Mammalian Expression Vector (ThermoFisher #V79520) to construct lucMAPT-MS2 reporters. Inserts containing other AS exons and flanking sequences are used to generate other reporters used. Sequences of reporters are found in Supplementary Table 19 in Schmok J C (2024), incorporated herein by reference.

### Luciferase Reporter Screens

Reverse Transfection: 96-well Solid Black Flat Bottom Polystyrene TC-treated Microplates (Corning #3916) were coated with 75 μL Poly-D-lysine hydrobromide (Sigma-Aldrich #P6407-5 MG) dissolved in water at 1 g/L and further diluted 1:5 in 1×DPBS (Corning #21-031-CV) overnight in a tissue culture incubator. Plates were rinsed 2× with 1×DPBS and dried. A 1:1 mix of lucMAPT-MS2 reporter and an ORF-V5-MCP construct with a total of 100 ng DNA were added to a mixture of Lipofectamine 3000 and P3000 reagents (ThermoFisher #L3000001) diluted in Opti-MEM Reduced Serum Media (Gibco #31985062) and incubated for 15 minutes. The mixture of DNA and transfection reagent was transferred to the PDL-coated 96-well plate. 75 μL of Lenti-X HEK293T cells were plated at a concentration of 266,666 cells/mL. Transfection was incubated for 48 hours in a standard tissue culture incubator.

Dual-Luciferase Readout: Luminescence was generated using the Dual-Glo Luciferase Assay System (Promega #E2980). Cells were removed from the incubator to cool to room temperature for 30 minutes. 75 μL Dual-Glo Luciferase Reagent was added directly to cells and thoroughly mixed using a Microplate Genie Plate Shaker (Scientific Industries). The reaction was briefly centrifuged and allowed to incubate at room temperature for 10 minutes. Luminescence was measured using a Spark Multimode Microplate Reader (Tecan) with a 500 ms signal interaction time at room temperature. The same process was repeated for Renilla luciferase luminescence using the Dual-Glo Stop & Glo Reagent.

Statistical Analysis: Relative ψ values were calculated as described in FIG. 6B using the pandas library in Python v3.10.1160. All plots generated from Python were generated using JupyterLab 4.04. Significance between candidate and negative control conditions was assessed by calculating p-value through a one-tailed independent t-test using the ttest_ind function in scipy⁶¹.

### RNA-Level Validation of Luciferase Screens

Transfection was performed as described for the luciferase reporter screens, using standard 96-well tissue culture plates (Costar #3596). RNA was isolated from cells using the Direct-zol RNA Miniprep Kit (Zymo Research #R2052). cDNA was generated using the ProtoScript II First Strand cDNA Synthesis Kit (Promega #E6560L). cDNA was amplified using GoTaq Green Master Mix (Promega #M7122) and primers designed for an amplicon stretching from MAPT Exon 9 to the Renilla Luciferase ORF. Amplicons were run through a 3% SeaKem Agarose Gel (Lonza #5004) at 100V for 25 minutes.

Statistical Analysis: Relative band intensity was calculated using the Gel Analyzer feature in ImageJ v1.53k software⁶². Significance between candidate and negative control conditions was assessed by calculating p-value through a one-tailed independent t-test using the ttest_ind function in scipy⁶¹.

### Gene Ontology Analysis

Metascape v3.5 was used for GO analysis⁵⁶. Custom enrichment analysis for GO Biological Processes was performed using an appropriate set of background genes. biomaRt v2.50.3 was used to identify genes matching specific GO terms from gene lists⁶³. Applicant used biomaRt to generate a list of splicing associated genes by selecting genes annotated with GO: 0008380 RNA-splicing, GO: 0005681 Spliceosomal Complex, or any of their child terms.

### Generation of Samples Overexpressing V5-Tagged RBPs

HEK293T cells were plated in 10 cm plates at 10% confluency. 28 ng plasmid DNA encoding the V5-tagged RBPs were added to a mixture of Lipofectamine 3000 and P3000 reagents (ThermoFisher #L3000001) diluted in Opti-MEM Reduced Serum Media (Gibco #31985062) and incubated for 15 minutes. The mixture of DNA and transfection reagent was transferred to the plated cells. Cells were collected 48 hours later and washed with 10 mL DPBS. Samples to be used for eCLIP were UV-cross-linked (400 mJ cm⁻², 254 nm). Cells were resuspended in 1 mL DPBS. Samples were centrifuged at 4 C, 18,000×g for 1 minute. Supernatant was removed and cells were flash frozen in dry ice before storage at −80 C until experimentation.

eCLIP Library Preparation and Sequencing

eCLIP was performed as per standard operating procedures⁴⁴. Antibodies used are listed in Supplementary Table 20 in Schmok J C (2024), incorporated herein by reference. For V5-tagged eCLIPs, overexpression samples were generated as described herein. Samples for endogenous eCLIP were generated using the same procedure without transfection. Two replicates were generated for each experiment. Pellets were lysed, and lysates were subjected to sonication and RNase I to fragment RNA. Ninety-eight percent of each lysate was immunoprecipitated using either V5 (Bethyl A190-120A) or TRNAU1AP-specific (GeneTex GTX121631) antibodies, and the remainder was stored for preparation of a SMInput library. 10 ug antibody was used per sample. Pulled-down RNA fragments were dephosphorylated, and 3′-end ligated to an RNA adaptor. Immunoprecipitates and SMInputs were run on an SDS-polyacrylamide gel and transferred to a nitrocellulose membrane. Membrane regions from the RBP size to that size plus 75 kDa were excised, and RNA was released with proteinase K. SMInput samples were then dephosphorylated, and 3′-end ligated to an RNA adaptor. All samples were reverse transcribed with SuperScript III Reverse Transcriptase (LifeTech). cDNAs were ligated to a DNA adaptor at the 5′-end. cDNA was quantified by qPCR and amplified to 100-500 fmol of library using Q5 PCR Master Mix (NEB). Sequencing was performed using the NovaSeq 3000 platform, with a targeted number of single-ended reads of 40M per sample.

Computational Analysis of eCLIP Data

Computational analysis of eCLIP data was performed using the default settings of Skipper resources available on Github [https://github.com/YeoLab/skipper]. Reads were mapped to human genome assembly GRCh38⁶⁴. For V5-tagged eCLIPs, reproducible enriched windows were first found following transfection and eCLIP of a V5-FLAG negative control plasmid and added to the blacklist file to reduce spurious enrichment from V5 binding to RNA.

shRNA Lentiviral Production, Transduction, and Sequencing

To generate lentiviral particles for RBP knockdown, Applicant seeded 500,000 HEK293T cells/well in 6-well plates. After 24 h, cells in each well were transfected with 500 ng sequence-verified shRNA plasmid (pLKO.1, Supplementary Table 21 in Schmok J C (2024), incorporated herein by reference) and packaging plasmids (50 ng pMD2.G: Addgene 12259; 500 ng psPAX2: Addgene 12260; both gifts from D. Trono, École polytechnique fédérale de Lausanne) using Lipofectamine 3000 (Thermo Fisher). Transfection media was replaced with 2.5 mL fresh media after 6 hours. Virus-containing medium was collected 48 h later, replaced with 2.5 mL fresh media, and collected again a further 24 h later. Virus-containing media were pooled and stored at −80 C until transduction.

For lentiviral transduction, 500,000 HEK293T cells were seeded per well in each well of a 6-well tissue culture plate. After 24 h, media was replaced with 2 mL virus containing media supplemented with 16 ug polybrene. Applicant replaced the virus containing media with fresh media 24 h later. 24 h after this, media was replaced with fresh media containing 3 μg/mL puromycin. Cells were either given fresh puromycin-containing media or passaged every 48 h and expanded to 10 cm plates. Cells were pelleted and flash-frozen once all replicates for a given construct had reached 70% confluency or higher.

Total mRNA was extracted from samples using the Direct-zol RNA Miniprep Kit (Zymo Research). RNA-quality was verified using the Tapestation 3000 (Agilent). Library preparation was performed using the Stranded mRNA Prep, Ligation kit (Illumina). Sequencing was performed using the NovaSeq 3000 platform, with a targeted number of paired ended reads of 60M per sample. Read counts and uniquely mapped reads were verified after STAR v2.6.7a alignment.

### Differential Expression Analysis

Differentially expressed genes were detected from RNA-seq data using DeSeq2⁶⁵. Applicant only considered genes expressed with TPM>10 in the control sample.

### Differential Splicing Analysis

Differential AS events were detected using rMATS 4.0.266. Splicing events were identified as significantly differentially spliced if the absolute value of inclusion level difference was detected as greater than 5%, and with an FDR less than 5%. Applicant only considered differential splicing events with a sum of >=150 reads across all conditions.

Integrated Analysis of eCLIP and shRNA KD Followed by RNA-Seq Data

The fraction of knockdown-sensitive or knockdown-insensitive genes containing binding sites from eCLIP was calculated using the number of genes expressed with TPM>=10 from the eCLIP sized-matched input as the denominator.

Binding position relative to knockdown sensitive exons is visualized as the midpoint of the significantly enriched window. For events where multiple significantly enriched windows were present in a single feature, the midpoint of the median window is displayed.

### Western Blots

Cells were lysed in lysis buffer (see eCLIP protocol) on ice for 15 minutes and sonicated for 5 minutes. Lysates were centrifuged at 15,000 g for 10 minutes at 4° C. to pellet debris and transferred to a clean tube. Total protein concentration was quantified using the Pierce BCA Protein Assay Kit (Thermo 23225). For gel electrophoresis, 20 μg was loaded per well onto 4-12% Bis-Tris gels and subsequently transferred to PVDF membranes. Membranes were blocked in 5% milk in TBST solution for 60 minutes at room temperature. Primary antibodies for UPF1 (Cell Signaling Technology D15G6, 1:1000), PRPF39 (Invitrogen PA5-21627, 1:1000) and GAPDH (Millipore MAB374, 1:10,000) were diluted in 5% milk in TBST and probed overnight at 4° C. . . . Secondary antibodies (Goat Anti-Rabbit IgG, HRP Linked-Cell Signaling Technology 7074 and 800CW Goat Anti-Mouse IgG-Licor 926-32210) were diluted at 1:2000 in 5% milk in TBST and probed for 120 minutes at room temperature.

### Affinity Purification Mass Spectrometry

HEK293T cells overexpressing V5-tagged RBPs were generated as described herein. Cells were lysed, and affinity purified using 10 μg per sample of a V5 specific antibody. Briefly, the cell lysates with antibody were incubated with magnetic beads overnight in the cold room. 5 μL of 10 mg/mL RNase A was added to ribonuclease-positive conditions at this step. Supernatants were removed, beads were washed 4 times with NP-40 buffer, twice in Buffer 2 (50 mM Tris [pH 7.5], 150 mM NaCl, 10 mM MgCl2, 0.05% NP-40, and 5% glycerol); and twice in Buffer 3 (50 mM Tris [pH 7.5], 150 mM NaCl, 10 mM MgCl2, and 5% glycerol). After the last wash, the wash buffer was aspirated completely, and the beads were resuspended in 80 μl trypsin buffer (2 M Urea, 50 mM Tris [pH 7.5], 5 μg/ml trypsin) to digest the bound proteins at 37° C. for 1 h with agitation. The beads were centrifuged at 100×g for 30 sec, and the partially digested proteins (the supernatant) were collected. The beads were then washed twice with 60 μl Urea buffer (2 M Urea, 50 mM Tris [pH 7.5]). The supernatant of both washes was collected and combined with the partially digested proteins (final volume is 200 μl). After brief centrifugation, the combined partially digested proteins were cleared from residual beads. 80 ul of these partially digested proteins were used and disulfide bonds were reduced with 5 mM dithiothreitol (DTT), and cysteines were subsequently alkylated with 10 mM iodoacetamide. Samples were further digested by adding 0.5 μg sequencing grade modified trypsin (Promega) at 25° C. After 16 h of digestion, samples were acidified with 1% formic acid (final concentration). Tryptic peptides were desalted on C18 StageTips according to (Rappsilber et al., 2007) and evaporated to dryness in a vacuum concentrator and reconstituted in 15 μl of 3% acetonitrile/2% formic acid for LC-MS/MS.

LC-MS/MS analysis was performed on a Q-Exactive HF. 5 μL of total peptides were analyzed on a Waters M-Class UPLC using a 25 cm Thermo EASY-Spray column (2 um, 100A, 75 um×25 cm) coupled to a benchtop ThermoFisher Scientific Orbitrap Q Exactive HF mass spectrometer. Peptides were separated at a flow rate of 400 nL/min with a 100 min gradient, including sample loading and column equilibration times. Data was acquired in data-independent (DIA) mode for initial experiments and data-dependent (DDA) mode for follow-up experiments. DIA MS1 spectra were measured with a resolution of 120,000, an AGC target of 5e⁶ and a mass range from 350 to 1650 m/z; 34 isolation windows of 38 m/z were measured at a resolution of 30,000, an AGC target of 3e6, normalized collision energies of 22.5, 25, 27.5, and a fixed first mass of 200 m/z. DDA MS1 spectra were measured with a resolution of 120,000, an AGC target of 3e⁶ and a mass range from 300 to 1800 m/z; MS2 spectra were measured at a resolution of 15,000, an AGC target of 1e5, a TopN of 12, an isolation window of 1.6 m/z, and a mass range from 200 to 2000 m/z.

Proteomics raw data was analyzed by Spectronaut v16.0⁶⁷ (Biognosys) using a UniProt database (Homo sapiens, UP000005640), and MS/MS searches were performed under BGS factory settings. UniProt GO term annotations (downloaded Jan. 14, 2022) were used for the differential enrichment analysis conducted by the Spectronaut software. Spectromine v4.2.230428.52329 was used to analyze proteomics data in follow-up experiments using the same UniProt databases and default parameters. Preys identified in both the RNase treatment and non-treatment IPs for a particular bait were called “direct interactors” and preys identified in only RNase non-treatment were called “RNA-mediated interactors.”

Modulation of Splicing with dCas13d Fusions

Transfection was performed as described for the luciferase reporter screens. The plasmid DNA transfected consisted of 10 ng lucMAPT Reporter DNA, 45 ng gRNA plasmid, and 45 ng dCas13d-RBP fusion. Dual-luciferase readout was collected as described for the luciferase reporter screens. gRNA sequences were designed using the cas13design tool^{68,69}. Transfection for modulation of endogenous targets was performed in 24-well plates with 250 ng gRNA plasmid DNA and 250 ng dCas13d-RBP fusion.

RNA-seq and eCLIP-seq data of this study are available at NCBI-GEO (accession code GSE232599)⁷⁰.

### Experimental Discussion

Applicant developed tethering assays and used these to assess the ability of 718 RBPs to induce exon inclusion following recruitment nearby an alternatively spliced cassette exon. Of the 718 RBPs evaluated, 58 reliably enhanced inclusion. 47 of these 58 were annotated with splicing-associated GO terms, and 11 of these were previously unknown as performing any role in AS. Applicant further applied Applicant's assays for technology development by using them to rapidly test exon inclusion activation domains identified from the top candidates for use in engineered splicing factors. By fusing these identified domains to catalytically dead Cas13d, Applicant built CRISPR-based artificial splicing factors that are smaller, more potent, and less restricted than current technologies. Applicant's tethering assays served as fast, scalable, and reliable platforms for both applications.

Applicant employed eCLIP, AP-MS, and shRNA KD followed by RNA-seq to endogenous TRNAU1AP, SCAF8, RTCA and STAU2, and excitingly provided evidence for regulation of splicing outcomes. Applicant further implicated TRNAU1AP as a multilayered regulator of splicing that also acts in splicing regulatory networks by modulating the splicing of other splicing factors. Applicant performed AP-MS in ribonuclease-free conditions and detected splicing-associated proteins following pull-down of TRNAU1AP, RTCA, and SCAF8, further supporting their role in splicing. Findings here are limited by the sensitivity and specificity of the assays chosen, as well as potential tissue specificity of effects on splicing of the chosen proteins. Future work should investigate the role of these proteins on splice site selection in orthogonal models and employ further validation approaches such as minigene assays of specific splicing events and co-IP western blots to validate interaction partners.

Furthermore, the functional consequences of splicing modulation by TRNAU1AP, SCAF8, RTCA, and STAU2 in health and disease remain to be investigated. The splicing regulatory network formed by TRNAU1AP and PRPF39 deserves further investigation. TRNAU1AP and PRPF39 were recently identified as a co-dependency module that is selectively essential in cells carrying mutational signatures of DNA mismatch repair⁵³. The interaction of TRNAU1AP regulating PRPF39 expression through poison exon inclusion described here provides a mechanistic hypothesis for this finding. Furthermore, both genes are prognostic markers in a variety of cancer types⁵⁴.

Applicants SNRPB-1 artificial splicing factor maintained the downstream targeting specificity of the prior RBFOX1N-dCasRx-C artificial splicing factor, but with higher potency and a reduced size. Applicant also identified exon activation domains with different specificity requirements. Applicant's U2AF2-2 artificial splicing factor has maximum potency when targeted upstream of an AS exon, while Applicant's SRSF8-2 artificial splicing factor is the strongest thus far and maintains potency with proximity to the AS exon independent of orientation. This orientation-independence proved important in Applicant's targeting of endogenous HNRNPD Exon 7, where SRSF8-2 successfully activated exon inclusion and RBFOX1N-dCasRx-C did not.

### EQUIVALENTS

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

Unless otherwise defined, all technical and scientific terms used herein have the same meaning as commonly understood by one of ordinary skill in the art to which this disclosure belongs. All nucleotide sequences provided herein are presented in the 5′ to 3′ direction.

The embodiments illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure.

Thus, it should be understood that although the present disclosure has been specifically disclosed by specific embodiments and optional features, modification, improvement and variation of the embodiments therein herein disclosed may be resorted to by those skilled in the art, and that such modifications, improvements and variations are considered to be within the scope of this disclosure. The materials, methods, and examples provided here are representative of particular embodiments, are exemplary, and are not intended as limitations on the scope of the disclosure.

The scope of the disclosure has been described broadly and generically herein. Each of the narrower species and subgeneric groupings falling within the generic disclosure also form part of the disclosure. This includes the generic description with a proviso or negative limitation removing any subject matter from the genus, regardless of whether or not the excised material is specifically recited herein.

In addition, where features or aspects of the disclosure are described in terms of Markush groups, those skilled in the art will recognize that embodiments of the disclosure may also thereby be described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### CLAUSES

Clause 1. A targeted exon inclusion molecule comprising an optimized effector domain of an RNA-binding protein and an RNA-targeting moiety.

Clause 2. The molecule of clause 1, wherein the RNA-binding protein is selected from the group consisting of: SRSF8, RNPS1, SRSF10, SRSF4, SRSF5, SREK1, LUC7L2, SRSF6, SNIP1, U2AF2, GTF2F1, RBM25, STAU2, MAZ, CLK3, THRAP3, FIL1L1, MBNL1, SNRNP70, DDX23, XPO1, UBAP2L, SRSF12, RMBX2, SRSF11, PUG60, SNW1, METTL16, SF1, STAU1, CNOT3, EIF4B, SNRPN, SNRPB, SNRPA, RBM5, SNRNP40, RSRC1, TIAL1, FUBP1, SNURF, SRSF7, TRNAU1AP, CCNL1, SNRPE, RBFOX1, RBFOX2, KIAA1967, SNRPG, RTCA, CLK2, PRKRA, SCAF8, SF3A2, PCBP1, SF3B4, RBM38, RY1, and CELF3.

Clause 3. The molecule of clause 1 or 2, wherein the RNA-targeting moiety is selected from the group consisting of Cas13, Cas13 proteins with modifications to reduce immunogenicity, Pumilio (PUF) RNA binding proteins, antisense oligonucleotides (ASOs), or small molecule compounds.

Clause 4. A polynucleotide encoding the molecule of clause 1.

Clause 5. A vector or isolated host cell comprising the polynucleotide of clause 4.

Clause 6. A composition comprising the molecule of any one of clauses 1-3 and a carrier.

Clause 7. A composition comprising the polynucleotide of clause 5 and a carrier.

Clause 8. A method for targeted exon inclusion, the method comprising contacting a cell comprising a messenger RNA (mRNA) target with the molecule of clause 1 or 2, under conditions to allow the targeted exon inclusion molecule to bind to the mRNA target and facilitate inclusion of a target exon during splicing of the mRNA.

Clause 9. The method of clause 8, wherein the contacting of the cell occurs in vitro or in vivo.

Clause 10. The method of clause 8, wherein the cell is a mammalian cell.

Clause 11. The method of clause 10, wherein the mammalian cell is contacted in vitro and is a muscle cell.

Clause 12. The method of clause 11, wherein the muscle cell is from a subject with muscular dystrophy.

Clause 13. The method of clause 12, wherein the mRNA bound by the targeted exon inclusion molecule is dystrophin or a functional fragment thereof.

Clause 14. The method of clause 8, further comprising measuring the expression of the polynucleotide comprising the target exon.

Clause 15. The method of clause 12, further comprising measuring the levels of dystrophin protein produced from mRNA that include the target exon.

Clause 16. The method of clause 8, wherein the cell has been isolated from a subject suffering from a disease or disorder selected from muscular dystrophy, spinal muscular atrophy (SMA), Alzheimer's disease, familial dysautonomia, early-onset Parkinson's disease, X-linked parkinsonism with spasticity, cystic fibrosis, or CDKL5-deficiency disorder.

Clause 17. The method of clause 8, wherein a target sequence that is recognized by the RNA-targeting moiety of the targeted exon inclusion molecule is at most 30 nucleotides upstream or downstream of the target exon.

Clause 18. A method of generating targeted exon inclusion molecules, the method comprising: (a) generating a plurality of first fusion proteins, wherein each first fusion protein comprises an RNA-binding protein or fragment thereof and a reporter binding domain; (b) transfecting each of the first fusion proteins with a reporter construct, wherein the reporter construct comprises an mRNA sequence encoding a first reporter gene, a target exon comprising an in-frame stop codon, a reporter binding domain recognition sequence, and a second reporter gene, wherein the target exon is between the first reporter gene and the second reporter gene and wherein the reporter binding domain recognition sequence is either 30 nucleotides upstream or downstream of the target exon; (c) measuring the relative ratios of expression of the second reporter gene to expression of the first reporter gene for each transfection; (d) selecting the first fusion proteins that effectively direct targeted exon inclusion during splicing of the reporter construct, wherein effective direction of targeted exon inclusion is determined by a lower ratio of expression of the second reporter gene to expression of the first reporter gene; and (e) subcloning each RNA-binding protein or fragment thereof of the selected first fusion proteins with a tiling approach across the whole length of the RNA-binding protein or fragment thereof, or fusing each RNA-binding protein or fragment thereof to an RNA-targeting moiety to generate targeted exon inclusion molecules.

Clause 19. The method of clause 18, wherein upon subcloning of each RNA-binding protein or fragment thereof, the method further comprises: (f) generating a plurality of second fusion proteins, wherein each second fusion protein comprises a subcloned portion of the RNA-binding proteins or fragments thereof and the reporter binding domain; (g) transfecting each of the second fusion proteins with the reporter construct; (h) measuring the relative ratios of expression of the second reporter gene to the first reporter gene for each transfusion; (i) selecting the second fusion proteins that effectively direct targeted exon inclusion during splicing of the reporter construct; and (j) fusing each subcloned portion of the RNA-binding proteins or fragments thereof to an RNA-targeting moiety to generate additional targeted exon inclusion molecules.

### REFERENCES

ADDIN ZOTERO_BIBL {“uncited”: [ ], “omitted”: [ ], “custom”: [ ]} CSL_BIBLIOGRAPHY 1.
Gerstberger, S., Hafner, M. & Tuschl, T. A census of human RNA-binding proteins. Nat. Rev. Genet. 15, 829-845 (2014).
2. Queiroz, R. M. L. et al. Comprehensive identification of RNA-protein interactions in any organism using orthogonal organic phase separation (OOPS). Nat. Biotechnol. 37, 169-178 (2019).
3. Jiang, W. & Chen, L. Alternative splicing: Human disease and quantitative analysis from high-throughput sequencing. Comput. Struct. Biotechnol. J. 19, 183-195 (2021).
4. Wheeler, E. C. et al. Integrative RNA-omics Discovers GNAS Alternative Splicing as a Phenotypic Driver of Splicing Factor-Mutant Neoplasms. Cancer Discov. 12, 836-855 (2022).
5. Bradley, R. K. & Anczuków, O. RNA splicing dysregulation and the hallmarks of cancer. Nat. Rev. Cancer (2023) doi: 10.1038/s41568-022-00541-7.
6. Scotti, M. M. & Swanson, M. S. RNA mis-splicing in disease. Nat. Rev. Genet. 17, 19-32 (2016).
7. Rogalska, M. E., Vivori, C. & Valcárcel, J. Regulation of pre-mRNA splicing: roles in physiology and disease, and therapeutic prospects. Nat. Rev. Genet. (2022) doi: 10.1038/s41576-022-00556-8.
8. Zheng, S., Damoiseaux, R., Chen, L. & Black, D. L. A broadly applicable high-throughput screening strategy identifies new regulators of Dlg4 (Psd-95) alternative splicing. Genome Res. 23, 998-1007 (2013).
9. Moore, M. J., Wang, Q., Kennedy, C. J. & Silver, P. A. An Alternative Splicing Network Links Cell-Cycle Control to Apoptosis. Cell 142, 625-636 (2010).
10. Tejedor, J. R., Papasaikas, P. & Valcarcel, J. Genome-Wide Identification of Fas/CD95 Alternative Splicing Regulators Reveals Links with Iron Homeostasis. Mol. Cell 57, 23-38 (2015).
11. Sun, S., Zhang, Z., Fregoso, O. & Krainer, A. R. Mechanisms of activation and repression by the alternative splicing factors RBFOX1/2. RNA N. Y. N 18, 274-283 (2012).
12. Yeo, G. W. et al. An RNA code for the FOX2 splicing regulator revealed by mapping RNA-protein interactions in stem cells. Nat. Struct. Mol. Biol. 16, 130-137 (2009).
13. Lovci, M. T. et al. Rbfox proteins regulate alternative mRNA splicing through evolutionarily conserved RNA bridges. Nat. Struct. Mol. Biol. 20, 1434-1442 (2013).
14. Barash, Y. et al. Deciphering the splicing code. Nature 465, 53-59 (2010).
15. Tycko, J. et al. High-Throughput Discovery and Characterization of Human Transcriptional Effectors. Cell 183, 2020-2035.e16 (2020).
16. Luo, E.-C. et al. Large-scale tethered function assays identify factors that regulate mRNA stability and translation. Nat. Struct. Mol. Biol. 27, 989-1000 (2020).
17. Bos, T. J., Nussbacher, J. K., Aigner, S. & Yeo, G. W. Tethered Function Assays as Tools to Elucidate the Molecular Roles of RNA-Binding Proteins. in RNA Processing (ed. Yeo, G. W.) vol. 907 61-88 (Springer International Publishing, 2016).
18. Wang, Y., Cheong, C.-G., Tanaka Hall, T. M. & Wang, Z. Engineering splicing factors with designed specificities. Nat. Methods 6, 825-830 (2009).
19. Du, M., Jillette, N., Zhu, J. J., Li, S. & Cheng, A. W. CRISPR artificial splicing factors. Nat. Commun. 11, 2973 (2020).
20. Leclair, N. K. et al. Poison Exon Splicing Regulates a Coordinated Network of SR Protein Expression during Differentiation and Tumorigenesis. Mol. Cell 80, 648-665.e9 (2020).
21. Liu, F. & Gong, C.-X. Tau exon 10 alternative splicing and tauopathies. Mol. Neurodegener. 3, 8 (2008).
22. Popp, M. W. & Maquat, L. E. Leveraging Rules of Nonsense-Mediated mRNA Decay for Genome Engineering and Personalized Medicine. Cell 165, 1319-1322 (2016).
23. Chamieh, H., Ballut, L., Bonneau, F. & Le Hir, H. NMD factors UPF2 and UPF3 bridge UPF1 to the exon junction complex and stimulate its RNA helicase activity. Nat. Struct. Mol. Biol. 15, 85-93 (2008).
24. Boehm, V. et al. SMG5-SMG7 authorize nonsense-mediated mRNA decay by enabling SMG6 endonucleolytic activity. Nat. Commun. 12, 3965 (2021).
25. Binder, J. X. et al. COMPARTMENTS: unification and visualization of protein subcellular localization evidence. Database 2014, bau012-bau012 (2014).
26. Bondy-Chorney, E. et al. Staufen1 Regulates Multiple Alternative Splicing Events either Positively or Negatively in DM1 Indicating Its Role as a Disease Modifier. PLOS Genet. 12, e1005827 (2016).
27. Bondy-Chorney, E., Crawford Parks, T. E., Ravel-Chapuis, A., Jasmin, B. J. & Côté, J. Staufen1s role as a splicing factor and a disease modifier in Myotonic Dystrophy Type I. Rare Dis. 4, e1225644 (2016).
28. Van Nostrand, E. L. et al. A large-scale binding and functional map of human RNA-binding proteins. Nature 583, 711-719 (2020).
29. Ambrozková, M. et al. The Fission Yeast Ortholog of the Coregulator SKIP Interacts with the Small Subunit of U2AF. Biochem. Biophys. Res. Commun. 284, 1148-1154 (2001).
30. Selenko, P. et al. Structural Basis for the Molecular Recognition between Human Splicing Factors U2AF65 and SF1/mBBP. Mol. Cell 11, 965-976 (2003).
31. Matera, A. G. & Wang, Z. A day in the life of the spliceosome. Nat. Rev. Mol. Cell Biol. 15, 108-121 (2014).
32. Cvitkovic, I. & Jurica, M. S. Spliceosome Database: a tool for tracking components of the spliceosome. Nucleic Acids Res. 41, D132-D141 (2013).
33. Chen, Y.-I. G. et al. Proteomic analysis of in vivo-assembled pre-mRNA splicing complexes expands the catalog of participating factors. Nucleic Acids Res. 35, 3928-3944 (2007).
34. Ajuh, P. Functional analysis of the human CDC5L complex and identification of its components by mass spectrometry. EMBO J. 19, 6569-6581 (2000).
35. McCracken, S. et al. Proteomic Analysis of SRm160-containing Complexes Reveals a Conserved Association with Cohesin. J. Biol. Chem. 280, 42227-42236 (2005).
36. Sharma, S., Kohlstaedt, L. A., Damianov, A., Rio, D. C. & Black, D. L. Polypyrimidine tract binding protein controls the transition from exon definition to an intron defined spliceosome. Nat. Struct. Mol. Biol. 15, 183-191 (2008).
37. Rappsilber, J., Ryder, U., Lamond, A. I. & Mann, M. Large-Scale Proteomic Analysis of the Human Spliceosome. Genome Res. 12, 1231-1245 (2002).
38. Azizian, N. G. & Li, Y. XPO1-dependent nuclear export as a target for cancer therapy. J. Hematol. Oncol. J Hematol Oncol 13, 61 (2020).
39. Heraud-Farlow, J. E. et al. Staufen2 Regulates Neuronal Target RNAs. Cell Rep. 5, 1511-1518 (2013).
40. Almasi, S. & Jasmin, B. J. The multifunctional RNA-binding protein Staufen1: an emerging regulator of oncogenesis through its various roles in key cellular events. Cell. Mol. Life Sci. 78, 7145-7160 (2021).
41. Yuryev, A. et al. The C-terminal domain of the largest subunit of RNA polymerase II interacts with a novel set of serine/arginine-rich proteins. Proc. Natl. Acad. Sci. 93, 6975-6980 (1996).
42. Tanaka, N. & Shuman, S. Structure-activity relationships in human RNA 3′-phosphate cyclase. RNA 15, 1865-1874 (2009).
43. Hu, X. et al. Knockdown of Trnaulap inhibits the proliferation and migration of NIH3T3, JEG-3 and Bewo cells via the PI3K/Akt signaling pathway. Biochem. Biophys. Res. Commun. 503, 521-527 (2018).
44. Van Nostrand, E. L. et al. Robust transcriptome-wide discovery of RNA-binding protein binding sites with enhanced CLIP (eCLIP). Nat. Methods 13, 508-514 (2016).
45. Luo, Y. et al. New developments on the Encyclopedia of DNA Elements (ENCODE) data portal. Nucleic Acids Res. 48, D882-D889 (2020).
46. Boyle, E. A. et al. Skipper analysis of eCLIP datasets enables sensitive detection of constrained translation factor binding sites. Cell Genomics 100317 (2023) doi: 10.1016/j.xgen.2023.100317.
47. Fairbrother, W. G., Yeh, R.-F., Sharp, P. A. & Burge, C. B. Predictive Identification of Exonic Splicing Enhancers in Human Genes. Science 297, 1007-1013 (2002).
48. Xiao, X. et al. Splice site strength-dependent activity and genetic buffering by poly-G runs. Nat. Struct. Mol. Biol. 16, 1094-1100 (2009).
49. Georgakopoulos-Soares, I. et al. Alternative splicing modulation by G-quadruplexes. Nat. Commun. 13, 2404 (2022).
50. Warf, M. B., Diegel, J. V., Von Hippel, P. H. & Berglund, J. A. The protein factors MBNL1 and U2AF65 bind alternative RNA structures to regulate splicing. Proc. Natl. Acad. Sci. 106, 9203-9208 (2009).
51. Street, L. et al. Large-scale map of RNA binding protein interactomes across the mRNA life-cycle. http://biorxiv.org/lookup/doi/10.1101/2023.06.08.544225 (2023) doi: 10.1101/2023.06.08.544225.
52. Han, J. et al. Multilayered control of splicing regulatory networks by DAP3 leads to widespread alternative splicing changes in cancer. Nat. Commun. 13, 1793 (2022).
53. Chen, X. et al. Context-defined cancer co-dependency mapping identifies a functional interplay between PRC2 and MLL-MENI complex in lymphoma. Nat. Commun. 14, 4259 (2023).
54. Uhlén, M. et al. Tissue-based map of the human proteome. Science 347, 1260419 (2015).
55. Rauch, S. et al. Programmable RNA-Guided RNA Effector Proteins Built from Human Parts. Cell 178, 122-134.e12 (2019).
56. Zhou, Y. et al. Metascape provides a biologist-oriented resource for the analysis of systems-level datasets. Nat. Commun. 10, 1523 (2019).
57. Heinz, S. et al. Simple Combinations of Lineage-Determining Transcription Factors Prime cis-Regulatory Elements Required for Macrophage and B Cell Identities. Mol. Cell 38, 576-589 (2010).
58. Rual, J.-F. et al. Human ORFeome Version 1.1: A Platform for Reverse Proteomics. Genome Res. 14, 2128-2135 (2004).
59. Blum, M. et al. The InterPro protein families and domains database: 20 years on. Nucleic Acids Res. 49, D344-D354 (2021).
60. Team, T. P. D. pandas-dev/pandas: Pandas. (2023) doi: 10.5281/ZENODO.3509134.
61. Virtanen, P. et al. SciPy 1.0: fundamental algorithms for scientific computing in Python. Nat. Methods 17, 261-272 (2020).
62. Schneider, C. A., Rasband, W. S. & Eliceiri, K. W. NIH Image to ImageJ: 25 years of image analysis. Nat. Methods 9, 671-675 (2012).
63. Steffen Durinck <Biomartdev@Gmail. Com>, W. H. biomaRt. (2017) doi: 10.18129/B9.BIOC.BIOMART.
64. Frankish, A. et al. GENCODE reference annotation for the human and mouse genomes. Nucleic Acids Res. 47, D766-D773 (2019).
65. Love, M. I., Huber, W. & Anders, S. Moderated estimation of fold change and dispersion for RNA-seq data with DESeq2. Genome Biol. 15, 550 (2014).
66. Shen, S. et al. rMATS: Robust and flexible detection of differential alternative splicing from replicate RNA-Seq data. Proc. Natl. Acad. Sci. 111, (2014).
67. Bruderer, R. et al. Extending the Limits of Quantitative Proteome Profiling with Data-Independent Acquisition and Application to Acetaminophen-Treated Three-Dimensional Liver Microtissues. Mol. Cell. Proteomics 14, 1400-1410 (2015).
68. Wessels, H.-H. et al. Massively parallel Cas13 screens reveal principles for guide RNA design. Nat. Biotechnol. 38, 722-727 (2020).
69. Guo, X. et al. Transcriptome-wide Cas13 guide RNA design for model organisms and viral RNA pathogens. Cell Genomics 1, 100001 (2021).
70. Schmok, J. C. et al. Systematic identification of RNA-binding proteins and tethered domains that activate exon splicing inclusion. Gene Expression Omnibus. https://www.ncbi.nlm.nih.gov/geo/query/acc.cgi?acc-GSE232599 (2023).

What is claimed is:

1. A targeted exon inclusion molecule comprising an optimized effector domain of an RNA-binding protein and an RNA-targeting moiety.

2. The molecule of claim 1, wherein the RNA-binding protein is selected from the group consisting of: SRSF8, RNPS1, SRSF10, SRSF4, SRSF5, SREK1, LUC7L2, SRSF6, SNIP1, U2AF2, GTF2F1, RBM25, STAU2, MAZ, CLK3, THRAP3, FILIL1, MBNL1, SNRNP70, DDX23, XPO1, UBAP2L, SRSF12, RMBX2, SRSF11, PUG60, SNW1, METTL16, SF1, STAU1, CNOT3, EIF4B, SNRPN, SNRPB, SNRPA, RBM5, SNRNP40, RSRC1, TIAL1, FUBP1, SNURF, SRSF7, TRNAU1AP, CCNL1, SNRPE, RBFOX1, RBFOX2, KIAA1967, SNRPG, RTCA, CLK2, PRKRA, SCAF8, SF3A2, PCBP1, SF3B4, RBM38, RY1, and CELF3.

3. The molecule of claim 1, wherein the RNA-targeting moiety is selected from the group consisting of Cas13, Cas13 proteins with modifications to reduce immunogenicity, Pumilio (PUF) RNA binding proteins, antisense oligonucleotides (ASOs), or small molecule compounds.

4. A polynucleotide encoding the molecule of claim 1.

5. A vector or isolated host cell comprising the polynucleotide of claim 4.

6. A composition comprising the molecule of claim 1 and a carrier.

7. A composition comprising the polynucleotide of claim 5 and a carrier.

8. A method for targeted exon inclusion, the method comprising contacting a cell comprising a messenger RNA (mRNA) target with the molecule of claim 1, under conditions to allow the targeted exon inclusion molecule to bind to the mRNA target and facilitate inclusion of a target exon during splicing of the mRNA.

9. The method of claim 8, wherein the contacting of the cell occurs in vitro or in vivo.

10. The method of claim 8, wherein the cell is a mammalian cell.

11. The method of claim 10, wherein the mammalian cell is contacted in vitro and is a muscle cell.

12. The method of claim 11, wherein the muscle cell is from a subject with muscular dystrophy.

13. The method of claim 12, wherein the mRNA bound by the targeted exon inclusion molecule is dystrophin or a functional fragment thereof.

14. The method of claim 8, further comprising measuring the expression of the polynucleotide comprising the target exon.

15. The method of claim 12, further comprising measuring the levels of dystrophin protein produced from mRNA that include the target exon.

16. The method of claim 8, wherein the cell has been isolated from a subject suffering from a disease or disorder selected from muscular dystrophy, spinal muscular atrophy (SMA), Alzheimer's disease, familial dysautonomia, early-onset Parkinson's disease, X-linked parkinsonism with spasticity, cystic fibrosis, or CDKL5-deficiency disorder.

17. The method of claim 8, wherein a target sequence that is recognized by the RNA-targeting moiety of the targeted exon inclusion molecule is at most 30 nucleotides upstream or downstream of the target exon.

18. A method of generating targeted exon inclusion molecules, the method comprising:
(a) generating a plurality of first fusion proteins, wherein each first fusion protein comprises an RNA-binding protein or fragment thereof and a reporter binding domain;
(b) transfecting each of the first fusion proteins with a reporter construct, wherein the reporter construct comprises an mRNA sequence encoding a first reporter gene, a target exon comprising an in-frame stop codon, a reporter binding domain recognition sequence, and a second reporter gene, wherein the target exon is between the first reporter gene and the second reporter gene and wherein the reporter binding domain recognition sequence is either 30 nucleotides upstream or downstream of the target exon;
(c) measuring the relative ratios of expression of the second reporter gene to expression of the first reporter gene for each transfection;
(d) selecting the first fusion proteins that effectively direct targeted exon inclusion during splicing of the reporter construct, wherein effective direction of targeted exon inclusion is determined by a lower ratio of expression of the second reporter gene to expression of the first reporter gene; and
(e) subcloning each RNA-binding protein or fragment thereof of the selected first fusion proteins with a tiling approach across the whole length of the RNA-binding protein or fragment thereof, or fusing each RNA-binding protein or fragment thereof to an RNA-targeting moiety to generate targeted exon inclusion molecules.

19. The method of claim 18, wherein upon subcloning of each RNA-binding protein or fragment thereof, the method further comprises:
(f) generating a plurality of second fusion proteins, wherein each second fusion protein comprises a subcloned portion of the RNA-binding proteins or fragments thereof and the reporter binding domain;
(g) transfecting each of the second fusion proteins with the reporter construct;
(h) measuring the relative ratios of expression of the second reporter gene to the first reporter gene for each transfusion;
(i) selecting the second fusion proteins that effectively direct targeted exon inclusion during splicing of the reporter construct; and
(j) fusing each subcloned portion of the RNA-binding proteins or fragments thereof to an RNA-targeting moiety to generate additional targeted exon inclusion molecules.
